(19) United States
(12) Patent Application Publication
(10) Pub. No.: US 2017/0056468 A1
(43) Pub. Date: Mar. 2, 2017

(54) TREATMENT OF RESISTANT LESIONS

(71) Applicant: CoDa Therapeutics, Inc., San Diego, CA (US)

(72) Inventors: David EISENBUD, San Diego, CA (US); Anthony PHILLIPS, San Diego, CA (US); Scott BANNAN, San Diego, CA (US); Grove MATSUOKA, San Diego, CA (US); DUFT Bradford, San Diego, CA (US)

(21) Appl. No.: 15/247,853

(22) Filed: Aug. 25, 2016

Related U.S. Application Data
(63) Continuation of application No. PCT/US2015/020786, filed on Mar. 16, 2015.
Application No. PCT/US2015/020786 is a continuation-in-part of application No. PCT/US2015/017595, filed on Feb. 25, 2015.
(60) Provisional application No. 61/953,604, filed on Mar. 14, 2014.
(60) Provisional application No. 61/953,608, filed on Mar. 14, 2014.
(60) Provisional application No. 61/944,566, filed on Feb. 25, 2014.

(51) Int. Cl. A61K 38/17; A61K 9/06; A61K 47/10; C12N 15/113

(52) U.S. Cl. CPC A61K 38/177; C12N 15/1138; A61K 9/06; A61K 47/10; C12N 2310/11; C12N 2320/30

(57) ABSTRACT

Connexin protein modulation methods and compositions are provided for the healing of resistant lesions, including lesions on subjects with multiple venous leg ulcers or multiple diabetic foot ulcers, and other responder subjects. Also provided are kits and articles of manufacture comprising a connexin protein modulating agent, for example, a connexin 43 modulating agent for use in the healing of resistant lesions.

### TECHNICAL FIELD

The inventions relate to the treatment of resistant skin lesions. The inventions are useful in various contexts, including to promote healing in subjects with multiple venous leg ulcers and multiple diabetic foot ulcers, for example.

### RELATED APPLICATIONS

This Application claims benefit of U.S. Provisional Application No. 61/953,604, filed on Mar. 14, 2014, and of US Provisional application Ser. No. 51/953,608, filed on Mar. 14, 2014, the contents of each of which are hereby expressly incorporated by reference as if set forth in their entirety.

### BACKGROUND

Normal wound healing moves through phases in a timely and uncomplicated fashion (hemostasis, inflammation, proliferation, epithelialization, and remodeling/maturation). Wounds that do not heal normally or at expected rates, typically those that have been present from more than one to three or six months, deviate from the expected sequence of repair. They are sometimes also referred to as ulcers, and include venous leg ulcers and diabetic foot ulcers.

Diabetic foot ulcers are a common and much feared complication of diabetes. Diabetic foot ulcers (DFUs) have major short- and long-term impacts on patients' quality of life, morbidity and mortality. Studies suggest that the lifetime risk of developing a foot ulcer in diabetic patients may be as high as 25%. Foot ulceration requires long and intensive treatment, and is associated with major healthcare costs. According to the Center for Disease Control, diabetes is the leading cause of nontraumatic lower-limb amputations in the United States. Mortality is high and healed ulcers often recur. Prompers, L. et al., Prediction of outcome in individuals with diabetic foot ulcers: focus on the differences between individuals with and without peripheral arterial disease. The EURODIALE Study. Diabetologia. 2008 May; 51(5): 747-755. Despite good management, DFU healing rates in large multicentre trials were reported to be 24% at 12 weeks and 31% at 20 weeks. Margolis D J, et al. Healing of diabetic neuropathic foot ulcers receiving standard treatment: a meta-analysis. Diab Care 1999; 22: 692-95. Even advanced modalities such as skin substitutes or growth factors are said to have demonstrated, at best, a 56% healing rate within 12 weeks. Shishir Shah, D O, Clinical and Economic Benefits of Healing Diabetic Foot Ulcers With a Rigid Total Contact Cast. Wounds. 2012; 24(6):152-159. One study that examined a total of 1,000 consecutive DFU patients between December 1997 and April 2004 reported that 40% had multiple diabetic foot ulcers (multiple or mDFUs). Wounds on the mDFU patients reportedly had a significantly lower probability of healing (p<0.00001), and multivariate analysis confirmed this parameter as an independent variable with a significant impact on healing. Beckert S, et al. A New Wound-Based Severity Score for Diabetic Foot Ulcers: A prospective analysis of 1,000 patients. Wounds. 2012; 24(6): 152-159.

Approximately 70%-80% of ulcers of the lower limbs are venous leg ulcers (VLUs) (Abbade, Venous ulcer: epidemiology, physiopathology, diagnosis and treatment, Internal J Dermatol. 2005; 44:449-456 (2005); O'Brien, et al., Prevalence and aetology of leg ulcers in Ireland, Ir J Med Sci. 2000; 169:110-112 (2000). In the United States, VLUs are commonly associated with substantial disability, impaired quality of life, and high economic costs. Heber, et al., A systematic review on the impact of leg ulceration on patients' quality of life, Health and Quality of Life Outcomes 5:44 (2007). The slow rate at which many VLU patients heal prolongs these problems. Compression therapy, which is applied to improve venous circulation, has remained the standard care for VLUs over several decades but is often insufficient to heal VLUs in a timely manner. One recent report shows that just 61.5% of patients healed at one year in clinical trials (Rippon, M., et al, The economic impact of Chronic Wounds, Wounds UK, 2007, 3, No 2). Nevertheless, compression bandaging remains the standard of care (SOC) due to a lack of effective alternatives. VLU-related treatment costs are directly related to time to achieve complete wound closure.

As with DFU patients, some VLU patients have more than one wound at the same time (multiple or mVLUs). Like mDFUs, multiple VLUs are considered more difficult to heal than single VLUs (sVLU), and increasing VLU number has been associated with worse outcome. Margolis et al., Venous leg ulcer: incidence and prevalence in the elderly, Wound Rep Reg 2004; 12:163-168. The incidence of mVLU appears to be increasing. Analysis of an Intellicure Chronic Wound Dataset shows that 54% of patients treated at wound care centers using the U.S. Wound Registry medical database from 2007 to 2012 had multiple VLUs vs. 40% as reported by Margolis et al. (2004) from a 1998-2000 dataset. Thus, reports indicate that the incidence of mVLU has grown significantly, by almost 15%, in just over a decade.

Gap junctions are a unique type of intercellular communication conduit found in most animal cell types. They form channels that interconnect the cytoplasms of adjacent cells and permit the direct, cell-to-cell exchange of ions, secondary messengers, water, electrical impulses and low-molecular-weight metabolites and nutrients, thereby coordinating diverse metabolic and electrical functions of cell communities. Gap junctions cross the extracellular space between cells by the docking of two hemichannels (connexons). One connexon is contributed by each adjacent cell. Each connexon is an oligomer of six connexin monomers surrounding a central pore.

Human connexins are a polygenic family of 21 transmembrane proteins, and each is believed to provide permeability and regulatory properties to the channels they form. The most prevalent human connexin is connexin 43 (Coutinho et al., Dynamic changes in connexin expression correlate with key events in the wound healing process, Cell Biol Int. 27:525-554 (2003). Connexin 43 is the predominant connexin in human epidermis (Salomon et al., Topography of mammalian connexins in human skin. J Invest Dermatol, 1994 103, 240-247) and, after acute cutaneous injury, its expression pattern changes dynamically at the edges of acute wounds during the wound-healing process (Coutinho et al., 2003). Levels of connexin 43 initially decrease at the wound edge (Goliger & Paul, Wounding alters epidermal connexin expression and gap junction mediated intercellular communication, Mol Biol Cell. 6:1491-1501 (1995); Saitoh et al. Changes in the expression of gap junction proteins (connexins) in hamster tongue epithelium during would healing and carcinogenesis, Carcinogenesis 18:1319-1328 (1997)) but increase in more distant regions where cells are proliferating (Coutinho et al., 2003; Goliger & Paul, 1995). Down-regulation of connexin 43 has been shown to occur during normal healing of acute wounds. Connexin 43 is down-regulated in acute wound edge keratinocytes and dermal fibroblasts as they become migratory (Coutinho 2003; Goliger & Paul 1995).

Connexin 26 is found in cells throughout the body, including the inner ear and the skin. Some studies indicate that channels made with connexin 26 help to maintain the correct level of potassium ions. Other research suggests that connexin 26 is required for the maturation of certain cells in the cochlea. Connexin 26 is also reported to play a role in the growth, maturation, and stability of the outermost layer of skin (the epidermis). Connexin 30 is also found in several different tissues throughout the body, including the brain, skin, and inner ear. Some studies indicate that gap junctions made with connexin 30 also help to maintain the correct level of potassium ions. Connexin 30 gap junctions are also said to play a role in the growth and maturation of the epidermis.

### DESCRIPTION OF THE DRAWINGS

FIG. 1A-FIG. 1B represent blood vessels in VLUs. Blood vessel staining (green) ihn a representative (FIG. 1A) intact arm skin biopsy and (FIG. 1B) VLU. Chronic wound tissue is characterized by an enhanced number of dermal blood vessels. Scale bars—100 and 500 μm respectively. Cx43=Red; Blood vessels/alpha smooth muscle actin=Green; Nuclei and autofluorescent extracellular matrix=Blue. VLU, venous leg ulcer; Cx, connexin.

FIG. 2A-FIG. 2Y represent Cx43, Cx26 and Cx30 expression in VLUs. (FIG. 2A and FIG. 2C) Location of Cx43 quantification sites. (FIG. 2B) Representative VLU. (FIG. 2D-FIG. 2E) Mean of the individual and group Cx fold changes compared directly to the reference arm values. (FIG. 2F-FIG. 2Y) Cx43, 26 and 30 expression and associated summary graphs for the WE, 1 mm from the WE and at the FE. Scale bar—10× Motages 1000 μm, 40× Images 100 μm. Cx43, 26 and 30=Green; Nuclei=Blue. **p<0.01; ***p<0.001. Error bars—Mean+/−SEM (Epidermis—n=19 except FE=14; Dermis—n=17). VLU, venous leg ulcer; Cx, connexin; WE, wound edge; FE, far edge.

FIG. 3A-FIG. 3Y represent Cx43, Cx26 and Cx30 expression in DFUs. (FIG. 3A and FIG. 3C) Location of Cx43 quantification sites. (FIG. 3B) Representative DFU. (FIG. 3D-FIG. 3E) Mean of the individual and group Cx fold changes compared directly to the reference arm values. (FIG. 3F-FIG. 3Y) Cx43, 26 and 30 expression and associated summary graphs for the WE, 1 mm from the WE and at the FE. Scale bar—10× Montages 1000 μm 40× Images 100 μm. Cx43, 26 and 30=Green; Nuclei=Blue. *p<0.05; **p<0.01; ***p<0.001. Error bars—Mean+/−SEM (Epidermis—n=11 except FE=8; Dermis—n=6). DFU, diabetic foot ulcer; Cx, connexin; WE, wound edge; FE, far edge.

FIG. 4A-FIG. 4Y represent Cx43, Cx26 and Cx30 expression in PRUs. (FIG. 4A and FIG. 4C) Location of Cx43 quantification sites. (FIG. 4B) Representative PRU. (FIG. 4D-FIG. 4E) Mean of the individual and group Cx fold changes compared directly to the reference arm values. (FIG. 4F-FIG. 4Y) Cx43, 26 and 30 expression and associated summary graphs for the WE, 1 mm from the WE and at the FE. Scale bar—10× Montages 1000 μm; 40× Images 100 μm. Cx43, 26 and 30=Green; Nuclei=Blue. *p<0.05; **p<0.01; ***p<0.001. Error bars—Mean+/−SEM (Epidermis and dermis—n=6 except FE=5). PRU, pressure ulcer; Cx, connexin; WE, wound edge; FE, far edge.

FIG. 5 shows where assessments were taken across a 4 mm biopsy using an Olympus FV-1000 inverted confocal microscope to take 40× images of arm skin and wound.

FIG. 6 shows Cx43 in dermis normalised to patient baseline expression.

### BRIEF SUMMARY OF THE INVENTION

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

In one aspect this invention relates to pharmaceutical formulations and methods for treating resistant lesions with a connexin protein modulating agent.

In another aspect the invention relates to pharmaceutical formulations and methods for treating lesions on subjects likely to be responsive to treatment with a connexin protein modulating agent, based on indicators including those described herein. Such factors include, for example, the presence of multiple venous leg ulcers (mVLUs) on a subject, and the presence of multiple diabetic foot ulcers (mDFUs). Other factors include degree of local or systemic inflammation in or on a subject, including lesion or wound inflammation, as described herein. Another factor which may be considered in combination with other factors indicative of resistant lesions includes the amount of healing during a pretreatment or run-in period with a standard-of-care treatment, and the amount healing during a pretreatment or run-in period with standard-of-care treatment together with a hydrogel or other product applied to the wound to maintain a moist wound environment, all as measured by, for example, percent wound surface area reduction and/or linear wound advance. Yet other factors include the size of the lesion or the duration of the lesion or both.

In one aspect this invention relates to compositions and methods for treating an ulcer or lesion on a mVLU or mDFU subject by administering a connexin protein modulating agent in amounts effective to promote healing. This invention also relates to methods of determining whether subjects are responder subjects likely to respond to treatment by a connexin protein modulating agent, based on indicators including the presence of mVLUs or mDFUs, for example. It has been found that patients with multiple lesions, or multiple resistant lesions, who generally have a poorer prognosis for healing using standard treatments, respond surprisingly well to treatment by, for example, connexin 43 modulating agents.

In one aspect, this invention relates to the treatment of resistant lesions and responder subjects including subjects who have mVLUs or mDFUs, which are more resistant to healing, by administering a therapeutically effective amount of a composition comprising, for example, a connexin 43 modulating agent. The compositions and methods relate in part to the surprising discovery that patients with resistant lesions, including, for example, patients with mVLUs and mDFUs, respond particularly well, and far better than standard-of-care or vehicle plus standard-of-care, to treatment with a connexin protein modulating agent in a dose dependent manner, in contrast to other subjects, for example, those having a single VLU or single DFU, for whom treatment with a connexin protein modulating agent shows less effect over treatment with vehicle plus standard-of-care or standard-of-care alone. Thus, it has been suprisingly discovered, in the case of VLU, for example, that although subjects having multiple lesions generally have a low likelihood of response to treatment with compression bandaging, they are very responsive to treatment with a connexin protein modulating agent, for example, a connexin 43 modulating agent.

In some embodiments the connexin protein modulating agent is a connexin protein antisense oligonucleotide. In one embodiment the connexin protein modulating agent is a connexin protein antisense oligodeoxynucleotide, whether chemically modified or unmodified. In some aspects the therapeutically effective amount of the connexin protein modulating agent is any amount effective to promote healing of a resistant lesion in or on a subject. Connexin 43, connexin 26 and connexin 30 protein modulating agents are preferred. Connexin 43 protein modulating agents are particularly preferred.

Examples of effective doses that may be used for the treatment of resistant lesions are described and claimed herein. In some aspects, the therapeutically effective amount effective to promote healing of a resistant lesion in or on a subject is administered, for example, by applying, coating or filling the lesion with a connexin protein modulating agent present at a concentration of about 0.1 mg/mL to about 100 mg/mL, or more. In other embodiments, the connexin protein modulating agent is present at a concentration ranging from about 0.5 to about 50 mg/mL. In other embodiments, the connexin protein modulating agent is present at a concentration ranging from about 0.3 to about 30 mg/mL. In other embodiments, the connexin protein modulating agent is present at a concentration ranging from about 0.1 or 1.0 to about 10 mg/mL. In other embodiments, the connexin protein modulating agent is present at a concentration ranging from about 0.1 or 1.0 to about 0.3 or 3.0 mg/mL. In other embodiments, the connexin protein modulating agent is present at a concentration of about 3.0 mg/mL. In any of these aspects the connexin protein modulating agent may be a connexin protein antisense oligonucleotide. When the connexin protein modulating agent is a modified connexin protein antisense oligonucleotide, e.g., a backbone-modified oligonucleotide, or chemically modified oligonucleotide for increased half-life, the above-noted dose concentrations may be the same, or may be decreased or increased as appropriate based on potency and specificity, for example. In any of these aspects, the carrier (vehicle) may be a pharmaceutically acceptable carrier. Such carries include poloxamer gel, for example, poloxamer 407, present in an amount ranging from about 15% to 25%, or 20% to 30%, for example.

In some aspects, this invention also relates to methods of determining whether subjects are those likely to respond to treatment by a connexin protein modulating agent, based on indicators described herein. Such factors include, for example, as noted, the presence of multiple ulcers, inflammation, the amount of healing measured during a pretreatment or run-in period with standard-of-care treatment and/or a product to maintain moisture at the lesion, as well as the size and/or duration of the lesion. In one aspect, in the case of VLUs, the indicator is the presence of mVLUs, as noted.

According to the invention, other indicators that have also surprisingly been discovered to increase the likelihood of complete healing in response to treatment of a VLU on a patient with more than one lesion using a connexin protein modulating agent include, for example, age of the subject and body mass index (BMI). For example, in some embodiments, the indicator can be age over 50. In one embodiment the age indicator can include, for example, ages over 50-52 years. A body mass index (BMI) of less than 40 or 42, for example, is another indicator discovered to affect likelihood of response to treatment by a connexin protein modulating agent, for example, a connexin 43 modulating agent. In one embodiment the BMI indicator is less than 40. In some embodiments, subjects over 50 having mVLUs and a BMI of less than 42 exhibit a dose dependent significant response to a connexin protein modulating agent such as a connexin protein antisense oligodeoxynucleotide, and are up to 5 times more likely to heal, or greater, than mVLU subjects over 50 who are treated with standard of care.

In one aspect the compositions of this invention comprise one or more anti-connexin protein, for example, anti-connexin 43, polynucleotides that modulate connexin activity. In some aspects the connexin modulating agent may be antisense oligonucleotides. Anti-connexin oligonucleotides can inhibit connexin activity by decreasing its expression. In one aspect the active ingredient includes a connexin protein, for example, anti-connexin 43, modulating agent. In other aspects the connexin protein modulating agents may be anti-connexin peptides, peptidomimetics (for example, anti-connexin 43 peptides or peptidomimetics), gap junction closing compounds, hemichannel closing compounds, and connexin carboxy-terminal polypeptides for use in treating subjects with resistant lesions.

The connexin modulating agents of this invention may be used alone or in combination. In some embodiments, treatment with a connexin protein modulating agent is administered in conjunction with standard-of-care, for example, compression bandaging and/or off-loading.

The invention includes a package or kit comprising a pharmaceutical composition comprising a pharmaceutically acceptable carrier and a pharmaceutically acceptable anti-connexin modulating agent, together with a label and/or instructions for administering the composition to subjects with resistant lesions, for example where the subject has mVLUs or mDFUs, and the agent is administered in amounts effective to promote healing of the lesions in a subject, alone or together with standard-of-care, for example, compression bandaging and/or off-loading. In one embodiment, the invention includes a package or kit comprising a pharmaceutical composition including a pharmaceutically acceptable carrier and a pharmaceutically acceptable anti-connexin protein modulating agent, such as an anti-connexin protein oligonucleotide, optionally with a label and/or instructions for administering the composition to responder subjects with mVLUs and/or mDFUs in amounts effective to promote mVLU and/or mDFU healing in a subject, alone or under compression bandaging. Packages and kits include those with a connexin 43 protein modulating agent, a connexin 26 protein modulating agent and/or a connexin 30 protein modulating agent.

### DETAILED DESCRIPTION

In one embodiment this invention relates to methods for treating responder subjects and subjects with resistant lesions, i.e., one or more resistant lesions, and compositions useful in those methods. The compositions may include pharmaceutical formulations or dosage forms, suitable for administration in therapeutically effective amounts.

In one embodiment, the compositions and methods are based on the surprising discovery that certain subjects including subjects with resistant lesions, such as, for example, patients with mVLUs and mDFUs, respond particularly well, and far better than standard-of-care or vehicle plus standard-of-care, to treatment with a connexin protein modulating agent in a dose dependent manner, in contrast to other subjects, for example, those having a single VLU or a single DFU, for whom treatment with a connexin protein modulating agent shows less effect over treatment with vehicle plus standard-of-care or standard-of-care alone. Thus, it has been suprisingly found, in the case of VLU, for example, that although subjects having multiple lesions generally have a low likelihood of response to treatment with compression bandaging, they are very responsive to treatment with a connexin 43 modulating agent. As described further herein, it has been surprisingly discovered that lesions on subjects having resistant lesions, such as mVLUs, are more than three times more likely to completely heal following treatment with a connexin 43 modulating agent than a lesion on an mVLU subject treated with standard of care or vehicle, even though wounds on patients having mVLUs usually have a low likelihood of complete healing as described herein. In some embodiments, the connexin modulating agent is a connexin 43, connexin 30 or connexin 26 antisense oligonucleotide. In one embodiment the connexin 43 modulating agent is a modified connexin 43 antisense oligodeoxynucleotide.

Accordingly, in one embodiment, this invention relates to compositions and methods useful in treating subjects with resistant lesions, including, for example, mVLUs and mDFUs. Compositions and formulations useful in the invention include a connexin protein modulating agent. Particular formulations include connexin 43 modulating agents, connexin 26 modulating agents, and connexin 30 modulating agents.

In another embodiment, the invention relates to pharmaceutical formulations and methods for treating a wound on a subject having multiple venous leg ulcers (mVLUs), i.e., more than one venous leg ulcer at the same time, or other resistant lesions in responder subjects likely to respond to treatment by a connexin protein modulating agent, as described herein. In another embodiment, the invention relates to pharmaceutical formulations and methods for treating a wound on a subject having multiple diabetic foot ulcers (mDFUs), i.e., more than one diabetic foot ulcer at the same time, or other resistant lesions in diabetic responder subjects likely to respond to treatment by a connexin 43 modulating agent, as described herein. In either case, the one or more ulcers can be on the same leg, or on different legs. In either case, the subject may be over about 50 years of age, and/or have a BMI of less than about 40.

It has been found that patients with multiple resistant lesions such as mVLUs, who generally have a poorer prognosis for healing using standard treatments than those with single lesions such as sVLUs, have a surprising comparatively high likelihood of responding to treatment by connexin 43 modulating agents. Accordingly, in some embodiments, this invention relates to treating patients with mVLUs by administering an amount of a composition comprising a connexin 43 modulating agent in an amount effective to promote VLU healing in an mVLU subject. In some embodiments the connexin 43 modulating agent is a connexin 43 antisense oligonucleotide. The connexin 43 antisense oligonucleotide may be, in some embodiment, an unmodified connexin 43 antisense oligodeoxynucleotide.

Thus, in one embodiment, this invention relates to treating responder subjects who have resistant lesions such as mVLUs or mDFUs. This invention relates in one aspect to formulations and methods for treating resistant lesions in subjects who are likely to be responsive to treatment by a connexin 43 modulating agent, based on indicators including those described herein, which include the presence of mVLUs or mDFUs, or, in some instances, the presence of a biomarker indicative of a resistant lesion, as discussed above.

In one aspect, this invention relates to formulations and methods of treating responder subjects who have resistant lesions such as mVLUs by administering a therapeutically effective amount of a composition comprising a connexin 43 modulating agent to responder subjects.

According to this invention, indicators in addition to presence of mVLUs which have further suprisingly been found to increase the likelihood of complete healing in response to treatment for resistant lesions using a connexin 43 modulating agent include, for example, age of the subject and body mass index (BMI). For example, in some embodiments, the indicator can be age over 50. In one embodiment the age indicator can include, for example, age over 52. A body mass index (BMI) of less than 42 is another indicator found to affect susceptibility to treatment by a connexin 43 modulating agent. In one embodiment the BMI indicator is a BMI less than 40, preferably a BMI less than 35 or 30, and most preferably a BMI less than 25. In some embodiments, subjects aged over 50 (or 52) having mVLUs and a BMI of less than 40 (or 42) exhibit a dose dependent significant response to a connexin 43 modulating agent such as a connexin 43 antisense oligodeoxynucleotide.

Although the presence of mVLUs and sVLUs in older subjects have been associated with more difficulty in healing and a lower likelihood of complete healing, it has been surprisingly discovered that the difficult to heal subjects over 50 with mVLUs are more likely to respond to treatment with a connexin 43 modulating agent than mVLU subjects over 50 who are treated with vehicle and/or standard-of-care. For example, mVLU subjects over 50 show a dose dependent response following treatment with an anti-connexin 43 modulating agent, such as a connexin 43 antisense oligonucleotide, are up to 5 times or more likely to heal than mVLU subjects over 50, for example, those over about 52 years of age, who are treated with standard-of-care alone.

Although the presence of mVLUs and sVLUs in older subjects have been associated with more difficulty in healing and a lower likelihood of complete healing, it has been surprisingly discovered that the difficult to heal subjects having a BMI less than about 40 with multiple VLUs are more likely to respond to treatment with a connexin 43 modulating agent than mVLU subjects having a BMI greater than about 40 who are treated with standard of care. For example, following treatment with an anti-connexin 43 modulating agent such as a connexion 43 antisense oligonucleotide, a subject with BMI of 30 is up to 4 times or more likely to heal than one with a BMI of 43.

This invention also relates to methods of determining whether a subject is a responder subject, likely to be responsive to treatment by a connexin 43 modulating agent, based on indicators described herein, for example. In some embodiments, this invention relates to a method of determining whether a patient is a responder to treatment with a connexin 43 modulating agent, the method comprising determining one or more subject indicators selected from the group of a subject's VLU status (single or multiple VLUs), age, and BMI measurement, and, optionally, percent healing during a run-in period with, e.g., compression bandaging alone, and determining whether the indicators predict a likelihood of response to treatment with a connexin 43 modulator and treating those subjects expected or predicted to respond to treatment. In some embodiments, the method of determining whether a subject is a responder can be used in conjunction with any of the methods of treatment and uses described herein. As discussed herein, mVLU subjects have been surprisingly discovered to be more likely to heal following treatment with a connexin 43 modulating agent than patients who do not meet the criteria for responsiveness to treatment, according to indicators such as those set forth herein.

The invention relates in some aspects to pharmaceutical formulations and packages and kits including a pharmaceutical formulation comprising a pharmaceutically acceptable carrier, and a pharmaceutically acceptable anti-connexin modulating agent, for administering to responsive subjects with mVLUs an anti-connexin modulating agent, in amounts effective to promote mVLU healing in a subject. The package optionally comprises a label and/or instructions for this use.

In some embodiments, the formulations of this invention for use in treating mVLUs, mDFUs, or other resistant lesions may comprise a connexin 43 modulating agent and one or more pharmaceutically acceptable vehicles formulated for topical administration. In some embodiments the composition is formulated to provide sustained release of the connexin 43 modulating agent.

The terms “modulating agent,” “modulator” and “modulation” of connexin protein activity, as used herein in its various forms, refers to inhibition in whole or in part of the expression, action or activity of a connexin or a connexin hemichannel or connexin gap junction, in whole or in part, and may function as anti-connexin agents, including as gap junction modulation agents. In some embodiments the connexin protein modulating agents of this invention include anti-connexin 43, 30 or 26 oligonucleotides, anti-connexin 43, 30 or 26 peptides, anti-connexin 43, 30 or 26 peptidomimetics, or gap junction closing compounds, hemichannel closing compounds, and connexin carboxy-terminal polypeptides useful for healing wounds on subjects with more than one resistant wound, e.g., more than one VLU on one or both legs.

The polynucleotides of this invention include synthesized polynucleotides having a length of less than 80 nucleotides, e.g., from 12-18 to about 50-80 nucleotides, preferably about 30 nucleotides or less, e.g., from 12 to about 30 nucleotides, and more preferably from about 15 to about 30 nucleotides. In one example, the polynucleotide has 30 nucleotides.

Such formulations include, for example, topical delivery forms and formulations. Such delivery forms and formulations include those for the treatment of a subject as disclosed herein. In some embodiments the anti-connexin polynucleotides are anti-connexin 43 oligonucleotides (ODN). In other embodiments, the connexin protein modulating compounds are anti-connexin 43, 30 or 26 peptides or peptidomimetics, e.g., anti-connexin 43, 30 or 26 hemichannel blocking peptides or anti-connexin 43 hemichannel blocking peptidomimetics. In some embodiments the gap junction closing compounds and hemichannel closing compounds are connexin 43, 30 or 26 gap junction closing compounds and connexin 43, 30 or 26 hemichannel closing compounds. Preferred connexin carboxy-terminal polypeptides are connexin 43, 30 or 26 carboxy-terminal polypeptides. Treatment of a subject, e.g., for mVLUs, with one or more pharmaceutical compositions of the invention, e.g., an anti-connexin ODN and a connexin hemichannel blocking agent, e.g., a peptide or peptidomimetic, or a first anti-connexin agent and a second anti-connexin agent, may comprise their simultaneous, separate, sequential or sustained administration.

The pharmaceutical formulations of this invention may further comprise one or more pharmaceutically acceptable excipients. In some embodiments the formulation may comprise a connexin 43, 30 or 26 antisense oligonucleotides. The connexin 43 antisense oligonucleotide that are included in the formulation may be, in some embodiments, an unmodified connexin 43 antisense oligodeoxynucleotide. In some embodiments the vehicle may be or contain a gel, a poloxamer (liquid or gel), a carboxycellulose (e.g. carboxymethylcellulose), a collagen (e.g., a Type I collagen), a collagenous material comprising tropocollagen, a hyaluronan or derived-hyaluronic acid, and/or an oil (e.g., Emu oil). The formulations of this invention do not comprise the connexin 43 modulating agent in sterile water as the only vehicle.

In some embodiments, the pharmaceutically acceptable carrier or vehicle is, or comprises, a gel. In one aspect the gel can be a reverse-thermosetting gel which is a liquid at low temperatures, for example at 2-8° C., and which undergoes a reversible liquid to gel transition at temperatures greater than approximately 15° C. Thus, in some embodiments the carrier may be a liquid at temperatures below approximately 15° C., but may form a gel at temperatures above approximately 15° C., such as room temperature or at body temperature. In some instances, the gel is a nonionic polyoxyethylene-polyoxypropylene copolymer gel. In some embodiments the gel is a pluronic gel. The pluronic gel may be, for example, poloxamer 407, also sometimes referred to as Pluronic F-127 (BASF). In some embodiments, the formulations of this invention may comprise from about 15 to about 30% (w/v) gel. In some embodiments, the formulations of this invention may comprise from about 20 to about 25% (w/v) gel. In some embodiments, the formulations of this invention may comprise about 22.6% (w/v) poloxamer 407 gel.

In some embodiments, treatment with a connexin protein modulating agent is administered in conjunction with compression bandaging, off-loading, or other standard-of-care therapy. Exemplary connexin protein modulating agents include connexin 43, 30 or 26 modulating agents.

It has also been surprisingly discovered that connexin 43 levels measured in the dermis and/or epidermis at the edges of multiple VLUs in humans appear higher than connexin 43 levels measured at the edges of single VLUs in humans. Accordingly, a determination of high connexin 43 levels in the dermis or epidermis of a resistant wound may be used as a method of diagnosing a resistant lesion and/or responder patient prior to prescribing treatment with a connexin 43 modulating agent.

Subjects with mVLUs, mDFUs, or other resistant lesions or otherwise assessed to have a likelihood of response to treatment of by a connexin protein modulating agent, according to the methods of this invention, are also referred to as “responder” subjects. By likelihood of response is meant a likelihood that resistant lesion healing is promoted with a connexin protein modulating agent over, for example, it may be promoted treatment with standard of care (e.g., compression bandageing or off-loading) and/or vehicle by at least a factor of 2 when compared to treatment of lesions on mVLU subjects or subjects with other resistant lesions with standard of care, such as treatment by compression bandaging. The probability of a resistant wound on a responder subject healing with treatment with a connexin protein modulating agent is likely to be at least about 10% to 15% higher than treatment with standard of care (e.g., compression bandageing or off-loading) and/or vehicle. In other words, the healing delta between responder subjects treated with a connexin protein modulating agent will be at least about 10% to 15%. Typically this delta will be 20% or more, and can be 25%, 30%, 35%, 40% and 45% or more.

Resistant lesions or wounds include multiple VLUs, multiple diabetic foot ulcers (DFUs), multiple pressure ulcers, and those with relatively few signs of healing during a screening period with standard therapy, e.g., compression bandaging therapy in the case of VLUs. In some aspects resistant lesions may also be characterized by less granulation and epithelialization during the screening or pretreatment period, or at the time of treatment with the connexin 43 modulating agent. The screening or pretreatment period may be from about 10 days to about 1-4 weeks, for example, and is typically 2 weeks, 3 weeks or 4 weeks, but may be longer. A 2-week screening or pretreatment period is common.

Still another factor includes the amount healing during a pretreatment or run-in period with standard-of-care treatment, and the amount healing during a pretreatment or run-in period with standard-of-care treatment together with a hydrogel or other agent used to maintain a moist wound environment, as measured by, for example, linear lesion advance. Yet other factors include the size and/or duration of the lesion. In one aspect, in the case of VLUs, the indicator is the presence of mVLUs, as noted. A resistant lesion is any lesion exhibiting one or more indicators of a resistant lesion, as described herein. A resistant lesion may exhibit, for example, one or more of the indicators based on the baseline area of lesion, the % epithelialization of the lesion, and baseline circumference, as described herein. In addition, a lesion may be characterized as a resistant lesion if it is present on a subject having hemoglobin A1c (HbA1c) levels and/or BMI, as described herein. In some instances, the resistant lesion may exhibit two, three, four, five or all six of these indicators, including the subject having an HbA1c level and BMI as described herein. Still another factor to be considered in conjunction with other factors is the amount healing during a pretreatment or run-in period with standard-of-care treatment together with a hydrogel or other agent used to maintain a moist wound environment, as measured by, surface area reduction or percent lesion surface area reduction.

In one embodiment, where the amount of healing during a pre-treatment or run-in period with standard-of-care treatment is used as one factor, together with other factors, in characterizing a resistant wound, the pretreatment or run-in period is generally long enough so that some lesions can show material or art-recognized advancement to resolution with standard treatment. In one embodiment, the period is generally up to 2-4 weeks, but can be longer. Where a pretreatment or run-in period of about 2 weeks is utilized as one factor in conjunction with other factors to determine whether the subject has a resistant lesion, i.e., a period where the patient is treated with standard-of-care such as compression bandaging and/or off-loading, for example, if the treated lesion(s) on the patient does not increase by more than 1, 2, 3, 4, 5, 6, 7, 8, 9, 10, 11, 12, 13, 14, or 15%, or heal by more than about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30% (in percent surface area reduction of the lesion), or by any range between any two recited values, or any percentage in between any two recited values. For example, the treated lesion may not heal by more than about 25%, more than about 20-30%, or more than about 25-30%, preferably by less than about 25%, and more preferably by less than about 20%. In other embodiments, the lesion does not heal by more than about 10-20%, preferably less than from about 10-15%, for example less than about 17.5%. Where a pretreatment or run-in period of about 4 weeks is utilized to determine whether the subject has a persistent lesion, if the treated lesion(s) on the patient does not increase by more than about 15% or heal by more than about 30-50% or 30-40% surface area reduction, preferably less than about 35%, and more preferably less than about 30%, the lesion is a resistant lesion for purposes of this invention. In still other embodiments, the lesion does not show a surface area reduction of at least about 20% over a 2- to 4-week pretreatment or run-in period during with the subject is treated with a hydrogel or other agent to maintain a moist wound environment (for example, Curasol Hydrogel, Gentell Hydrogel, poloxamer gel, or any other acceptable hydrogel or moistening agent for treating lesions) plus standard-of-care (for example, compression and/or off-loading, etc.). These amounts of healing can be used in combination with other factors to determine if a wound is a resistant wound.

In some embodiments, the lesion is identified as a resistant lesion treatment with an anti-connexin 43 modulating agent if the lesion(s) to be treated is >about 5.0, 5.1, 5.2, 5.3, 5.4, 5.4, 5.5, 5.6, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.0 or >about 10.0 cm² (size). In some embodiments, the lesion is identified as a resistant lesion treatment with an anti-connexin 43 modulating agent if the lesion(s) to be treated is >5 cm², preferably >6.0 cm², more preferably >7.0 cm² or >8.0 cm², or also preferably >8.6 cm².

In some embodiments, the lesion is identified as a resistant lesion treatment with an anti-connexin 43 modulating agent if the lesion (s) to be treated has minimal epilethialization, that is, epilethialization of less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, 5.1, 5.2, 5.3, 5.4, 5.5, 5.6, 5.7, 5.8, 5.8, 5.9, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0% of the surface area of the wound. In some embodiments, the lesion is identified as a resistant lesion treatment with an anti-connexin 43 modulating agent if the wound(s) to be treated has epilethialization of less than about 30%, 20%, 10% or less, preferably 5.0% or less, more preferably 2.5% or less.

In some embodiments, the lesion is identified as a resistant lesion treatment with an anti-connexin 43 modulating agent if the subject to be treated has a HbA1c of greater than about 6.0, 6.1, 6.2, 6.3, 6.4, 6.5, 6.6, 6.7, 6.8, 6.9, or about 7.0%, or greater than any range between any two recited values, or greater than any value in between any two recited values. In some embodiments, the lesion is identified as a resistant lesion treatment with an anti-connexin 43 modulating agent if the subject to be treated has a HbA1c of greater than about 6.5%. In some embodiments, the lesion is identified as a resistant lesion treatment with an anti-connexin 43 modulating agent if the subject to be treated has a HbA1c of greater than 6.0%, 6.5% or greater than 7.0%.

In some embodiments, the lesion is identified as a resistant lesion treatment with an anti-connexin 43 modulating agent if the lesion(s) to be treated has a circumference of less than about 4.5, 4.6, 4.7, 4.8, 4.9, 5.0, 5.1, 5.2, 5.3, 5.4, 5.5, 5.6, 5.7, 5.8, 5.8, 5.9, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or <about 12.0 cm, or less than any range between any two recited values, or less than any value in between any two recited values. In some embodiments, the resistant lesion having a circumference less than the recited length may also have a relatively convex circumference. In some embodiments, the lesion is identified as a resistant lesion treatment with an anti-connexin 43 modulating agent if the lesion has a circumference of less than about 10.0 cm, 9.0 cm, 8.0 cm, 7.0 cm, 6.0 cm or 5.7 cm.

In other embodiments, where a pretreatment or run-in period of about 2 weeks is utilized to help determine whether the subject has a resistant lesion based on linear lesion advance (LLA), if the treated lesion (s) on the subject shows a linear lesion advance of less than about 0.002, 0.003, 0.004, 0.005, 0.006 or 0.007 cm/day or any range between any of those values (e.g., about 0.002 to about 0.0065 cm/day), or a linear lesion advance of about 0.03, 0.035, 0.04, 0.042, 0.05 cm/week, or any range between any of those values (e.g., about 0.035 to about 0.05 cm/week), the lesion is a resistant lesion for purposes of this invention. In other embodiments, where a pretreatment or run-in period of more than 2 weeks, for example up to about 4 weeks, or any period between about 2 weeks to about 4 weeks, is utilized to determine whether the subject has a persistent lesion, if the treated lesion (s) on the patient shows a linear lesion or wound advance of less than about 0.004, 0.005, 0.006, or 0.0065 cm/day or any range between any of those values (e.g., about 0.005 to about 0.0065 cm/day), or a linear lesion advance of less than about 0.03, 0.035, 0.04, 0.042, or about 0.045 cm/week, or less than about any range between any of those values (e.g., about 0.04 to about 0.045 cm/week), the lesion is a resistant lesion for purposes of this invention. In other embodiments, where a pretreatment or run-in period of about 2 weeks or about 4 weeks is utilized to determine whether the subject has a LLA-resistant lesion, if the treated lesion on the patient shows a linear wound advance of 0.050 cm/week or less, the lesion is an LLA resistant lesion for the purposes of this invention

In some embodiments, the lesion is identified as a resistant lesion for treatment with an anti-connexin 43 modulating agent if the lesion(s) to be treated is >about 8.5 cm² (size) and a duration of more than 6 months, a minimal degree of epitheliazation, and/or hemoglobin A1c (HbA1c) of 6.5% or greater.

In one embodiment, the lesion is identified as a resistant lesion for treatment with an anti-connexin 43 modulating agent if the lesion to be treated is present on a subject having a BMI of less than about 40, 39, 38, 37, 36, 35, 34, 33, 32, 31, 30, 29, 28, 27, 26, or about 25. In some embodiments, the lesion is identified as a resistant lesion for treatment with an anti-connexin 43 modulating agent if the lesion to be treated is present on a subject having a BMI of less than about 40, preferably less than 35 or 30, and most preferably less than 25.

In yet other embodiments, the wound is identified as a resistant lesion for treatment with an anti-connexin protein modulating agent if the lesion (s) to be treated shows one or more of the following: (a) low levels of mitotic activity or fewer cells; (b) high levels of cytokines and/or proteases or other markers or marker ratios and panels indicative of resistant lesions; (c) low levels of growth factors; and/or (d) fibroblast senescence, in comparison to healing or acute wounds.

In one embodiment, the responder patient has one or more of the following characteristics: (1) multiple venous leg ulcers on one or both legs; (2) age equal to 50-52 years of age; (3) BMI less than about 40-42; and, (4) healing by less than about 30-40% during a pretreatment period or a run-in period with standard of care treatment (e.g., compression bandaging). In other embodiments, this invention also relates to methods of determining whether subjects are likely to respond to treatment by a connexin 43 modulating agent based on indicators described herein. Methods of assessing whether a subject is a likely responder subject can also be used in conjunction with the methods of treatment and uses described herein.

Other indicators of resistant lesions or wounds include biomarkers of resistant lesions as described herein. With respect to markers, resistant lesion fluids show, for example, lower ratios of two key cytokines, TNFα and IL-1, and their natural inhibitors, P55 and IL-1 receptor antagonist. Resistant lesions will show from about 1:1 to about 5:1 in the case of P55/TNFα and/or from about 1:1 to about 10:1 in the case of IL-RA/IL-1. Resistant wounds will also show high levels of cytokines such as IL-1, IL-6 and TNFα in fluids collected from the lesions. In other embodiments, a resistant lesion is identified by evaluating the change in levels of cytokines over, for example, a 2-4 week pretreatment period during which the levels of cytokines are not significantly decreased.

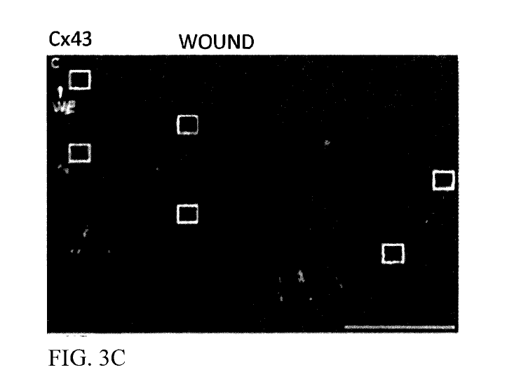

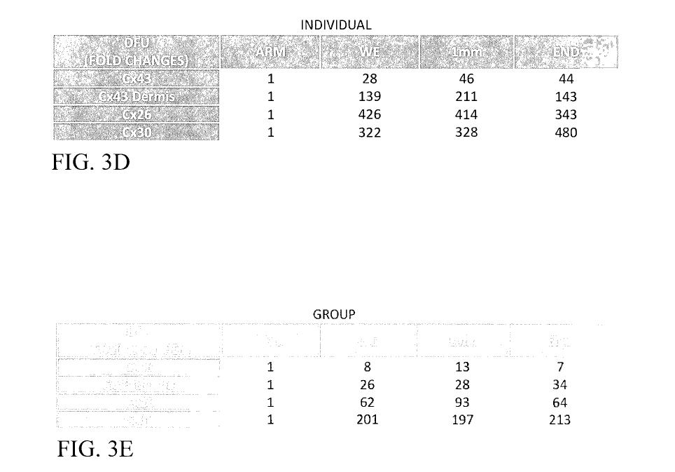

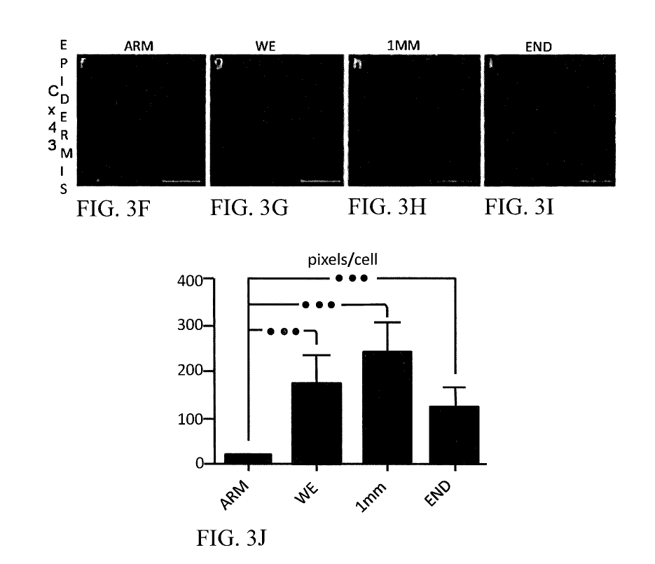

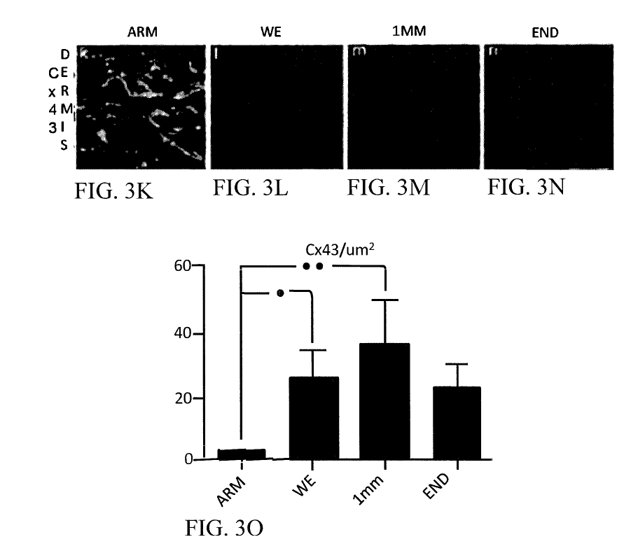

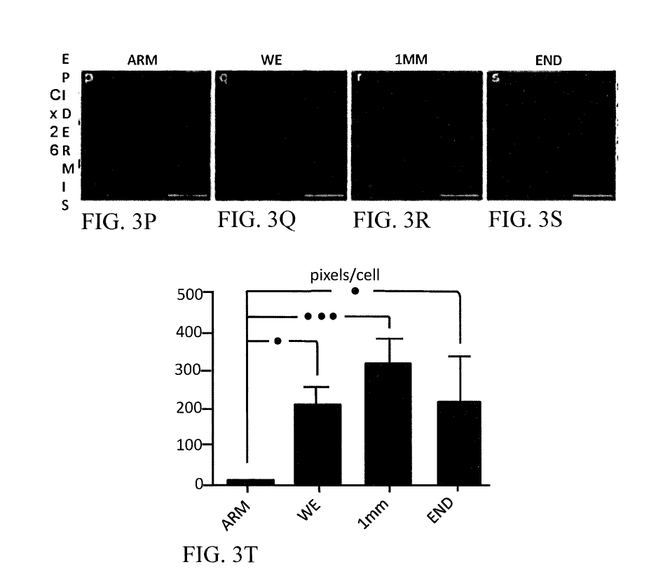

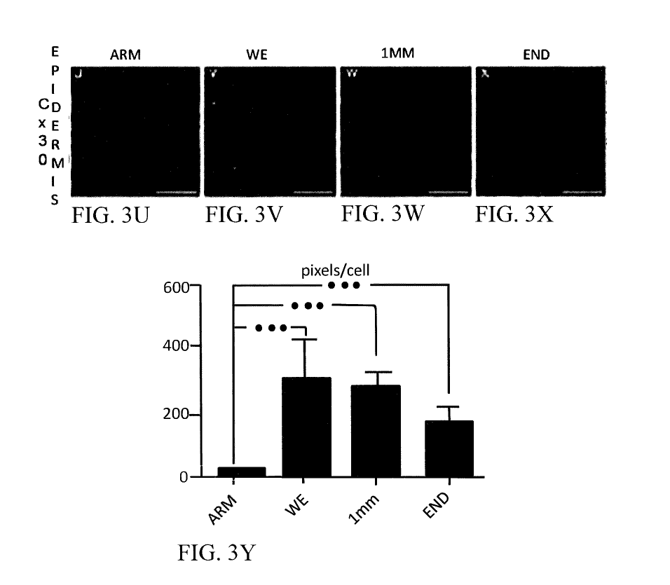

Other resistant lesions are those that show significantly elevated levels of proteases compared to acute wounds. The average level of protease activity in chronic wound fluids (87 μg collagenase equivalents/ml) is about 100-fold higher than in mastectomy fluids. Also, the range of protease activity in chronic wound fluids is rather large (from 1 to 584 μg collagenase equivalents/ml). More importantly, the levels of protease activity tend to decrease in chronic venous ulcers 2 weeks after the ulcers begin to heal (FIG. 3). In some embodiments the protease may be, for example, a metalloproteinase.

In still other embodiments, a resistant wound will contain high levels of IL1, IL6, and matrix metalloproteinases (MMPs), and an abnormally high MMP to TIMP ratio. MMPs are part of the larger family of metalloproteinase enzymes that play an important role in wound healing. In normal wound healing, MMPs are produced by activated cells (neutrophils and macrophages) and wound cells (epithelial cells, fibroblasts and vascular endothelial cells). The MMPs are inhibited by specific endogenous tissue inhibitor of metalloproteinases, which comprise a family of four protease inhibitors: TIMP-1, TIMP-2, TIMP-3, and TIMP-4. As an example, elevated levels of MMP-2, MMP-8 and/or MMP-9, preferably elevated levels of MMP-9 may characterize a resistant wound.

In some embodiments, a resistant wound may also be characterized by low levels of TGFβ and/or low levels of one or more MMP tissue inhibitors (TIMP), for example TIMP-1 or TIMP-2). Resistant wounds may also be characterized by the MMP-9:TIMP-2 ratio. In some embodiments, the resistant wound may be characterized by increases in one or more of IL-1, IL-6, IL-8, MIP-1α, TNFα and/or IL-1β or any combination thereof. Higher levels of IL-1α, IL-1β, IFNγ, IL-12p40, GM-CSF and IL-IRA may also be present at elevated levels in resistant wounds.

Healing as used herein refers to healing based on one or more assessments for wound, or lesion, healing, including healing of a wound on an mVLU or mDFU subject, such as complete wound closure, or reduction or percent change in wound surface area.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred pet animals are dogs and cats.

As used herein, “preventing” means preventing in whole or in part, or ameliorating or controlling.

As used herein, a therapeutically effective amount of the connexin 43 modulating agent is any amount effective to promote healing of a resistant lesion in a subject. For example, a therapeutically effective amount of the connexin 43 modulating agent when used to treat mVLUs is the amount effective to promote healing of mVLUs.

The terms “peptidomimetic” and “mimetic” include synthetic or genetically engineered chemical compounds that may have substantially the same structural and functional characteristics of protein regions which they mimic. In the case of connexins, these may mimic, for example, the extracellular loops of opposing connexins involved in connexon-connexon docking and cell-cell channel formation, and/or the extracellular loops of hemichannel connexins.

As used herein, the term “peptide analogs” refer to the compounds with properties analogous to those of the template peptide and can be non-peptide drugs. “Peptidomimetics” (also known as peptide mimetics) which include peptide-based compounds, also include such non-peptide based compounds such as peptide analogs. Peptidomimetics that are structurally similar to therapeutically useful peptides can be used to produce an equivalent or enhanced therapeutic or prophylactic effect. Generally, peptidomimetics are structural or functional mimics (e.g., identical or similar) to a paradigm polypeptide (i.e., a polypeptide that has a biological or pharmacological function or activity), but can also have one or more peptide linkages optionally replaced by a linkage selected from the group consisting of, for example, —CH2NH—, —CH2S—, —CH2-CH2-, —CH═CH— (cis and trans), —COCH2-, —CH(OH)CH2-, and —CH2SO—. The mimetic can be either entirely composed of natural amino acids, synthetic chemical compounds, non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. In the case of connexins, these can mimic, for example, the extracellular loops of opposing connexins involved in connexon-connexon docking and cell-cell channel formation. For example, a mimetic composition can be useful as a gap junction modulating agent if it is capable of down-regulating biological actions or activities of connexons, such as, for example, preventing the docking of connexons to form gap-junction-mediated cell-cell communications, or preventing the opening of connexons to expose the cell cytoplasm to the extracellular millieu. Peptidomimetics encompass those described herein, as well as those as may be known in the art, whether now known or later developed.

The term “wound dressing” or “lesion dressing” refers to a dressing for topical application to a resistant lesion or wound and excludes compositions suitable for systemic administration. For example, the one or more anti-connexin 43, anti-connexin 30 or anti-connexin 26 agents, including gap junction modulation agents, may be dispersed in or on a solid sheet of lesion contacting material such as a woven or nonwoven textile material, or may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example in a gel or ointment. In certain embodiments the one or more anti-connexin agents, including gap junction modulation agents are dispersed in or on a biodegradable sheet material that provides sustained release of the active ingredients into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

As used herein, “matrix” includes for example, matrices such as collagen, acellular matrices, crosslinked biological scaffold molecules, tissue-based matrices (including pig-based wound healing matrices), cultured epidermal autografts, cultured epidermal allografts, tissue-engineered skin, collagen and glycosaminoglycan dermal matrices inoculated with autologous fibroblasts and keratinocytes, Alloderm (a nonliving allogeneic acellular dermal matrix with intact basement membrane complex), living skin equivalents (e.g., Dermagraft (living allogeneic dermal fibroblasts grown on degradable scaffold), TransCyte (an extracellular matrix generated by allogeneic human dermal fibroblasts), Apligraf (a living allogeneic bilayered construct containing keratinocytes, fibroblasts and bovine type I collagen), and OrCel (allogeneic fibroblasts and keratinocytes seeded in opposite sides of bilayered matrix of bovine collagen), animal derived dressings (e.g., Oasis's porcine small intestinal submucosa acellular collagen matrix; and E-Z Derm's acellular xenogeneic collagen matrix), tissue-based bioengineered structural frameworks, biomanufactured bioprostheses, and other implanted or applied structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson et al., Trans Am Soc Artiflntern 1990; 36:340-343) or other biomatrix which have been engineered to reduce the antigenic response to the xenograft material. Other matrix useful in promotion of resistant wound healing may include for example, processed bovine pericardium proteins comprising insoluble collagen and elastin (Courtman et al., J Biomed Mater Res 1994; 28:655-666) and other acellular tissue which may be useful for providing a natural microenvironment for host cell migration to accelerate tissue regeneration (Malone et al., J Vasc Surg 1984; 1:181-91). In certain embodiments, the matrix material may be supplemented with one or more anti-connexin 43 modulating agents, such as anti-connexin 43 polynucleotides and/or the one or more anti-connexin 43 peptides or peptidomimetics for site specific release of such agents.

As used herein, “resistant lesion promoting matrix” includes for example, synthetic or naturally occurring matrices such as collagen, acellular matrix, crosslinked biological scaffold molecules, tissue based bioengineered structural framework, and other structures useful in the promotion of resistant wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson G J et al. (1990) Trans Am Soc Artif Intern 36:340-343) or other biomatrix which have been engineered to reduce the antigenic response to the xenograft material. Other matrices useful in promotion of wound healing may include for example, proteins comprising insoluble collagen and elastin (Courtman D W et al. (1994) J Biomed Mater Res 28:655-666) and other acellular tissue which may be useful for providing a natural microenvironment for host cell migration to accelerate epilethialization (Malone J M et al. (1984) J Vasc Surg 1:181-91). The invention contemplates a synthetic or natural matrix comprising one or more anti-connexin protein agents.

In one embodiment, the formulations of this invention also include salts of connexin polynucleotides, including for example sodium salts, potassium salts or any other salt suitable for topical administration.

### Connexin Protein Anti-Connexin Agents

Anti-connexin protein agents, or connexin modulating agents, of the invention described herein are capable of modulating or affecting the transport of molecules into and out of cells (e.g., blocking or inhibiting or downregulating), and modulating cellular communication (e.g., cell to cell). The anti-connexin protein agents include, for example, anti-connexin 43, anti-connexin 30 or anti-connexin 26 agents. Thus, certain anti-connexin protein agents described herein are capable of blocking or inhibiting the transport of molecules into and out of cells. Thus certain anti-connexin agents described herein modulate cellular communication (e.g. cell to cell). Certain anti-connexin agents affect transmission of molecules between the cell cytoplasm and the periplasmic or extracellular space. Such agents are generally targeted to hemichannels (also called connexons), which may be independently involved in the exchange of small molecules between the cell cytoplasm and an extracellular space or tissue. Thus, a compound provided herein may directly or indirectly reduce coupling between cells (via gap junctions) or between a cell and an extracellular space or tissue (via hemichannels), and the modulation of transport of molecules from a cell into an extracellular space is within the scope of certain compounds and embodiments of the invention.

Any anti-connexin protein agent that is capable of eliciting a desired inhibition of the passage (e.g. transport) of molecules through a gap junction or connexin hemichannel may be used in embodiments of the invention. Any anti-connexin 43 agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm). Such anti-connexin 43 agents may modulate the passage of molecules through a gap junction or connexin hemichannel with or without gap junction uncoupling (blocking the transport of molecules through gap junctions). Such compounds include, for example, binding proteins, polypeptides, and other organic compounds that can, for example, block the function or activity of a gap junction or a hemichannel in whole or in part.

Certain anti-connexin protein agents, such as anti-connexin 43 agents, provide downregulation of connexin expression (for example, by downregulation of mRNA transcription or translation) or otherwise decrease or inhibit the activity of the connexin protein, connexin hemichannels or gap junctions. In the case of downregulation, this will have the effect of reducing direct cell-cell communication by gap junctions, or exposure of cell cytoplasm to the extracellular space by hemichannels, at the site at which connexin expression is downregulated.

As used herein, “anti-connexin protein agent” or “connexin modulating agent” may include those agents or compounds that prevent, decrease or modulate, in whole or in part, the activity, function, or formation of a hemichannel or a gap junction. In certain embodiments, a gap junction modulation agent prevents or decreases, in whole or in part, the function of a hemichannel or a gap junction. In certain embodiments, a gap junction modulation agent induces closure, in whole or in part, of a hemichannel or a gap junction. In other embodiments, a gap junction modulation agent blocks, in whole or in part, a hemichannel or a gap junction. In certain embodiments, a gap junction modulation agent decreases or prevents, in whole or in part, the opening of a hemichannel or gap junction. In certain embodiments, said blocking or closure of a gap junction or hemichannel by a gap junction modulation agent can reduce or inhibit extracellular hemichannel communication by preventing or decreasing the flow of small molecules through an open channel to and from an extracellular or periplamic space. Peptidomimetics, and gap junction phosphorylation compounds that block hemichannel and/or gap junction opening are presently preferred. In some embodiments the anti-connexin protein agent may be an anti-connexin 43 agent, an anti-connexin 30 agent, or an anti-connexin 26 agent.

In certain embodiments, an anti-connexin agent prevents, decreases or alters the activity or function of a hemichannel or a gap junction. As used herein, modulation of the gap junction activity or function by the anti-connexin agent may include the closing of gap junctions, closing of hemichannels, and/or passage of molecules or ions through gap junctions and/or hemichannels.

Examples of anti-connexin protein agents include agents that decrease or inhibit expression or function of connexin protein mRNA and/or protein or that decrease activity, expression or formation of connexin protein, connexin hemichannels gap junctions. As an examples, an anti-connexin protein agents include anti-connexin 43 agents that decrease or inhibit expression or function of connexin 43 mRNA and/or protein or that decrease activity, expression or formation of connexin 43, connexin hemichannels gap junctions. Anti-connexin protein agents include anti-connexin protein polynucleotides, such as antisense protein polynucleotides, such as anti-connexin protein oligonucleotides, connexin protein oligodeoxynucleotides and other polynucleotides (such as polynucleotides having siRNA or ribozyme functionalities), as well as antibodies and binding fragments thereof that bind connexin protein, and anti-connexin protein peptides and polypeptides, including peptidomimetics and peptide analogs of connexin that modulate hemichannel or gap junction activity or function, and other gap junction blocking agents and gap junction protein phosphorylating agents. Anti-connexin protein peptides and polypeptides may, for example, bind to connexin protein to inhibit its function, or may inhibit connexin 43 function by mimicking regions of connexin protein to inhibit or disrupt its binding to other gap junction proteins. The agents may be anti-connexin 43 agents, anti-connexin 30 agents and/or anti-connexin 26 agents.

### Anti-Connexin Protein Polynucleotides

Anti-connexin polynucleotides include connexin antisense protein polynucleotides as well as polynucleotides which have functionalities which enable them to downregulate connexin protein expression, such as connexin 43 expression. Other suitable anti-connexin 43, 30 or 26 polynucleotides include anti-connexin protein oligonucleotides, connexin protein oligodeoxynucleotides, connexin protein RNAi polynucleotides and connexin protein siRNA polynucleotides.

Synthesis of antisense polynucleotides and other anti-connexin 43 polynucleotides such as RNAi, siRNA, and ribozyme polynucleotides as well as polynucleotides having modified and mixed backbones can be performed by any suitable method. See e.g. Stein C. A. and Krieg A. M. (eds), Applied Antisense Oligonucleotide Technology, 1998 (Wiley-Liss). Methods of synthesizing antibodies and binding fragments as well as peptides and polypeptides, including peptidomimetics and peptide analogs can also be performed using suitable methods. See e.g. Lihu Yang et al., Proc. Natl. Acad. Sci. U.S.A., 1; 95(18): 10836-10841 (Sep. 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) may target the connexin 43 protein. Typically the polynucleotides are single stranded, but may be double stranded.

The antisense polynucleotide may inhibit transcription and/or translation of a connexin protein, such as connexin 43, 30 or 26. Preferably the polynucleotide is a specific inhibitor of transcription and/or translation from the connexin 43, 30 or 26 gene or mRNA, and does not inhibit transcription and/or translation from other genes or mRNAs. Screening of the polynucleotide sequence in a human genome sequence database for specificity may also be performed. The product may bind to the connexin 43, 30 or 26 gene or mRNA either (i) 5′ to the coding sequence, and/or (ii) to the coding sequence, and/or (iii) 3′ to the coding sequence.

The antisense polynucleotide is generally antisense to connexin protein mRNA, for example, connexin 43, 30 or 26 mRNA. Such a polynucleotide may be capable of hybridizing to connexin protein mRNA and may thus inhibit the expression of connexin by interfering with one or more aspects of connexin protein mRNA metabolism including transcription, mRNA processing, mRNA transport from the nucleus, translation or mRNA degradation. The antisense polynucleotide typically hybridizes to the connexin protein mRNA to form a duplex which can cause direct inhibition of translation and/or destabilization of the mRNA. Such a duplex may be susceptible to degradation by nucleases.

The antisense polynucleotide may hybridize to part of the connexin protein mRNA, such as connexin 46, 30 or 26 mRNA. Typically the antisense polynucleotide hybridizes to the ribosome binding region or the coding region of the connexin protein mRNA. The polynucleotide may be complementary to a region of the connexin mRNA. For example, the polynucleotide may be the exact complement of a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin protein mRNA under conditions of medium to high stringency such as 0.03M sodium chloride and 0.03M sodium citrate at from about 50° C. to about 60° C.

For certain aspects, the polynucleotides of this invention include synthesized polynucleotides having a length of less than 80 nucleotides, e.g., from 15-18 to about 50-80 nucleotides, preferably about 30 nucleotides or less, e.g., from 15 to about 30 nucleotides, and more preferably from about 15 to about 20 nucleotides. In one example, the polynucleotide has 30 nucleotides.

Alternatively, the antisense polynucleotides may be part of compositions which may comprise polynucleotides to more than one connexin protein. Preferably, the connexin protein to which polynucleotides are directed is connexin 43. Other connexin proteins to which oligodeoxynucleotides are directed may include, for example, connexins 26, 30, 30.3, 31.1, 32, 36, 37, 40, 40.1, 45, and 46.6. Suitable exemplary polynucleotides (and ODNs) directed to various connexins are set forth in Table 1.

The polynucleotides for use in the invention may suitably be unmodified phosphodiester oligomers. Such oligodeoxynucleotides may vary in length. A 30 mer polynucleotide has been found to be particularly suitable.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin 43 antisense polynucleotides can include polynucleotides such as oligodeoxynucleotides selected from SEQ ID NO: 1-3 set forth in Table 1: Suitable polynucleotides for the preparation of the combined polynucleotide compositions described herein, for combination with the connexin 43 modulating agent include polynucleotides for connexins 26, 30, 31.1, 32 and 37 are also described in Table 1.

TABLE 1

| | | |
|---|---|---|
| 5′ GTA ATT GCG GCA AGA AGA ATT GTT TCT GTC 3′ | (connexin 43) | (SEQ. ID. NO: 1) |
| 5′ GTA ATT GCG GCA GGA GGA ATT GTT TCT GTC 3′ | (connexin 43) | (SEQ. ID. NO: 2) |
| 5′ GGC AAG AGA CAC CAA AGA CAC TAC CAG CAT 3′ | (connexin 43) | (SEQ. ID. NO: 3) |
| 5′ TCC TGA GCA ATA CCT AAC GAA CAA ATA 3′ | (connexin 26) | (SEQ. ID. NO: 21) |
| 5′ CTC AGA TAG TGG CCA GAA TGC 3′ | (connexin 30) | (SEQ. ID. NO: 22) |
| 5′ TTG TCC AGG TGA CTC CAA GG 3′ | (connexin 30) | (SEQ. ID. NO: 23 |

Although the precise sequence of the antisense polynucleotide used in the invention will depend upon the target connexin protein, for connexin 43, antisense polynucleotides having any of SEQ. ID. NO: 1-2, SEQ. ID. NO.21 or SEQ. ID. NO.22-23 have been found to be particularly suitable:

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

### Polynucleotide Homologues

Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

In one embodiment, the connexin 43 polynucleotides for use in the pharmaceutical formulations of this invention are screened against other human genome sequences to assess or determine specificity.

The invention also includes in one aspect pharmaceutical compositions with instructions for treating responder subjects having mVLUs. In one embodiment the anti-connexin 43 modulating agent is a polynucleotide. In some embodiments the anti-connexin 43 modulating agent is an oligonucleotide. The oligonucleotide may be an anti-connexin 43 antisense oligodeoxynucleotide. In one aspect, the polynucleotides of this invention may be modified or unmodified.

The invention also includes a package or kit comprising a pharmaceutical composition comprising a pharmaceutically acceptable carrier and a pharmaceutically acceptable anti-connexin modulating agent, together with a label and/or instructions for administering the composition to one or more wounds on subjects with resistant lesions such as mVLUs, where the subject is susceptible to treatment with an anti-connexin modulating agent, and the agent is administered in amounts effective to promote healing of the lesions in a subject, alone or together with compression bandaging. In one embodiment, the invention includes a package or kit comprising a pharmaceutical composition including a pharmaceutically acceptable carrier and a pharmaceutically acceptable anti-connexin 43 modulating agent, such as an anti-connexin 43 oligonucleotide, optionally with a label and/or instructions for administering the composition to responder subjects with mVLUs in amounts effective to promote mVLU healing in a subject, alone or under compression bandaging.

In one aspect the pharmaceutical formulations of this invention comprise an unmodified oligonucleotide specific to connexin 43 mRNA having the sequence 5′-GTAATTGCGGCAAGAAGAATITGITCTGTC-3′ (SEQ ID NO:1). In one aspect the olignonucleotide may be a deoxyoligonucleotide. In another aspect the oligonucleotide is chemically modified to increase half-life.

In some aspects the formulations of this invention may be formulated as a sterile, non-preserved, buffered gel at physiological pH of between pH 6.0 and 8.0, for example, pH 7.4, containing a deoxyoligonucletoide having, for example, SEQ ID NO: 1. The formulation may also contain other to maintain physiological salt concentrations, such as potassium phosphate, sodium phosphate and water-for-injection.

### Peptide and Polypeptide Anti-Connexin Agents

Connexin 43, connexin 30 or connexin 26 binding proteins, including peptides, peptidomimetics, antibodies, antibody fragments, and the like, are also suitable modulators of gap junctions and hemichannels.

Anti-connexin protein binding proteins include, for example, monoclonal antibodies, polyclonal antibodies, antibody fragments (including, for example, Fab, F(ab′)2 and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, hemichannel, or associated molecules).

Binding molecules will generally have a desired specificity, including but not limited to binding specificity, and desired affinity. Affinity, for example, may be a Ka of greater than or equal to about 10⁴ M-1, greater than or equal to about 10⁶ M-1, greater than or equal to about 10⁷ M-1, greater than or equal to about 10⁶ M-1. Affinities of even greater than about 10⁸ M-1 are suitable, such as affinities equal to or greater than about 10⁹ M-1, about 10¹⁰ M-1, about 10¹¹ M-1, and about 10¹² M-1. Affinities of binding proteins according to the present invention can be readily determined using conventional techniques, for example those described by Scatchard et al., (1949) Ann. N.Y. Acad. Sci. 51: 660.

Exemplary gap junction modulation agents may include, without limitation, polypeptides (e.g. peptiditomimetics, antibodies, binding fragments thereof, and synthetic constructs), and other gap junction blocking agents, and gap junction protein phosphorylating agents. Exemplary compounds used for closing gap junctions (e.g. phosphorylating connexin 43 tyrosine residue) have been reported in U.S. Pat. No. 7,153,822 to Jensen et al., U.S. Pat. No. 7,250,397, and assorted patent publications. Exemplary peptides and peptidomimetics are reported in Green et al., WO2006134494. See also Gourdie et al. see WO2006069181, and Tudor et al., see WO2003032964.

By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. J Cell Biol 107: 1817-1824 (1988); Meyer R. A., J Cell Biol 119: 179-189 (1992).

The extracellular domains of a hemichannel contributed by two adjacent cells “dock” with each other to form complete gap junction channels. Reagents that interfere with the interactions of these extracellular domains can impair cell-to-cell communication. Peptide inhibitors of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). Short peptides corresponding to sequences within the extracellular loops of connexins were said to inhibit intercellular communication. Boitano S. and Evans W. Am J Physiol Lung Cell Mol Physiol 279: L623-L630 (2000). The use of peptides as inhibitors of cell-cell channel formation produced by connexin (Cx) 32 expressed in paired Xenopus oocytes has also been reported. Dahl G, et al., Biophys J 67: 1816-1822 (1994). Berthoud, V. M. and Seul, K. H., summarized some of these results. Am J., Physiol. Lung Cell Mol. Physiol. 279: L619-L622 (2000).

Anti-connexin agents include peptides comprising an amino acid sequence corresponding to a transmembrane region (e.g. 1st to 4th) of a connexin (e.g. 43, 26, 30). Anti-connexin agents may comprise a peptide comprising an amino acid sequence corresponding to a portion of a transmembrane region of a connexin 43.

Anti-connexin agents include peptides having an amino acid sequence that comprises about 5 to 20 contiguous amino acids of a connexin protein such as connexin 43 (SEQ. ID. NO:4), connexin 26 or connexin 30, peptides having an amino acid sequence that comprises about 8 to 15 contiguous amino acids of connexin 43 (SEQ. ID. NO:4), connexin 26 or connexin 30, or peptides having an amino acid sequence that comprises about 11 to 13 contiguous amino acids of connexin 43 (SEQ. ID. NO:4), connexin 26 or connexin 30. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of connexin 43 (SEQ. ID. NO:4), connexin 26 or connexin 30. Other anti-connexin agents comprise the extracellular domains of connexin 43, 30 or 26, for example, corresponding to the amino acids at positions 37-76 and 178-208 of SEQ. ID. NO:4. Anti-connexin agents include peptides described herein, for example, agents having an amino acid sequence corresponding to the regions at positions 37-76 and 178-208 of SEQ. ID. NO:4. The peptides need not have an amino acid sequence identical to those portions of SEQ. ID. NO:4, and conservative amino acid changes may be made such that the peptides retain binding activity or functional activity. Alternatively, peptides may target regions of the connexin protein other than the extracellular domains (e.g. the portions of SEQ. ID. NO:4 not corresponding to positions 37-76 and 178-208). In one embodiment, the anti-connexin peptides have an amino acid sequence that comprises SEQ ID NO:8, SEQ ID NO:9, or SEQ ID NO: 10. Still other anti-connexin agents include connexin carboxy-terminal polypeptides.

In functional tests using (i) blockage of dye (Lucifer Yellow) uptake by cells in spinal cord slices, and (ii) prevention of oedema in spinal cord segments (using connexin 43 specific antisense as a positive control), connexin 43 peptides comprising SEQ ID NO:10, having sequences SEQ ID NO:8 and SEQ ID NO:9 (synthesised by Sigma-Genosys (Australia)), were shown to prevent and/or block and/or close the opening of the hemichannels by inhibiting dye uptake. In contrast, the level of dye uptake for slices treated with the peptides having SEQ ID NOS:5-7 ((FEVAFLLIQWI (SEQ ID NO:5), LLIQWYIGFSL (SEQ ID NO:6), SLSAVYTCKRDPCPHQ (SEQ ID NO:7)) and SEQ ID NOS: 11-14 (LGTAVESAWGDEQ (SEQ ID NO:11), QSAFRCNTQQPG (SEQ ID NO:12), QQPGCENVCYDK (SEQ ID NO: 13), and VCYDKSFPISHVR (SEQ ID NO: 14)) was comparable with control slices.

The connexin 43 peptide having SEQ ID NO:9 (which comprises SEQ ID NO:10), has also been shown to block swelling of cultured spinal cord segments compared to a peptide which does not block dye uptake (e.g., a peptide having SEQ ID NO: 13, which was used as a negative control). The lowest concentration of peptide (5 micromolar) used in those studies gave the best result (least oedema) when compared to media alone (p=0.001). The middle range 50 micromolar was somewhat less effective than the 5 micromolar concentration in repeat experiments.

Connexin 43 (SEQ ID NO. 4)

```
Connexin 43
(SEQ ID NO. 4)
Met Gly Asp Trp Ser Ala Leu Gly Lys Leu Leu Asp
1 5 10
Lys Val Gln Ala Tyr Ser Thr Ala Gly Gly Lys Val
                15 20
Trp Leu Ser Val Leu Phe Ile Phe Arg Ile Leu Leu
25 30 35
Leu Gly Thr Ala Val Glu Ser Ala Trp Gly Asp Glu
                        40 45
Gln Ser Ala Phe Arg Cys Asn Thr Gln Gln Pro Gly
        50 55 60
Cys Glu Asn Val Cys Tyr Asp Lys Ser Phe Pro Ile
                                65 70
Ser His Val Arg Phe Trp Val Leu Gln Ile Ile Phe
                75 80
Val Ser Val Pro Thr Leu Leu Tyr Leu Ala His Val
85 90 95
Phe Tyr Val Met Arg Lys Glu Glu Lys Leu Asn Lys
                        100 105
Lys Glu Glu Glu Leu Lys Val Ala Gln Thr Asp Gly
        110 115 120
Val Asn Val Asp Met His Leu Lys Gln Ile Glu Ile
                                125 130
Lys Lys Phe Lys Tyr Gly Ile Glu Glu His Gly Lys
                135 140
Val Lys Met Arg Gly Gly Leu Leu Arg Thr Tyr Ile
145 150 155
Ile Ser Ile Leu Phe Lys Ser Ile Phe Glu Val Ala
                        160 165
Phe Leu Leu Ile Gln Trp Tyr Ile Tyr Gly Phe Ser
        170 175 180
Leu Ser Ala Val Tyr Thr Cys Lys Arg Asp Pro Cys
                                185 190
Pro His Gln Val Asp Cys Phe Leu Ser Arg Pro Thr
                195 200
Glu Lys Thr Ile Phe Ile Ile Phe Met Leu Val Val
205 210 215
Ser Leu Val Ser Leu Ala Leu Asn Ile Ile Glu Leu
                        220 225
Phe Tyr Val Phe Phe Lys Gly Val Lys Asp Arg Val
        230 235 240
Lys Gly Lys Ser Asp Pro Tyr His Ala Thr Ser Gly
                                245 250
Ala Leu Ser Pro Ala Lys Asp Cys Gly Ser Gln Lys
                255 260
Tyr Ala Tyr Phe Asn Gly Cys Ser Ser Pro Thr Ala
265 270 275
Pro Leu Ser Pro Met Ser Pro Pro Gly Tyr Lys Leu
                        280 285
Val Thr Gly Asp Arg Asn Asn Ser Ser Cys Arg Asn
        290 295 300
Tyr Asn Lys Gln Ala Ser Glu Gln Asn Trp Ala Asn
                                305 310
Tyr Ser Ala Glu Gln Asn Arg Met Gly Gln Ala Gly
                315 320
Ser Thr Ile Ser Asn Ser His Ala Gln Pro Phe Asp
325 330 335
Phe Pro Asp Asp Asn Gln Asn Ser Lys Lys Leu Ala
                        340 345
Ala Gly His Glu Leu Gln Pro Leu Ala Ile Val Asp
        350 355 360
Gln Arg Pro Ser Ser Arg Ala Ser Ser Arg Ala Ser
                                365 370
Ser Arg Pro Arg Pro Asp Asp Leu Glu Ile
                375 380
```

The anti-connexin peptides, for example, anti-connexin 43, 30, or 26 peptides may comprise sequences corresponding to a portion of the connexin extracellular domains with conservative amino acid substitutions such that peptides are functionally active anti-connexin agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Exemplary peptides targeted to connexin 43 are shown below in Table 2. Ml, 2, 3 and 4 refer to the 1st to 4th transmembrane regions of the connexin 43 protein respectively. E1 and E2 refer to the first and second extracellular loops respectively.

TABLE 2
Peptidic Inhibitors of
Intercellular Communication (Cx43)

| | | |
|---|---|---|
| FEVAFLLIQWI | M3 & E2 | (SEQ. ID. NO: 5) |
| LLIQWYIGFSL | E2 | (SEQ. ID. NO: 6) |
| SLSAVYTCKRDPCPHQ | E2 | (SEQ. ID. NO: 7) |
| VDCFLSRPTEKT | E2 | (SEQ. ID. NO: 8) |
| SRPTEKTIFII | E2 & M4 | (SEQ. ID. NO: 9) |
| SRPTEKT | E2 | (SEQ. ID. NO: 10) |
| LGTAVESAWGDEQ | M1 & E1 | (SEQ. ID. NO: 11) |
| QSAFRCNTQQPG | E1 | (SEQ. ID. NO: 12) |
| QQPGCENVCYDK | E1 | (SEQ. ID. NO: 13) |
| VCYDKSFPISHVR | E1 | (SEQ. ID. NO: 14) |
| KRDPCHQVDCFLSRPTEK | E2 | (SEQ. ID. NO: 15) |

Table 3 provides the extracellular loops for connexin family members which are used to develop peptide inhibitors for use as described herein. The peptides and provided in Table 4, and fragments thereof, are used as peptide inhibitors in certain non-limiting embodiments. In other non-limiting embodiments, peptides comprising from about 8 to about 15, or from about 11 to about 13 amino contiguous amino acids of the peptides in this Table 4 are peptide inhibitors. Conservative amino acid changes may be made to the peptides or fragments thereof.

TABLE 3
Extracellular loops for connexin proteins

| | |
|---|---|
| E1 | |
| huCx26 | KEVWGDEQADFVCNTLQPGCKNVCYDHYFPISHIR |
| | (SEQ. ID. NO: 24) |
| huCx30 | QEVWGDEQEDFVCNTLQPGCKNVCYDHFFPVSHIR |
| | (SEQ. ID. NO: 25) |
| huCx43 | ESAWGDEQSAFRCNTQQPGCENVCYDKSFPISHVR |
| | (SEQ. ID. NO: 16) |
| E2 | |
| huCx26 | MYVFYVMYDGFSMQRLVKCNAWPCPNTVDCFVSRPTEKT |
| | (SEQ. ID. NO: 26) |
| huCx30 | MYVFYFLYNGYHLPWVLKCGIDPCPNLVDCFISRPTEKT |
| | (SEQ. ID. NO: 27) |
| huCx43 | LLIQWYIYGFSLSAVYTCKRDPCPHQVDCFLSRPTEKT |
| | (SEQ. ID. NO: 17) |

Table 4 provides the extracellular domain for connexin family members which may be used to develop peptide anti-connexin agents. The peptides and provided in Table 5, and fragments thereof, may also be used as peptide anti-connexin agents. Such peptides may comprise from about 8 to about 15, or from about 11 to about 13 amino contiguous amino acids of the peptide sequence in this Table 5. Conservative amino acid changes may be made to the peptides or fragments thereof.

TABLE 4
Extracellular domains

| | |
|---|---|
| Peptide | VDCFLSRPTEKT |
| | (SEQ. ID. NO: 8) |
| Peptide | SRPTEKTIFII |
| | (SEQ. ID. NO: 9) |
| Peptide | SRPTEKT |
| | (SEQ. ID. NO: 10) |
| huCx43 | LLIQWYIYGFSLSAVYTCKRDPCPHQVDCFLSRPTEKTIFII |
| | (SEQ. ID. NO: 18) |
| huCx26 | MYVFYVMYDGFSMQRLVKCNAWPCPNTVDCFVSRPTEKTVFTV |
| | (SEQ. ID. NO: 28) |
| huCx30 | YVFYFLYNGYHLPWVLKCGIDPCPNLVDCFISRPTEKTVFTI |
| | (SEQ. ID. NO: 29) |

In certain embodiments, it is preferred that certain peptide inhibitors block hemichannels without disrupting existing gap junctions. While not wishing to be bound to any particular theory or mechanism, it is also believed that certain peptidomimetics (e.g. the connexin 43 peptide inhibitor, VCYDKSFPISHVR, (SEQ. ID. NO: 14) block hemichannels without causing uncoupling of gap junctions (See Leybeart et al., Cell Commun. Adhes. 10: 251-257 (2003)), or do so in lower dose amounts.

A peptide comprising SRPTEKT (SEQ. ID. NO: 10), for example VDCFLSRPTEKT (SEQ. ID. NO: 8) or SRPTEKTIFII (SEQ. ID. NO: 9), may also be used, for example to block hemichannels without uncoupling of gap junctions. The peptide SRGGEKNVFIV (SEQ. ID. NO: 19) may be used that as a control sequence (DeVriese et al., Kidney Internat. 61: 177-185 (2002)). The peptides may be 3 or more amino acids in length.

Peptides or variants thereof, can be synthesized in vitro, e.g., by the solid phase peptide synthetic method or by enzyme-catalyzed peptide synthesis or with the aid of recombinant DNA technology. Solid phase peptide synthetic method is an established and widely used method, which is described in references such as the following: Stewart et al., (1969) Solid Phase Peptide Synthesis, W. H. Freeman Co., San Francisco; Merrifield, (1963) J. Am. Chem. Soc. 85 2149; Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp.3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

Table 5A shows the human connexin 43 cDNA sequence. The coding portion of the sequence is located at nucleotides 251-1399.

TABLE 5A
Human Connexin 43 from GenBank Accession No. NM_000165 (SEQ. ID. NO: 20)

| | |
|---|---|
| 1 | gagtcagtgg cttgaaactt ttaaaagctc tgtgctccaa gttacaaaaa agcttttacg |
| 61 | aggtatcagc acttttcttt cattaggggg aaggcgtgag gaaagtacca aacagcagcg |
| 121 | gagttttaaa ctttaaatag acaggtctga gtgcctgaac ttgccttttc attttacttc |
| 181 | atcctccaag gagttcaatc acttggcgtg acttcactac ttttaagcaa aagagtggtg |
| 241 | cccaggcaac atgggtgact ggagcgcctt aggcaaactc cttgacaagg ttcaagccta |
| 301 | ctcaactgct ggagggaagg tgtggctgtc agtacttttc attttccgaa tcctgctgct |
| 361 | ggggacagcg gttgagtcag cctggggaga tgagcagtct gcctttcgtt gtaacactca |
| 421 | gcaacctggt tgtgaaaatg tctgctatga caagtctttc ccaatctctc atgtgcgctt |
| 481 | ctgggtcctg cagatcatat ttgtgtctgt acccacactc ttgtacctgg ctcatgtgtt |
| 541 | ctatgtgatg cgaaaggaag agaaactgaa caagaaagag gaagaactca aggttgccca |
| 601 | aactgatggt gtcaatgtgg acatgcactt gaagcagatt gagataaaga agttcaagta |
| 661 | cggtattgaa gagcatggta aggtgaaaat gcgagggggg ttgctgcgaa cctacatcat |
| 721 | cagtatcctc ttcaagtcta tctttgaggt ggccttcttg ctgatccagt ggtacatcta |
| 781 | tggattcagc ttgagtgctg tttacacttg caaaagagat ccctgcccac atcaggtgga |
| 841 | ctgtttcctc tctcgcccca cggagaaaac catcttcatc atcttcatgc tggtggtgtc |
| 901 | cttggtgtcc ctggccttga atatcattga actcttctat gttttcttca agggcgttaa |
| 961 | ggatcgggtt aagggaaaga gcgaccctta ccatgcgacc agtggtgcgc tgagccctgc |
| 1021 | caaagactgt gggtctcaaa aatatgctta tttcaatggc tgctcctcac caaccgctcc |
| 1081 | cctctcgcct atgtctcctc ctgggtacaa gctggttact ggcgacagaa acaattcttc |
| 1141 | ttgccgcaat tacaacaagc aagcaagtga gcaaaactgg gctaattaca gtgcagaaca |
| 1201 | aaatcgaatg gggcaggcgg gaagcaccat ctctaactcc catgcacagc cttttgattt |
| 1261 | ccccgatgat aaccagaatt ctaaaaaact agctgctgga catgaattac agccactagc |
| 1321 | cattgtggac cagcgacctt caagcagagc cagcagtcgt gccagcagca gacctcggcc |
| 1381 | tgatgacctg gagatctaga tacaggcttg aaagcatcaa gattccactc aattgtggag |
| 1441 | aagaaaaaag gtgctgtaga aagtgcacca ggtgttaatt ttgatccggt ggaggtggta |
| 1501 | ctcaacagcc ttattcatga ggcttagaaa acacaaagac attagaatac ctaggttcac |
| 1561 | tgggggtgta tggggtagat gggtggagag ggaggggata agagaggtgc atgttggtat |
| 1621 | ttaaagtagt ggattcaaag aacttagatt ataaataaga gttccattag gtgatacata |
| 1681 | gataagggct ttttctcccc gcaaacaccc ctaagaatgg ttctgtgtat gtgaatgagc |
| 1741 | gggtggtaat tgtggctaaa tatttttgtt ttaccaagaa actgaaataa ttctggccag |
| 1801 | gaataaatac ttcctgaaca tcttaggtct tttcaacaag aaaaagacag aggattgtcc |
| 1861 | ttaagtccct gctaaaacat tccattgtta aaatttgcac tttgaaggta agctttctag |
| 1921 | gcctgaccct ccaggtgtca atggacttgt gctactatat ttttttattc ttggtatcag |
| 1981 | tttaaaattc agacaaggcc cacagaataa gattttccat gcatttgcaa atacgtatat |
| 2041 | tctttttcca tccacttgca caatatcatt accatcactt tttcatcatt cctcagctac |
| 2101 | tactcacatt catttaatgg tttctgtaaa catttttaag acagttggga tgtcacttaa |
| 2161 | catttttttt ttgagctaaa gtcagggaat caagccatgc ttaatattta acaatcactt |
| 2221 | atatgtgtgt cgaagagttt gttttgtttg tcatgtattg gtacaagcag atacagtata |
| 2281 | aactcacaaa cacagatttg aaaataatgc acatatggtg ttcaaatttg aacctttctc |
| 2341 | atggattttt gtggtgtggg ccaatatggt gtttacatta tataattcct gctgtggcaa |
| 2401 | gtaaagcaca cttttttttt ctcctaaaat gtttttccct gtgtatccta ttatggatac |
| 2461 | tggttttgtt aattatgatt ctttattttc tctccttttt ttaggatata gcagtaatgc |
| 2521 | tattactgaa atgaatttcc tttttctgaa atgtaatcat tgatgcttga atgatagaat |
| 2581 | tttagtactg taaacaggct ttagtcatta atgtgagaga cttagaaaaa atgcttagag |
| 2641 | tggactatta aatgtgccta aatgaatttt gcagtaactg gtattcttgg gttttcctac |
| 2701 | ttaatacaca gtaattcaga acttgtattc tattatgagt ttagcagtct tttggagtga |
| 2761 | ccagcaactt tgatgtttgc actaagattt tatttggaat gcaagagagg ttgaaagagg |
| 2821 | attcagtagt acacatacaa ctaatttatt tgaactatat gttgaagaca tctaccagtt |
| 2881 | tctccaaatg ccttttttaa aactcatcac agaagattgg tgaaaatgct gagtatgaca |
| 2941 | cttttcttct tgcatgcatg tcagctacat aaacagtttt gtacaatgaa aattactaat |
| 3001 | ttgtttgaca ttccatgtta aactacggtc atgttcagct tcattgcatg taatgtagac |
| 3061 | ctagtccatc agatcatgtg ttctggagag tgttctttat tcaataaagt tttaatttag |
| 3121 | tataaacata |

The Cx 26 cDNA coding reference sequence NG_008358.1 (SEQ ID NO.30) is shown below in Table 5B. An anti-connexin 26 polynucleotide may have the sequence of any polynucleotide sequence having 12 to 80 nucleotides of SEQ ID NO:30 (or any number of nucleotides between 12 and 80).

TABLE 5B

| | |
|---|---|
| 1 | atggattggg gcacgctgca gacgatcctg gggggtgtga acaaacactc caccagcatt |
| 61 | ggaaagatct ggctcaccgt cctcttcatt tttcgcatta tgatcctcgt tgtggctgca |
| 121 | aaggaggtgt ggggagatga gcaggccgac tttgtctgca acaccctgca gccaggctgc |
| 181 | aagaacgtgt gctacgatca ctacttcccc atctcccaca tccggctatg ggccctgcag |
| 241 | ctgatcttcg tgtccacgcc agcgctccta gtggccatgc acgtggccta ccggagacat |
| 301 | gagaagaaga ggaagttcat caagggggag ataaagagtg aatttaagga catcgaggag |
| 361 | atcaaaaccc agaaggtccg catcgaaggc tccctgtggt ggacctacac aagcagcatc |
| 421 | ttcttccggg tcatcttcga agccgccttc atgtacgtct tctatgtcat gtacgacggc |
| 481 | ttctccatgc agcggctggt gaagtgcaac gcctggcctt gtcccaacac tgtggactgc |
| 541 | tttgtgtccc ggcccacgga gaagactgtc ttcacagtgt tcatgattgc agtgtctgga |
| 601 | atttgcatcc tgctgaatgt cactgaattg tgttatttgc taattagata ttgttctggg |
| 661 | aagtcaaaaa agccagttta a |

The Cx 30 cDNA coding reference sequence NM_001110219.2 (SEQ. ID. NO:31) is shown below in Table 5C. An anti-connexin 30 polynucleotide may have the sequence of any polynucleotide sequence having between 12 to 80 nucleotides (or any number of nucleotides between 12 and 80) of SEQ ID NO:31.

TABLE 5C

| | |
|---|---|
| 1 | atggattggg ggacgctgca cactttcatc gggggtgtca acaaacactc caccagcatc |
| 61 | gggaaggtgt ggatcacagt catctttatt ttccgagtca tgatcctcgt ggtggctgcc |
| 121 | caggaagtgt ggggtgacga gcaagaggac ttcgtctgca acacactgca accgggatgc |
| 181 | aaaaatgtgt gctatgacca ctttttcccg gtgtcccaca tccggctgtg ggccctccag |
| 241 | ctgatcttcg tctccacccc agcgctgctg gtggccatgc atgtggccta ctacaggcac |
| 301 | gaaaccactc gcaagttcag gcgaggagag aagaggaatg atttcaaaga catagaggac |
| 361 | attaaaaagc agaaggttcg gatagagggg tcgctgtggt ggacgtacac cagcagcatc |
| 421 | tttttccgaa tcatctttga agcagccttt atgtatgtgt tttacttcct ttacaatggg |
| 481 | taccacctgc cctgggtgtt gaaatgtggg attgacccct gccccaacct tgttgactgc |
| 541 | tttatttcta ggccaacaga gaagaccgtg tttaccattt ttatgatttc tgcgtctgtg |
| 601 | atttgcatgc tgcttaacgt ggcagagttg tgctacctgc tgctgaaagt gtgttttagg |
| 661 | agatcaaaga gagcacagac gcaaaaaaat caccccaatc atgccctaaa ggagagtaag |
| 721 | cagaatgaaa tgaatgagct gatttcagat agtggtcaaa atgcaatcac aggtttccca |
| 781 | agctaa |

### Other Anti-Connexin Agents

As used herein, “gap junction phosphorylating agent” may include those agents or compounds capable of inducing phosphorylation on connexin amino acid residues in order to induce gap junction or hemichannel closure. Exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B, J. Biol. Chem., Vol. 276, Issue 11, 8544-8549, Mar. 16, 2001. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Still other anti-connexin agents include connexin carboxy-terminal polypeptides. See Gourdie et al., WO2006/069181.

In certain another aspect, gap junction modifying agent may include, for example, aliphatic alcohols; octanol; heptanol; anesthetics (e.g. halothane), ethrane, fluothane, propofol and thiopental; anandamide; arylaminobenzoate (FFA: flufenamic acid and similar derivatives that are lipophilic); carbenoxolone; Chalcone: (2′,5′-dihydroxychalcone); CHFs (Chlorohydroxyfuranones); CMCF (3-chloro-4-(chloromethyl)-5-hydroxy-2(5H)-furanone); dexamethasone; doxorubicin (and other anthraquinone derivatives); eicosanoid thromboxane A(2) (TXA(2)) mimetics; NO (nitric oxide); Fatty acids (e.g. arachidonic acid, oleic acid and lipoxygenase metabolites; Fenamates (flufenamic (FFA), niflumic (NFA) and meclofenamic acids (MFA)); Genistein; glycyrrhetinic acid (GA):18a-glycyrrhetinic acid and 18-beta-glycyrrhetinic acid, and derivatives thereof; lindane; lysophosphatidic acid; mefloquine; menadione; 2-Methyl-1,4-naphthoquinone, vitamin K(3); nafenopin; okadaic acid; oleamide; oleic acid; PH, gating by intracellular acidification; e.g., acidifying agents; polyunsaturated fatty acids; fatty acid GJIC inhibitors (e.g., oleic and arachidonic acids); quinidine; quinine; all trans-retinoic acid; and tamoxifen.

### Manufacture and Stability

The polynucleotides of this invention can be manufactured using solid-phase chemistries for synthesizing oligonucleotides. In one aspect, the formulations of this invention will comprise a salt of the polynucleotides of this invention, such as the sodium salt of the polynucleotides of this invention. In one embodiment the formulation may comprise the sodium salt of a polynucleotide having SEQ ID NO: 1, for example. In some embodiments, the polynucleotide having SEQ ID NO: 1 may be a modified oligodeoxynucleotide having SEQ ID NO:1.

In some embodiments, the formulations of this invention are substantially pure. By substantially pure is meant that the formulations comprise less than about 10%, 5%, or 1%, and preferably less than about 0.1%, of any nucleotide or non-nucleotide impurity. In some embodiments the total impurities, including metabolities of the connexin 43 modulating agent, will be not more than 15%. In some embodiments the total impurities, including metabolities of the connexin 43 modulating agent, will be not more than 12%. In some embodiments the total impurities, including metabolities of the connexin 43 modulating agent, will be not more than 11%. In other embodiments the total impurities, including metabolities of the connexin 43 modulating agent, will be not more than 10%.

In some embodiments, the purity of the formulations of this invention may be measured using a method selected from anion exchange HPLC (AEX-HPLC) or mass spectrometry. Mass spectrometry may include LC/MS, or LC/MS/MS. The assay may in some embodiments comprise both AEX-HPLC and LC/MS.

Sterile compositions comprising the connexin 43 modulating agents of this invention prepared using aseptic processing by dissolving the anti-connexin modulating agent in the formulation vehicle. In one embodiment, the formulation may also be sterilized by filtration. Excipients used in the manufacture of of the formulations of this invention are widely used in pharmaceutical products and released to pharmacopeial standards.

### Dosage Forms and Formulations and Administration

The connexin protein modulating agents of the invention, for example, connexin 43, 30 or 26 modulating agents may be administered to a subject in need of treatment, having a resistant wound, such as mVLU, or multiple DFU or pressure ulcers or other multiple non-healing, slow-healing, or chronic lesions. The anti-connexin 43 modulating agents may be used in the manufacture of a medicament to treat any of the conditions mentioned herein. Thus, in accordance with the invention, there are provided formulations by which connexin 43 can be modulated and/or cell-cell communication can be downregulated in a transient and site-specific manner.

The connexin protein modulating agent, or anti-connexin protein agent, may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of the polynucleotide (or other anti-connexin 43 agent) or dry mass of the preparation. In one embodiment, the anti-connexin agent is an anti-connexin 43, 30 or 26 peptide or anti-connexin 43, 30 or 26 peptidomimetic, e.g., an anti-connexin agent that can block or reduce hemichannel opening, is administered prior to the administration of an anti-connexin43 polynucleotide that blocks or reduce connexin expression or the formation of hemichannels or gap junctions, e.g., by downregulation of connexin protein expression.

The pharmaceutical formulations, or pharmaceutical compositions, combined preparations and medicaments of the invention may take any suitable form for topical administration. For example, the pharmaceutical formulations may take the form of solutions, suspensions, instillations, salves, creams, gels, foams, ointments, emulsions, lotions, paints, sustained release formulations, or powders, and typically contain about 0.1%-95% of active ingredient(s), preferably about 0.2%-70%. Other suitable formulations include pluronic gel-based formulations, carboxymethylcellulose (CMC)-based formulations, and hyroxypropylmethylcellulose (HPMC)-based formulations.

In some embodiments, the pharmaceutically acceptable carrier or vehicle is, or comprises, a gel. In one aspect the gel can be a reverse-thermosetting gel which is a liquid at low temperatures, for example at 2-8° C., and which undergoes a reversible liquid to gel transition at temperatures greater than approximately 15° C. Thus, in some embodiments the carrier may be a liquid at temperatures below approximately 15° C., but may form a gel at temperatures above approximately 15° C., such as room temperature or at body temperature. In some instances, the gel is a nonionic polyoxyethylene-polyoxypropylene copolymer gel. In some embodiments the gel is a pluronic gel. The pluronic gel may be, for example, poloxamer 407, also sometimes referred to as Pluronic F-127 (BASF). In some embodiments, the formulations of this invention may comprise from about 15 to about 30% (w/v) gel. In some embodiments, the formulations of this invention may comprise from about 20 to about 25% (w/v) gel. In some embodiments, the formulations of this invention may comprise about 22.6% (w/v) poloxamer 407 gel. In some embodiments, the gel may be a fluorinated methacrylamide chitosan hydrogel system. See, Wijekoon et al., Acta Biomater. 2013 March: 9(3):5653-64.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

An auxiliary agent such as casein, gelatin, albumin, glue, sodium alginate, carboxymethylcellulose, methylcellulose, hydroxyethylcellulose or polyvinyl alcohol may also be included in the formulation of the invention.

Other suitable formulations include pluronic gel-based formulations, carboxymethylcellulose (CMC)-based formulations, and hyroxypropylmethylcellulose (HPMC)-based formulations. The composition may be formulated for any desired form of delivery, including topical, instillation, parenteral, intramuscular, subcutaneous, or transdermal administration. Other useful formulations include slow or delayed release preparations.

Where the anti-connexin agent is a nucleic acid, such as a polynucleotide, uptake of nucleic acids by mammalian cells is enhanced by several known transfection techniques for example those including the use of transfection agents. Such techniques may be used with certain anti-connexin agents, including polynucleotides. The formulation which is administered may contain such transfection agents. Examples of these agents include cationic agents (for example calcium phosphate and DEAE-dextran) and lipofectants (for example Lipofectam™ and Transfectam™), and surfactants.

Where the anti-connexin agent comprises a polynucleotide, conveniently, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included. In certain non-limiting preferred embodiments, the transdermal penetration agent comprises an ethoxylated oil or fatty acid, fatty alcohol, or fatty amine therein having about 10 to 19 ethoxylations per molecule. Ethoxylated lipids suitable as a penetration enhancer include oils such as an ethoxylated vegetable, nut, synthetic or animal oil, suitably ethoxylated emu oil or ethoxylated macadamia nut oil. According to a non-limiting preferred aspect, suitable ethoxylated lipids that can be used in the formulations described herein can be a vegetable, nut, animal, or synthetic oil or fatty acid, fatty alcohol, or fatty amine therein having at least 10, 11, 12, 13, 14, 15, 16, 17, 18, 19, or more ethoxylations per molecule. Non-limiting preferred ethoxylated oils include macadamia nut oil, meadowfoam oil (limnanthes alba) castor oil, jojoba oil, corn oil, sunflower oil, sesame oil or emu oil. Optionally, other conventional agents used in pharmaceutical formulations such as an alcohol and/or water and/or an aqueous adjuvant can be mixed with the penetration enhancer to improve the solubility and/or transport of a particular gap junction modulation agent.

The effective dose for a given subject or condition can be determined by experimentation or other methods known in the art or later developed. For example, in order to formulate a range of dosage values for human subjects, cell culture assays and animal studies can be used, and doses providing superior results can be converted to doses for human or other mammalian subjects. The dosage of such compounds preferably lies within the dose that is therapeutically effective for at least 50% of the population, and that exhibits little or no toxicity at this level.

The effective dosage of each of the anti-connexin agents employed in the methods and compositions of the invention may vary depending on a number of factors including the particular anti-connexin agent or agents employed, whether used alone or in combination, the combination partner, the mode of administration, the frequency of administration, the severity fo the resistant lesion, the route of administration, the needs of a patient sub-population to be treated or the needs of the individual patient which can differ due to age, sex, body weight, relevant medical condition specific to the patient.

The dose at which an anti-connexin agent is administered to a patient will depend upon a variety of factors such as the age, weight and general condition of the patient, the condition that is being treated, and the particular anti-connexin agent that is being administered.

A suitable therapeutically effective dose of an anti-connexin agent may be at least about 1.0 mg/mL of the anti-connexin agent. In some embodiments, the suitable therapeutically effective dose of the anti-connexin agent may be from about 0.1 mg/mL to about 100 mg/mL. In some embodiments, the suitable therapeutically effective dose of an anti-connexin agent may be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 52.5, 55.0, 57.5, 60.0, 62.5, 65.0, 67.5, 70.0, 72.5, 75.0, 77.5, 80.0, 82.5, 85.0, 87.5, 90.0, 92.5, 95.0, 97.5, or about 100.0 mg/mL, or any range or subrange between any two of the recited doses, or any dose falling within the range of about 0.1 to about 100 mg/mL. In other embodiments, the connexin 43 modulating agent is present at a concentration ranging from about 0.5 to about 50 mg/mL. In other embodiments, the connexin 43 modulating agent is present at a concentration ranging from about 0.3 to about 30 mg/mL. In other embodiments, the connexin 43 modulating agent is present at a concentration ranging from about 0.1 or 1.0 to about 10 mg/mL. In other embodiments, the connexin 43 modulating agent is present at a concentration ranging from about 0.1 or 1.0 to about 0.3 or 3.0 mg/mL. In other embodiments, the connexin protein modulating agent, such as a connexin 43 modulatinge agent, a connexin 30 modulating agent and/or a connexin 26 modulating agent is present at a concentration of about 3.0 mg/mL. In any of these aspects the connexin 43, 30 or 26 modulating agent may be a connexin 43, 30 or 26 antisense oligonucleotide. When the connexin 43 modulating agent is a modified connexin 43 antisense oligonucleotide the above-noted dose concentrations may be increased by from about 2- to about 10-fold, for example. In any of these aspects, the carrier (vehicle) may be a thermoreversible gel. For example, the gel may be a poloxamer gel, for example, poloxamer 407, present in an amount ranging from about 15-25 or 30%, for example.

Alternatively, in the case of anti-connexin oligonucleotides or anti-connexin peptidomimetics, the dosage of each of the gap junction modulation agents in the compositions may be determined by reference to the composition's concentration relative to the size, length, depth, area or volume of the area to which it will be applied. For example, in certain topical applications, dosing of the pharmaceutical compositions may be calculated based on mass (e.g., grams) of or the concentration in a pharmaceutical composition (e.g., μg/ul) per length, depth, area, or volume of the area of application. Useful doses of polynucleotides range from about 3 to about 500 micrograms per square centimeter of wound size. Certain doses will be about 2 to about 10 micrograms per square centimeter of wound size. Doses may also be from about 3 to about 30 micrograms per square centimeter of wound size. Certain doses will be about 3-10, about 10-30, about 30-50, 50-75, 75-100, or about 30-100 micrograms per square centimeter of wound size. Other useful doses are greater than about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Other doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter, or any range or subrange between any two of the recited doses, or any dose falling within the range of about 3 to about 500 micrograms per square centimeter of wound size, or greater.

Useful doses ranges may also include from about 10 to 500 micrograms per square centimeter of wound size, including at least about 15 micrograms per square centimeter of wound size, at least about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Othe doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter. In other embodiments, the doses will be about 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 52.5, 55.0, 57.5, 60.0, 62.5, 65.0, 67.5, 70.0, 72.5, 75.0, 77.5, 80.0, 82.5, 85.0, 87.5, 90.0, 92.5, 95.0, 97.5, 100.0, 105, 110, 115, 120, 125, 130, 135, 140, 145, 150, 155, 160, 65, 170, 175, 180, 185, 190, 195, 200, 210, 220, 230, 250, 260, 270, 280, 290, 300, 310, 320, 330, 340, 350, 360, 370, 380, 390, 400, 410, 420, 430, 440, 450, 460, 470, 480, 490, or about 500 milligrams per square centimeter, or any range or subrange between any two of the recited doses, or any dose falling within the range of about 1.0 to about 500 milligrams per square centimeter.

In certain embodiments, the anti-connexin agent composition may be applied at about 0.01 micromolar (μM) or 0.05 τM to about 200 μM, or up to 300 μM or up to 1000 μM or up to 2000 μM or up to 3200 μM or more, for example up to about 10 mM, 20 mM, or 30 mM final concentration at the treatment site and/or adjacent to the treatment site, and any doses and dose ranges within these dose numbers. In one embodiment, the anti-connexin agent composition is applied at greater than about 1000 M. Preferably, the antisense polynucleotide composition is applied at about 1000 JAM to about 10 mM final concentration, more preferably, the anti-connexin agent composition is applied at about 3 mM to about 10 mM final concentration, and more preferably, the anti-connexin agent composition is applied at about 1-3 mM to about 5-10 mM final concentration.

Additionally, anti-connexin protein agents, such as anti-connexin 43, 30 or 26 agents, or other resistant wound healing agents may be present at about 8 μM to about 20 μM final concentration, and alternatively the anti-connexin agent composition is applied at about 10 μM to about 20 μM final concentration, or at about 10 to about 15 μM final concentration. In certain other embodiments, the anti-connexin agent is applied at about 10 μM final concentration. In yet another embodiment, the anti-connexin agent composition is applied at about 1-15 μM final concentration. In other embodiments, the anti-connexin agent is applied at about a 20 μM, 30 μM, 40 μM, 50 μM, 60 μM, 70 μM, 80 μM, 90 μM, 100 μM, or from about 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM, 2000 μM-3000 μM, 3000 μM-4000 μM, 4000 μM-5000 μM, 5000 μM-6000 μM, 6000 μM-7000 μM, 7000 μM-8000 μM, 8000 μM-9000 μM, 9000 μM-10,000 μM, 10,000 μM-11,000 μM, 11,000 μM-12,000 μM, 12,000 μM-13,000 μM, 13,000 μM-14,000 μM, 14,000 μM-15,000 μM, 15,000 μM-20,000 μM, 20,000 μM-30,000 μM, 30,000 μM-50,000 μM, or greater, or any range or subrange between any two of the recited doses, or any dose falling within the range of from about 20 μM to about 50,000 μM.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one anti-connexin agent is used, the dosage of each anti-connexin agent need not be in the same range as the other. For example, the dosage of one anti-connexin agent may be between about 0.01 mg to about 10 mg per kg body weight, and the dosage of another anti-connexin agent may be between about 0.1 mg to about 1 mg per kg body weight, 0.1 to about 10, 0.1 to about 20, 0.1 to about 30, 0.1 to about 40, or between about 0.1 to about 50 mg per kg body weight. The dosage may also be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 52.5, 55.0, 57.5, 60.0, 62.5, 65.0, 67.5, 70.0, 72.5, 75.0, 77.5, 80.0, 82.5, 85.0, 87.5, 90.0, 92.5, 95.0, 97.5, or about 100.0 mg per kg body weight, or any range or subrange between any two of the recited doses, or any dose falling within the range of from about 0.1 to about 100 mg per kg body weight.

Conveniently, the anti-connexin agent is administered in a sufficient amount to downregulate expression of a connexin protein, or modulate gap junction formation or connexon opening for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of the anti-connexin agents in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. μg/μl) per length, depth, area, or volume of the area of application. The volume of the wound may also be determined by imaging.

The doses of an anticonnexin protein modulating agent may be administered in single or divided applications. The doses may be administered once, or application may be repeated. Typically, application will be repeated weekly until wound healing is promoted, or a repeat application may be made in the event that wound healing slows or is stalled. Doses may be applied every 12 hours to 7 days apart, or more. For example, doses may be applied 12 hours, or 1, 2, 3, 4, 5, 6, or 7 days apart, or at any time interval falling between any two of these times, or between 12 hours and 7 days. In the case of a chronic wound, repeat applications may be made, for example, weekly, or bi-weekly, or monthly or in other frequency for example if and when wound healing slows or is stalled. The anti-connexin 43 agent, or connexin 43 modulating agent, may be administered for up to four, six, eight, ten, twelve, fourteen, sixteen, eighteen, twenty, twenty-two, twenty-four or twenty-six weeks. For some indications, such as certain ocular uses, more frequent dosing, up to hourly may employed.

In one aspect of the invention a an anti-connexin 26, 30 or 43 polynucleotide is administered in one composition and an anti-connexin 26, 30 or 43 polynucleotide is administered in a second composition. The first and second compositions may be administered simultaneously, separately or sequentially and in any order. For example, the first is administered before the second composition. In one embodiment the first composition is administered after the second composition. In one embodiment the first composition is administered before and after the second composition. In one embodiment the second composition is administered before and after the first composition. When not administered as a fixed combination, preferred methods include the sequential administration of one or more anti-connexin polynucleotides or one or more anti-connexin peptides or peptidomimetics, either or both of which are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered with about one hour of each other, with about one day to about one week of each other, or as otherwise deemed appropriate. As noted herein, the doses of an anti-connexin polynucleotide, peptide or peptidomimetic administered in combination, or other anti-connexin agents administered in combination with either or both, can be adjusted down from the doses administered when given alone.

In one embodiment, the combined use of one or more anti-connexin polynucleotides or one or more anti-connexin peptides or peptidomimetics reduces the effective dose of any such agent compared to the effective dose when said agent administered alone. In certain embodiments, the effective dose of the agent when used in combination is about 1/15 to about ½, about 1/10 to about ⅓, about ⅛ to about ⅙, about ⅕, about ¼, about ⅓ or about ½ the dose of the agent when used alone. In another preferred embodiment, the combined use of one or more anti-connexin polynucleotides and one or more anti-connexin peptides or peptidomimetics, or other anti-connexin agents in combination with either or both, reduces the frequency in which said agent is administered compared to the frequency when said agent is administered alone. Thus, these combinations allow the use of lower and/or fewer doses of each agent than previously required to achieve desired therapeutic goals.

Preferably one or more anti-connexin agents, such as anti-connexin 43 polynucleotides, are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-connexin polynucleotides and one or more anti-connexin peptides or peptidomimetics, or other anti-connexin agents to be administered in combination with either or both, is dispersed in a slow release solid matrix such as a matrix of alginate, collagen, or a synthetic bioabsorbable polymer. Preferably, the solid support composition is sterile or low bio-burden. In one embodiment, a wash solution comprising two or more anti-connexin agents can be used.

The delivery of of a formulation comprising one or more anti-connexin protein modulating agents (for example, anti-connexin 43, 30 or 26 modulating agents), such as polynucleotides or peptides or peptidomimetics, or other anti-connexin protein agents to be administered alone or in combination with either or both, over a period of time, in some instances for about 1-2 hours, about 2-4 hours, about 4-6 hours, about 6-8, or about 24 hours or longer, may be a particular advantage in more severe injuries or conditions. In some instances, cell loss may extend well beyond the site of a procedure to surrounding cells. Such loss may occur within 24 hours of the original procedure and is mediated by gap junction cell-cell communication, or hemichannel opening. Administration of anti-connexin agent(s), e.g., for downregulation of connexin expression, or blockade or inhibition of connexon opening or activity, therefore will modulate communication between the cells, or loss into the extracellular space in the case of connexon regulation, and minimize additional cell loss or injury or consequences of injury.

While the delivery period will be dependent upon both the site at which the downregulation is to be induced and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of one or more anti-connexin polynucleotides and/or one or more anti-connexin peptides or peptidomimetics, or other anti-connexin 43 agents or resistant wound healing agents, alone or in combination with either or both, in a formulation together with a pharmaceutically acceptable carrier or vehicle, particularly in the form of a formulation for continuous or slow-release administration.

The routes of administration and dosages described herein are intended only as a guide since a skilled physician will determine the optimum route of administration and dosage for any particular patient and condition.

Any of the methods of treating a subject having a resistant wound referenced or described herein may utilize the administration of any of the doses, dosage forms, formulations, and/or compositions herein described.

### Dressings and Matrices

In one aspect, one or more anti-connexin polynucleotides and/or one or more anti-connexin peptides or peptidomimetics are provided in the form of a dressing or matrix. In certain embodiments, the one or more agents of the invention are provided in the form of a liquid, semi solid or solid composition for application directly, or the composition is applied to the surface of, or incorporated into, a solid contacting layer such as a dressing gauze or matrix. The dressing composition may be provided for example, in the form of a fluid or a gel. One or more anti-connexin 43 polynucleotides and one or more anti-connexin 43 peptides or peptidomimetics may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In addition to the biological matrices previously mentioned, suitable dressings or matrices may include, for example, the following with one or more anti-connexin polynucleotides or one or more anti-connexin protein peptides or peptidomimetics (or other anti-connexin agents to be administered in combination with either or both):

1) Absorptives: suitable absorptives may include, for example, absorptive dressings, which can provide, for example, a semi-adherent quality or a non-adherent layer, combined with highly absorptive layers of fibers, such as for example, cellulose, cotton or rayon. Alternatively, absorptives may be used as a primary or secondary dressing.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source, e.g., pigs or cows. In certain embodiments, a gel or solution is applied to the treatment site and covered with a dressing for barrier protection. In another embodiment, a biological-based (e.g., cultured humans cells, pig intestinal mucosa or bladder tissue) or biosynthetic-based sheet is placed in situ which may act as membrane, remaining in place after a single application, or the may be biological dressings or biosynthetic dressings may be prepared in advance to include one or more, preferably two, anti-connexin agents.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with treatment site exudate to form a gel. In certain embodiments, collagen dressing may be used in combination with a secondary dressing.

6) Composites: suitable composite dressings may include, for example, dressings that combine physically distinct components into a single product to provide multiple functions, such as, for example, a bacterial barrier, absorption and adhesion. In certain embodiment, the composite dressings are comprised of, for example, multiple layers and incorporate a semi- or non-adherent pad. In certain embodiment, the composite may also include for example, an adhesive border of non-woven fabric tape or transparent film. In certain other embodiment, the composite dressing may function as for example, either a primary or a secondary dressing and in yet another embodiment, the dressing may be used in combination with topical pharmaceutical composition.

7) Contact Layers: suitable contact layer dressings may include, for example, thin, non-adherent sheets placed on an area to protect tissue from for example, direct contact with other agents or dressings applied to the treatment site. In certain embodiments, contact layers may be deployed to conform to the shape of the area of the treatment site and are porous to allow exudate to pass through for absorption by an overlying, secondary dressing. In yet another embodiment, the contact layer dressing may be used in combination with topical pharmaceutical composition.

8) Elastic Bandages: suitable elastic bandages may include, for example, dressings that stretch and conform to the body contours. In certain embodiment, the fabric composition may include for example, cotton, polyester, rayon or nylon. In certain other embodiments, the elastic bandage may for example, provide absorption as a second layer or dressing, to hold a cover in place, to apply pressure or to cushion a treatment site.

9) Foams: suitable foam dressings may include, for example, sheets and other shapes of foamed polymer solutions (including polyurethane) with small, open cells capable of holding fluids. Exemplary foams may be for example, impregnated or layered in combination with other materials. In certain embodiment, the absorption capability may be adjusted based on the thickness and composition of the foam. In certain other embodiments, the area in contact with the treatment site may be non-adhesive for easy removal. In yet another embodiment, the foam may be used in combination with an adhesive border and/or a transparent film coating that can serve as an anti-infective barrier.

10) Gauzes & Non-Woven dressings: suitable gauze dressings and woven dressings may include, for example, dry woven or non-woven sponges and wraps with varying degrees of absorbency. Exemplary fabric composition may include, for example, cotton, polyester or rayon. In certain embodiment, gauzes and non-woven dressing may be available sterile or non-sterile in bulk and with or without an adhesive border. Exemplary gauze dressings and woven dressings may be used for cleansing, packing and covering a variety of treatment sites.

11) Hydrocolloids: suitable hydrocolloid dressings may include, for example, wafers, powders or pastes composed of gelatin, pectin or carboxymethylcellulose. In certain embodiment, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable amorphous hydrogel dressings may include, for example, formulations of water, polymers and other ingredients with no shape, designed to donate moisture and to maintain a moist healing environments and or to rehydrate the treatment site. In certain embodiment, hydrogels may be used in combination with a secondary dressing cover.

13) Hydrogels: Impregnated Dressings: suitable impregnated hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry treatment site and to maintain a moist healing environment.

14) Hydrogel Sheets: suitable hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the treatment site or treated for easy removal.

15) Impregnated Dressings: suitable impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform and scarlet red as well as the compounds described herein.

16) Silicone Gel Sheets: suitable silicone gel sheet dressings may include, for example, soft covers composed of cross-linked polymers reinforced with or bonded to mesh or fabric.

17) Solutions: suitable liquid dressings may include, for example, mixtures of multiprotein material and other elements found in the extracellular matrix. In certain embodiment, exemplary solutions may be applied to the treatment site after debridement and cleansing and then covered with an absorbent dressing or a nonadherent pad.

18) Transparent Films: suitable transparent film dressings may include polymer membranes of varying thickness coated on one side with an adhesive. In certain embodiments, transparent films are impermeable to liquid, water and bacteria but permeable to moisture vapor and atmospheric gases. In certain embodiments, the transparency allows visualization of the treatment site.

19) Fillers: suitable filler dressings may include, for example, beads, creams, foams, gels, ointments, pads, pastes, pillows, powders, strands or other formulations. In certain embodiment, fillers are non-adherent and may include a time-released antimicrobial. Exemplary fillers may be useful to maintain a moist environment, manage exudate, and for treatment of for example, partial- and full-thickness wounds, infected wounds, draining wounds and deep wounds that require packing.

### Kits, Medicaments and Articles of Manufacture

Optionally, one or more anti-connexin protein polynucleotides and/or one or more anti-connexin protein peptides or peptidomimetics and/or other anti-connexin agents such as a gap junction or hemichannel phosphorylation agent or connexin carboxy-terminal polypeptide, alone or in combinations of any of the anti-connexin protein modulating agents, or other resistant wound healing agents, may also be used in the manufacture of the medicament, or in a kit. Suitable anti-connexin protein modulating agents, polynucleotides or peptides may be anti-connexin 43, 30 or 26 modulating agents, polynucleotides or peptides.

In one aspect, the invention provides an article of manufacture or kit comprising one or more compositions or formulations described. For example, the kit may include a pharmaceutical formulation comprising an effective amount of one or more anti-connexin 43 polynucleotides and/or one or more anti-connexin 43 peptides or peptidomimetics and/or other anti-connexin agents, such as a gap junction or hemichannel phosphorylation agent or connexin carboxy-terminal polypeptide, alone or in combinations of any of the anti-connexin 43 modulating agents, or other resistant wound healing agents,

Articles of manufacturer are also provided, comprising a vessel containing a composition or formulation of the invention as described herein and instructions for use for the treatment of a subject. For example, in another aspect, the invention includes an article of manufacture comprising a vessel containing a therapeutically effective amount of one or more anti-connexin protein polynucleotides and/or one or more anti-connexin protein peptides or peptidomimetics and/or other anti-connexin agents, such as a gap junction or hemichannel phosphorylation agent or connexin carboxy-terminal polypeptide, alone or in combinations of any of the anti-connexin protein modulating agents, or other resistant wound healing agents, together with instructions for use, including use for the treatment of a subject. Suitable anti-connexin protein modulating agents, polynucleotides or peptides may be anti-connexin 43, 30 or 26 modulating agents, polynucleotides or peptides.

In some aspects the article of manufacture may comprise a matrix that comprises one or more anti-connexin protein peptides or peptidomimetics or other anti-connexin agents, such as a gap junction or hemichannel phosphorylation agent or connexin carboxy-terminal polypeptide, alone or in combinations of any of the anti-connexin 43 modulating agents, or other resistant wound healing agents. Suitable anti-connexin protein modulating agents, polynucleotides or peptides may be anti-connexin 43, 30 or 26 modulating agents, polynucleotides or peptides.

### Treatment

The compositions and formulations of the invention comprising one or more connexin protein modulating agents may be used for treating resistant lesions, such as mVLUs or mDFUs in responder subjects. The compositions and formulations of the invention may also be used in conjunction or combination with a second composition for promoting and/or improving the healing of resistant lesions.

As disclosed herein suitable anti-connexin protein polynucleotides, peptides or peptidomimetics or modulating agents for use in the methods of treatment of this invention may include, for example, anti-connexin 43, 30 or 26 polynucleotides or peptides or peptidomimetics.

In one aspect the invention is directed to a method of promoting or improving resistant lesion healing in a subject, comprising administration a therapeutically effective amount of one or more anti-connexin protein modulating agents, which may include anti-connexin protein polynucleotides and one or more anti-connexin protein peptides or peptidomimetics or, optionally, one or more anti-connexin protein polynucleotides and/or one or more anti-connexin 43 peptides or peptidomimetics other anti-connexin agents, such as a gap junction or hemichannel phosphorylation agent or connexin carboxy-terminal polypeptide, or other resistant wound healing agent. In certain embodiments, the administration of one or more anti-connexin protein polynucleotides and one or more anti-connexin protein peptides or peptidomimetics, or, optionally, one or more anti-connexin polynucleotides and/or one or more anti-connexin peptides or peptidomimetics other anti-connexin agents, or other resistant wound healing agent, is effective to improve healing of the resistant lesion, for example, to facilitate epithelial growth and surface recovery. In certain embodiments, the administration of one or more anti-connexin protein polynucleotides and one or more anti-connexin protein peptides or peptidomimetics, or, optionally, one or more anti-connexin polynucleotides and/or one or more anti-connexin peptides or peptidomimetics other anti-connexin agents, or other resistant wound healing agent, is effective to promote complete wound closure, or to increase the rate of persitent wound closure. According to another aspect of the present invention, re-epithlialization and/or formation of granulation tissue is promoted. Methods of promoting re-epithelialization of resistant skin lesions comprise administering to a subject having a resistant skin lesion, including, for example, mVLUs, in an amount effective to promote re-epithelialization. Analogous methods can be used to regulate epithelial basal cell division and growth. In certain embodiments, the administration of the anti-connexin protein modulating agent is effective to promote cell migration to accelerate closure and healing, to facilitate epithelial growth, or any combination thereof. Subjects which may be treated include subjects with mVLU, having one or more of the other indicators described herein, for example, age over 50-52 or BMI less than 40-42. Suitable anti-connexin protein modulating agents, polynucleotides or peptides may be anti-connexin 43, 30 or 26 modulating agents, polynucleotides or peptides.

In one aspect the invention is directed to a method of promoting or improving resistant lesion healing in a subject, comprising administration of one or more anti-connexin protein polynucleotides and one or more anti-connexin protein peptides or peptidomimetics, or, optionally, one or more anti-connexin protein polynucleotides and/or one or more anti-connexin protein peptides or peptidomimetics other anti-connexin agents, or resistant lesion healing agents, in an amount effective to regulate epithelial basal cell division and growth. In one embodiment, the anti-connexin agent is a connexin antisense polynucleotide effective to regulate epithelial basal cell division and growth. In one embodiment, a second connexin antisense polynucleotide is a connexin 26 or connexin 30 antisense polynucleotide, peptide or peptidomimetic, a connexin 43 antisense polynucleotide, peptide, or peptidomimetic or a mixture thereof. Subjects which may be treated include subjects with mVLU, having one or more of the other indicators described herein, for example, age over 50-52 or BMI less than 40-42.

In one aspect the invention is directed to a method of promoting or improving resistant wound healing, comprising administration of one or more anti-connexin protein peptides or peptidomimetics, or, optionally, one or more anti-connexin protein polynucleotides and/or one or more anti-connexin protein peptides or peptidomimetics other anti-connexin agents, or resistant wound healing agents, in an amount effective to regulate outer layer keratin secretion. In one embodiment, the anti-connexin agent is a connexin antisense polynucleotide effective to regulate outer layer keratin secretion. In one embodiment, the connexin antisense polynucleotide is a connexin protein antisense polynucleotide, peptide or peptidomimetic, a connexin 43, connexin 26 or connexin 30 antisense polynucleotide, peptide or peptidomimetic or a mixture thereof. Subjects which may be treated include subjects with mVLU, having one or more of the other indicators described herein, for example, age over 50-52 or BMI less than 40-42.

In yet a further aspect, the invention provides a method of decreasing scar formation and/or improving scar appearance in a patient who has suffered a resistant wound.

In one aspect the invention is directed to sustained administration of one or more anti-connexin protein polynucleotides and one or more anti-connexin protein peptides or peptidomimetics, or, optionally, one or more anti-connexin protein polynucleotides and/or one or more anti-connexin protein peptides or peptidomimetics other anti-connexin agents, or resistant wound healing agents. In one embodiment, the anti-connexin agents are administered for at least at least about 0.5 hours, about 1-24 hours, at least about 2, hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours or at least about 24 hours. In one embodiment, connexin expression is downregulated over a sustained period of time. In another embodiment, connexin hemichannels are blocked or closed, in whole or in part, over a preferred period of time. Preferably connexin protein expression is downregulated and connexin hemichannel opening is blocked or inhibited, in whole or in part, for a sustained period of time. Conveniently, connexin protein expression is downregulated or hemichannels blocked or inhibited for at least about 1, 2, 4, 6, 8, 10, 12, or 24 hours. According to one embodiment, the wound is a resistant lesion. Suitable subjects include a diabetic subject. Other subjects include, for example, those with peripheral edema, vasculitis, or cardiovascular disease. Suitable anti-connexin protein polynucleotides, peptides or peptidomimetics may be anti-connexin 43, 30 or 26 polynucleotides or peptides or peptidomimetics.

In one aspect, the present invention provides a method of treating a subject having a resistant wound which comprises sustained administration of an effective amount of one or more anti-connexin protein peptides or peptidomimetics, or, optionally, one or more anti-connexin protein polynucleotides and/or one or more anti-connexin protein peptides or peptidomimetics other anti-connexin agents, or resistant wound healing agents, to the wound.

According to another further aspect, the present invention provides a method of promoting or improving resistant wound healing in a subject having a wound which comprises sustained administration of one or more anti-connexin protein peptides or peptidomimetics, or, optionally, one or more anti-connexin protein polynucleotides and/or one or more anti-connexin protein peptides or peptidomimetics other anti-connexin agents, or resistant wound healing agents, to a wound area in an amount effective to increase re-epithlialization rates in the wound area.

In one embodiment, the composition or compositions are administered in a sustained release formulation. In another embodiment, the composition or compositions are administered for a sustained period of time. Conveniently, the composition is effective to decrease connexin protein alone, or in combination with reducing connexin 31.1 levels or activity (e.g., hemichannel or gap junction activity) for at least about 24 hours.

Subjects which may be treated include subjects with mVLU, having one or more of the other indicators described herein, for example, age over 50-52 or BMI less than 40-42. Subjects which may be treated include diabetic subjects.

In one aspect the invention is directed to a method for treatment or prophylaxis of a resistant lesion comprising administering to a subject in need thereof an effective amount of an anti-connexin agent administered to said resistant wound or a tissue associated with said resistant wound in combination with another anti-connexin agent. In another embodiment, the resistant wound is a resistant chronic skin lesion and a composition of the present invention is administered to the skin or a tissue associated with the skin of said subject for an effective period of time. Resistant lesions or wounds include multiple VLUs, multiple diabetic foot ulcers (DFUs), multiple pressure ulcers, wounds whose surface areas change relatively little during a screening period with compression bandaging or other standard-of-care therapy (e.g., off-loading) and with relatively few signs of healing during a screening period with compression bandaging therapy. In some aspects resistant lesions are characterized by less granulation and epithelialization during the screening period, or at the time of treatment with the connexin 43 modulating agent. The screening period may be from about 10 days to about 1-4 weeks, for example, and is typically 2 weeks and sometimes 4 weeks.

In some embodiments, the surface of the lesion may be freed of slough, exudate and devitalized tissue, preferably without excision of skin edges or enlargement of the lesion. In some embodiments, the pharmaceutical formulations of this invention comprising one or more connexin protein modulating agens may be applied topically around the inside edge of the ulcer to be treated and then applied to the remainder of the wound bed.

When not administered as a fixed combination, preferred methods include the sequential administration of one or more anti-connexin protein polynucleotides and one or more anti-connexin protein peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, or another resistant wound healing agent. Preferably, the agents are administered sequentially within at least about one-half hour of each other. The agents may also be administered with about one hour of each other, with about one day to about one week of each other, or as otherwise deemed appropriate. Preferably, an anti-connexin protein peptide or anti-connexin protein peptidomimetic, e.g., an anti-connexin agent that can block or reduce hemichannel opening, is administered prior to the administration of an anti-connexin agent that blocks or reduce connexin expression or the formation of hemichannels or gap junctions, e.g., by downregulation of connexin protein expression.

In another embodiment for treatment of wounds, including resistant wounds, either or both of the one or more anti-connexin protein polynucleotides and one or more anti-connexin protein peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, or other resistant wound healing agents, are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

In one embodiment the method for treatment or prophylaxis of a resistant wound comprises sustained administration of one or more anti-connexin protein polynucleotides and one or more anti-connexin protein peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, or other resistant wound healing agent. In one embodiment, the composition or compositions are administered in a sustained release formulation. In another embodiment, the composition or compositions are administered for a sustained period of time. Conveniently, the composition is effective to decrease connexin protein levels, or block or reduce connexin protein hemichannel opening, for at least about 1-2 hours, about 2-4 hours, about 4-6 hours, about 4-8 hours, about 12 hours, about 18 hours, or about 24 hours. Subjects which may be treated include diabetic subjects, and patients with other ulcers, including venous ulcers and others described herein and known in the art.

The following examples which will be understood to be provided by way of illustration only and not to constitute a limitation on the scope of the invention.

### EXAMPLES

### Example 1

### Lack of Toxicity

As discussed herein, the unmodified 30-mer anti-connexin deoxyoligonucleotide having SEQ ID NO:1 (“the Polynucleotide”) has been shown to have surprising utility in treating responder subjects with mVLUs and other indicators of likelihood to respond to treatment with an anti-connexin 43 modulating agent.

Moreover, use of the compositions of this invention, comprising synthetic, unmodified deoxyoligonucleotides with unmodified backbones resulted in low toxicity with no systemic exposure, and, importantly with respect to safety, undetectable or exceedingly low pK even when very large clinical-multiple doses of an unmodified anti-connexin deoxyoligonucleotide having SEQ. ID. NO: 1 were repeatedly administered to open wounds in the skin of test animals.

The low toxicity is due in part to the high specificity of the Polynucleotide. Human DNA sequence database searches were performed to evaluate the extent to which a polynucleotide having SEQ ID NO: 1 may have homology with sequences in the known array of human genes and to assess whether unwanted inhibitory activity could be exerted against expression of human gene products other than the target gene and thereby induce “off-target” effects. The human genome database searches for homologies within the genome, homologies with known and predicted transcripts, and homologies with potential internal mismatch sequences, showed that the 30-nucleotide oligonucleotide having SEQ ID NO:1 is highly specific for the intended CX43 target with no likely off-target effects.

In addition, in contrast with chemically modified oligonucleotides, which have been found to cause complement activation and inhibition of the extrinsic coagulation pathway, unmodified oligonucleotides of this invention have not shown such effects. Furthermore, the Polynucleotide displayed no evidence for genetic toxicity based on the results of the complete battery of three genetic toxicity studies (i.e., a bacterial mutagenicity assay, an in vitro chromosomal aberrations test and an in vivo micronucleus study in mice). Moreover, because the Polynucleotide is a chemically unmodified polynucleotide, it is degraded via naturally occurring processes such as depurination followed by backbone cleavage. Furthermore, oligonucleotides with an unmodified backbone like the Polynucleotide do not bind to plasma proteins (e.g., Brown D A, et al. Effect of phosphorothioate modification of oligodeoxynucleotides on specific protein binding. J Biol. Chem. 1994; 269:26801-26805), as do the phosphorothioate oligonucleotides, and hence, would not be expected to displace other drugs that bind to albumin or otherwise alter the balance of free vs. plasma protein-bound drug. Moreover, a single site modification in the 30-mer Polynucleotide will not dramatically alter the binding energy of the other 29 base pairs in the sequence and thus the related impurities will not be expected to alter the efficacy or specificity of the drug in a biological system.

The Polynucleotide has also been shown to have a short half-life in cells (˜20 minutes) and a very short half-life in the circulation (≦several minutes) due to rapid metabolism by endogenous nucleases and extremely rapid glomerular filtration. It has been shown that systemic exposure to the Polynucleotide is exceedingly low, even when very large clinical-multiple doses of a formulation comprising a poloxamer gel and anti-connexin deoxyoligonucleotide having SEQ. ID. NO:1 (Polynucleotide Formulation) are repeatedly administered to open wounds in the skin of test animals. Pharmacokinetic studies undertaken have also shown that the polynucleotide having SEQ ID NO: 1 is undetectable in the plasma from patients that have been treated topically with the Polynucleotide Formulation, despite the use of a highly sensitive hybridization type bioanalytical assay. Hence, systemic exposure of topically applied Polynucleotide Formulation across the range of clinical doses used is negligible.

For example, the Polynucleotide Formulation was well tolerated and revealed no toxicity when administered weekly to wound sites for 3 months in rats and rabbits at large clinical-multiple doses. Toxicokinetic and tissue distribution analyses showed that while there was substantial exposure of the wound site tissues to active oligonucleotide ingredient having SEQ ID NO: 1 over the course of the study, systemic exposure was negligible.

In both rat and rabbit dermal studies, rabbit subjects received weekly topical application of the Polynucleotide Formulation to excisional wound sites at doses of 0, 120, 1200 or 9320 ug/dose while rats received doses of 0, 30, 300 or 2330 ug/dose. The 13-week dermal toxicity studies in rats and rabbits, from which plasma and tissue samples were collected for Polynucleotide bioanalysis, were conducted with weekly topical application of the Polynucleotide Formulation to excisional wound sites for 2 weeks or 13 weeks (animals in the 13 week group were repeatedly wounded every 3 weeks), followed by a 2- or 4-week recovery-period. Analysis of plasma concentrations at several post-dosing time points revealed low systemic exposure even at the highest dose levels. Mean concentrations of the Polynucleotide were generally less than 100 ng/mL in plasma samples of animals treated with the highest dose levels (i.e., 9320 and 2330 μg/dose, for rabbits and rats, respectively, or approximately 3 and 4-6 mg/kg, respectively, based average body weights). For the majority of the samples collected from the low- and mid-dose rats and rabbits, no quantifiable levels of the Polynucleotide were present (levels were below LOQ of 1 ng/mL). Rapid metabolism and clearance of the Polynucleotide were observed in both species as evidenced by the rapid appearance of metabolites described as shortmers which are primarily N-1 and N-2 oligonucleotides with subsequent metabolism to shorter oligonucleotide structures. The Polynucleotide and shortmers were absent at the 3-hour post-dose time point, which is consistent with the expected rapid in vivo metabolism and clearance of an unmodified (natural backbone) oligonucleotide. Based on the nature of the bioanalytical assay employed (a hybridization assay with electrophorectic resolution), the expected exonuclease-mediated metabolism to chain-shortened metabolites was documented.

The results of the analysis of tissues collected at four time points in the rabbit study showed that the levels of the Polynucleotide and metabolites were very low or not quantifiable in the two internal organs analyzed (liver and kidney). The kidney and liver were chosen to assess systemic absorption because these are the known major organs of uptake of oligonucleotides with systemic administration. The absence of the Polynucleotide or shortmer metabolites in most of these samples is consistent with the plasma level data and indicates minimal systemic absorption of the Polynucleotide following topical application of the Polynucleotide Formulation to wound sites. In contrast, there was high and resistant exposure of the wound site to the Polynucleotide and metabolites following topical application of the Polynucleotide Formulation, with dose related mean levels of the Polynucleotide and metabolites present at wound sites, and decreasing amounts present one to four weeks after administration of the last dose. Although steady clearance of the Polynucleotide from the wound site skin was evident, as well as ongoing metabolism, it was found that although the Polynucleotide is unmodified, there was ample exposure of the wound site skin to the Polynucleotide throughout the study with the weekly dosing schedule that was utilized which was analogous to the clinical dosing schedule for all human VLU studies.

For both species, the levels of intact Polynucleotide or presumed metabolites in plasma and systemic tissues were much lower (generally not detectable) when samples were collected on days on which the dose was applied to a wound site that was largely healed (i.e., 15-16 days after wounding), as compared to the days when doses were applied to fresh wounds. Thus, the extent of systemic exposure to the Polynucleotide and metabolites was virtually negligible when the Polynucleotide Formulation was applied to a largely healed wound site, indicating that the Polynucleotide has little or no potential to cross an intact skin barrier. Overall, the plasma concentration data indicate that systemic absorption of the Polynucleotide when applied topically to wounds is very low, particularly when applied to partially healed wounds, and that nuclease-mediated metabolism occurs very quickly.

Further evidence of the minimal systemic exposure to the Polynucleotide was provided by the toxicokinetic data from the safety pharmacology study in cynomolgus monkeys in which large intravenous doses of the Polynucleotide were administered by bolus injection. In addition, the potential for large intravenous doses to elicit class effects that have been observed with chemically modified oligonucleotides (unlike the Polynucleotide), i.e., complement activation and inhibition of the extrinsic coagulation pathway, was assessed in this study. The Polynucleotide was admistered as single escalating doses of 10 and 50 mg/kg by intravenous bolus injection, and the animals were evaluated for changes in cardiovascular parameters by radiotelemetry (blood pressure, heart rate, and electrocardiographic activity (both qualitative and quantitative evaluation of ECG intervals), as well as respiratory function and neurologic function. Plasma samples for bioanalysis were collected at a few time points following intravenous injection. Doses of the Polynucleotide up to 50 mg/kg, administered intravenously, were associated with maximal plasma concentrations (at five minutes after dosing) of parent and proximal metabolites that ranged from approximately 230,000 to 460,000 ng/mL; however, the plasma concentrations had fallen to just above the lower limit of quantification (0.9 ng/mL) at the 2-hour post-dose collection time point. These data provide additional evidence for the rapid metabolism and clearance of the Polynucleotide.

Furthermore, the absence of any adverse effects in this study demonstrated safety under conditions when systemic exposure is several orders of magnitude greater than that detectable following administration by the intended clinical route, i.e., dermal application. Thus, the compositions and formulations of this invention have surprising low toxicity.

In addition, while chemically modified oligonucleotides have been found to cause complement activation and inhibition of the extrinsic coagulation pathway, these so-called “class effects” of chemically-modified oligonucleotides were not found with the Polynucleotide.

Human clinical PK studies also demonstrated that following administration of the anti-connexin deoxyoligonucleotide having SEQ ID NO: 1, the deoxyoligonucleotide was undetectable in the plasma of patients who had been treated topically for VLU with dose concentrations up to and including 3.0 mg/mL, using a bioanalytical method having a lower limit of quantification (LLOQ) of 1.0 ng/mL, indicating that there was negligible systemic exposure. Specifically, clinical use of the Polynucleotide Formulation has been associated with no measurable systemic absorption, which is expected for unmodified oligonucleotides that are characterized by poor metabolic stability in blood and are very rapidly eliminated via glomerular filtration and by nuclease-mediated metabolism. Thus, what little drug may enter the systemic circulation is rapidly metabolized to smaller natural-structure oligonucleotides or monomers and rapidly cleared by renal filtration. Also, the Polynucleotide has been shown to have high specificity to connexin 43 based on human genome database searches. These characteristics contribute to the overall favorable safety profile in which over 200 patients have been exposed to the Polynucleotide Formulation at 3.0 mg/mL or higher.

In summary, dermal administration of the Polynucleotide Formulation results in drug deposition in skin samples from wound sites. Following dosing of the wound site, the Polynucleotide is taken up into the skin and local tissues and persists at appreciable levels over a one-week period, which is the intended clinical dosing interval. However, there is no appreciable accumulation at the treatment site. Systemic exposure with this route of administration is negligible, mainly owing to a DNA structure that undergoes very rapid metabolism and elimination when taken up into the systemic circulation such that there is virtually no deposition in expected systemic tissues seen with other oligonucleotides, such as the kidney and liver.

### Example 2

### Analysis of Connexin 43 Levels in Single and Multiple VLUs

A study was undertaken of Cx43 protein expression in patients who were reported to have multiple (n=10; mVLU) or single (n=8; sVLU) venous leg ulcers. These patients were being treated for a clinical diagnosis of non-infected VLU of at least four weeks duration. They underwent a 4 mm punch chronic wound edge biopsy, and a matching punch of non-wounded arm skin. Cx43 protein expression was assessed by immunohistochemistry at three sites across the wound biopsy. The biopsy measurement sites were: (i) at the chronic wound edge side of the biopsy (WE), (ii) 1 mm away from the chronic WE side, and (iii) the opposite side to the chronic WE (far edge). Normal unwounded skin was assessed in one central site in the biopsy. Cx43 expression in the wound was normalized to the patient's non-wounded basal Cx43 expression (i.e., reported as the ratio of chronic wound edge skin Cx43 to the matched patient Cx43 expression in unwounded skin).

Biopsies:

Wound biopsies were taken during an outpatient clinic visit of a single treating clinician. A 4 mm full thickness skin punch biopsy taken under local anaesthetic from the visible wound edge (WE). The side of the biopsy away from the open wound-bed was marked with ink to help keep the sample orientated throughout subsequent histological processing. Each patient also provided a matched 4 mm punch biopsy of their normal arm skin, thus providing matched unwounded baseline Cx43 skin expression levels. At the time of collection samples were immediately transferred to 4% paraformaldehyde for 24 hours, and then 20% sucrose in Phosphate-buffered saline (“PBS”). Tissue blocks were then embedded in optimal cutting temperature medium (OCT) and stored at −80° C.

Immunohistochemistry:

Tissue was sectioned, stained and imaged by confocal microscopy using identical parameters per patient to permit quantification. Standard approach using primary antibody of Cx43 1:4000 (Sigma—Poole, UK—C6219). The Secondary antibody was Alexa Fluor® 488 goat anti-rabbit 1:400) (Invitrogen—Paisley, UK). Nuclei were stained using HOECHST (Sigma—Poole, UK—B-2883 and B-2261 1:50,000 in PBS).

Confocal Microscopy:

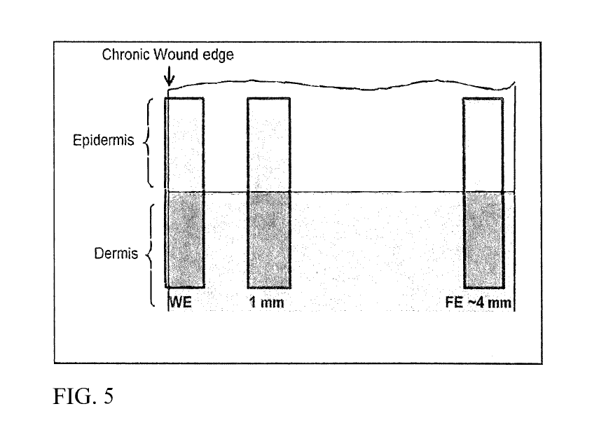

An Olympus FV-1000 inverted confocal microscope was used to take 40× images of the arm skin and wound. The 4 mm unwounded arm biopsies were assessed at one central site in the biopsy. The 4 mm wound biopsies were examined across their diameter at three locations: at the wound edge “WE”, “1 mm from the WE”, and at the far edge “FE” of the 4 mm biopsy (i.e., directly opposite the wound WE). FIG. 5 shows where the assessments were taken across the 4 mm biopsy.

Image Quantification and Statistical Analysis:

Cx43 quantification was carried out using ImageJ. Thresholds were kept constant between all images. Three different Cx43 expression measurement locations across the biopsy (i.e., “WE”, “1 mm”, “FE”) were independently compared between the multiple and single ulcer groups at those same three locations. Data were tested for normality and if necessary underwent a log-transformation before proceeding with a simple statistical approach comprising independent t-tests. Each of the three wound locations was treated as independent and no multiple comparison correction was applied. The mean raw data and SEM are shown on the graphs. Note: not all biopsies were suitable for assessment of Cx43 at all three locations in the biopsy, so in some cases a full dataset was not available. Final group sizes were 5-8 wounds from single wound patients and 8-10 wounds multiple wound patients.

TABLE 6
Patient Demographic Summary

| | N | | Age (yrs) | Wound Size (cm²) | Wound Duration (mths) |
|---|---|---|---|---|---|
| | (max) | M:F | (Median: Range) | (Median: Range) | (Median: Range) |
| Multiple | 10 | 5M:5F | 52 | 9.6 | 6 |
| wounds | | | (31-70) | (2.0-36.4) | (1-36) |
| Single | 8 | 6M:2F | 59 | 7.6 | 6 |
| wound | | | (45-79) | (2.4-113.1) | (1.5-108) |
| P value | | | 0.1 | 0.48 | 0.36 |

As shown in Table 6, the demographics of the two small study groups were not significantly different.

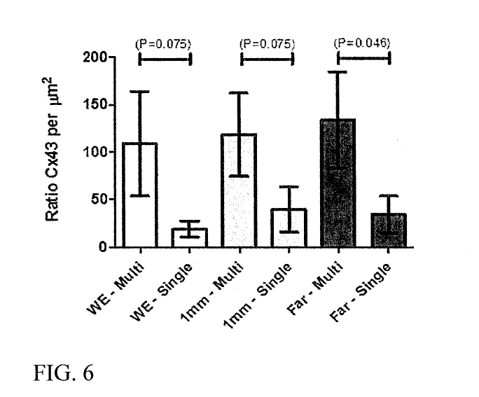

FIG. 6 shows Cx43 in dermis normalised to patient baseline expression (Ratio Dataset). There was a discernible pattern evident with a higher Cx43 expression ratio being present in the dermis of the “multiple wounds” compared to the “single wounds”. This was present across the whole biopsy. The statistical analysis (t-test on normalised data) between the groups supported this trend at the WE (p=0.07) and 1 mm (p=0.07) sites, and on the “Far” side of the biopsy it was significant (p=0.046).

There is usually low Cx43 background expression in the normal skin dermis compared to the epidermis. Cx43 upregulation in the dermis may be due to several factors such as increased underlying inflammatory cell invasion, new blood vessel formation, more myofibroblast differentiation, and a greater global stimulus on resident cells to express Cx43 due to the effects of surrounding tissue ischemia and hypoxia. These are all potential theoretical sources for the dermal Cx43 upregulation.

The existence of greater dermal Cx43 upregulation in patients with multiple wounds is consistent with “multiple” wounds having more underlying tissue damage, worse circulation, and/or more inflammation than “single” wounds. Indeed the formation of a “multiple” wound phenotype may even point to a different underlying biology with more “field changes” in the surrounding skin of the wounds than in “single phenotype” VLUs. In support of this concept, there is a literature on the differences between multiple or single wounds, showing that multiple wounds are associated with slower healing indices (Margolis D J, et al. The accuracy of venous leg ulcer prognostic models in a wound care system. Wound Repair and Regeneration. 2004; 12(2): 163-8) and considered a sign of worse underlying venous disease (Rutherford R B, et al. Venous severity scoring: An adjunct to venous outcome assessment. Journal of Vascular Surgery. 2000; 31(6): 1307-12). The increased dermal Cx43 expression in multiple leg ulcers may result from a greater inflammatory response and possibly impaired skin perfusion. Thus, the anti-inflammatory (Mori R, et al. Acute downregulation of connexin43 at wound sites leads to a reduced inflammatory response, enhanced keratinocyte proliferation and wound fibroblast migration. Journal of Cell Science. 2006; 119(Pt 24): 5193-203; Cronin M, et al. Blocking connexin43 expression reduces inflammation and improves functional recovery after spinal cord injury. Molecular and Cellular Neurosciences. 2008; 39(2): 152-60; Qiu C, et al. Targeting connexin43 expression accelerates the rate of wound repair. Current Biology. 2003; 13(19): 1697-703; Coutinho P, et al. Limiting burn extension by transient inhibition of Connexin43 expression at the site of injury. British journal of plastic surgery. 2005; 58(5): 658-67; Gilmartin D J, et al. Integration of scaffolds into full-thickness skin wounds: the connexin response. Advanced Healthcare Materials. 2013; 2(8): 1151-60), anti-vessel leak (Cronin M, et al. Blocking connexin43 expression reduces inflammation and improves functional recovery after spinal cord injury. Molecular and Cellular Neurosciences. 2008; 39(2): 152-60) and vascular regeneration (Ormonde S, et al. Regulation of connexin43 gap junction protein triggers vascular recovery and healing in human ocular resistant epithelial defect wounds. The Journal ofMembrane Biology. 2012; 245(7): 381-8) activities by connexin 43 modulation (see Example 3 below) i.e., the specific activities not known to be caused directly by a vehicle plus compression or compression bandaging alone, are more evident in this treatment group. Multiple wounds likely represent the end result of more extensive underlying tissue damage and pathology compared to single wounds, and this severity can manifest as higher Cx43 expression, the target of connexin modulators such as, for example, a Cx43 antisense oligonucleotide. It was surprisingly determined that multiple VLU representing more severe, and generally harder to heal lesions, may respond to a wider range of modulated Cx43 activities, e.g., anti-inflammatory activity, vascular regeneration, and reduced vascular leak and edema.

### Example 3

### Efficacy of Connexin 43 Modulating Agent in Treating Wounds on mVLU Subjects

A 10-week randomized, parallel group, dose-ranging, controlled, multi-center study was conducted to assess the efficacy and safety of two dose concentrations of the Polynucleotide Formulation (1.0 mg/mL and 3.0 mg/mL) plus standard of care compression bandaging (SOC) vs. Polynucleotide Formulation Vehicle (poloxamer 407 gel) plus SOC (“Vehicle”) in subjects with a VLU. An additional SOC-alone arm was included in order to compare healing with Vehicle-treated subjects.

The primary objective of the study was to determine whether the Polynucleotide Formulation (1.0 or 3.0 mg/mL) improved healing of VLU. Percent surface area change of the reference VLU (RVLU) at 10 weeks was the primary endpoint of the study. Key secondary endpoints were incidence of complete RVLU closure and time to complete RVLU closure in the 10-week treatment period, both acceptable regulatory endpoints for registration studies.

A two-week Screening Period was designed to determine whether subjects were eligible to proceed to the treatment period of the study. The Investigator selected one RVLU at the first study visit (in patients with multiple VLU, this was the largest lesion that met the eligibility criteria for the study). Key eligibility criteria were patient age >18 years; confirmed venous insufficiency by venous duplex ultrasonography; non-infected, full thickness well-circumscribed VLU located above the malleolus; an ankle brachial index >0.80; and a VLU between 2 and 20 cm² at the end of the screening period. Centralized review of the RVLU photos was performed by the Medical Monitor during this period to supplement the Investigators' assessments of patient eligibility for randomization.

As SOC treatment, all subjects received multi-layer high compression bandaging (Coban™ 2; 3M) from the first screening visit until the end of the Treatment Period and for up to 2 weeks after the first incidence of RVLU closure was noted.

Eligible subjects proceeded to the Treatment Period and all (except those in the SOC-alone group) were assigned to double-blind treatment in one of three dose arms ((1) 3.0 mg/mL Polynucleotide Formulation comprising 3.0 mg/mL Polynucleotide (“3.0 mg/mL Polynucleotide Formulation” or “3.0 mg/mL”), (2) 1.0 mg/mL Polynucleotide Formulation, or (3) Vehicle). Visits were conducted once per week. Subjects progressed to the Post Treatment Period for up to 12 weeks of follow-up if the RVLU closed completely; otherwise subjects were discharged from the study after the Treatment Period, except SOC-alone subjects who could progress to up 10 weeks of open-label treatment with 3.0 mg/mL Polynucleotide Formulation.

313 subjects met the eligibility criteria and were randomized to the four treatment groups with 92, 97, 91 and 33 subjects assigned to the 3.0 mg/mL, 1.0 mg/mL, Vehicle and SOC-alone treatment groups, respectively. The average age of the 313 randomized subjects was 61.6 years (range 27.0 to 92). The mean BMI was 31.2 m/kg² (range 16.0-45.7).

All 313 randomized subjects were included in the Intention-To-Treat (ITT) population. The Safety Population (SP) population was identical to the ITT population. Thirty-one subjects were excluded from the ITr population by the Study Management Committee, resulting in a Per Protocol (PP) population of 282 subjects with 87 (94.6%), 83 (85.6%), 85 (93.4%) and 27 (81.8%) in the 3.0 mg/mL, 1.0 mg/mL, Vehicle and SOC-alone groups, respectively. All analyses presented below were performed on the entire IIT population.

The study included both sVLU and mVLU subjects, enrolled at random. In the more severe mVLU population (defined by subjects with more than one VLU), both raw and modelled data show a dose response for complete wound healing, and clinically significant deltas between the 1.0 mg/mL and 3.0 mg/mL dose concentrations of SEQ ID NO: 1 and Vehicle. As shown in Table 7 below, the raw values contrast between 3.0 mg/mL Polynucleotide Formulation and Vehicle is clinically significant at a 25% delta with mVLU subjects treated with 3.0 mg/mL showing a greater than 2.4-fold improvement in healing over vehicle (a 143% increase in wound healing), and is nearly significant at p=0.0658. Analysis with multiple-covariate logistic regression also shows a dose response and clinically significant differences between both active doses and Vehicle, and the difference between 3.0 mg/mL Polynucleotide Formulation and Vehicle was statistically significant at p=0.0127. Further analysis with a two-covariate logistic regression model was also statistically significant at p=0.042 (showing a 27.5% delta between 3.0 mg/mL and Vehicle, and a greater than 5.4-fold improvement in healing over vehicle (a 443% increase in wound healing)). This model contained ulcer duration, ratio of baseline wound circumference to area, wound surface area reduction during run-in, and baseline wound circumference (with only the latter two being statistically significant in the model).

TABLE 7
Multiple VLU Subject Data

| | | | | | 3.0 mg/mL vs. | |
|---|---|---|---|---|---|---|
| | | | | | Vehicle | |
| DATA | 3.0 | 1.0 | | | | P- |
| ANALYSIS | mg/mL | mg/mL | Vehicle | SOC | Delta | Value |
| RAW DATA | 42.3% | 38.7% | 17.4% | 25.0% | 24.9% | 0.0658 |
| LOGISTIC | 41.3% | 34.4% | 7.6% | 16.0% | 33.7% | 0.0127 |
| REGRESSION | | | | | | |

The logical regression in Table 7 was obtained by fitting logistic model with (a) treatment, (b) VLU status (multiple vs. single) and (c) all other retained covariates including interactions of treatment with BMI and age, to complete closure outcome using all data).

The raw value analysis shown above in Table 7 was not adjusted for covariates. The logistic regression data was obtained from a model that contains treatment, multiple VLU status, treatment by VLU interaction, and retained covariates, including age and BMI.

In sum, analysis of the results of the study using statistical models recommended by the FDA in its June 2006 Chronic Cutaneous Ulcer Guidance for Industry showed that treatment of VLU subjects using the Polynucleotide Formulation increased the incidence of complete healing of mVLU subjects and reduced the time to complete healing of mVLU when compared to both Vehicle and SOC. This demonstrates that the pharmaceutical formulations of this invention, which comprise an anti-connexin 43 modulator are suprisingly more effective at treating mVLU.

There were no safety issues identified in the safety population (i.e., all subjects who were randomized into the study whose RVLU was treated with at least one dose of investigational product or treatment, according to the randomization schedule; n=313), or in the mVLU population.

As shown in Table 8, the presence of multiple VLUs is associated with multiple covariates that are traditionally known as risk factors for poor healing. The data from this study show that in comparison with the single VLU wounds, multiple VLU lesions had significantly higher baseline area, circumference, necrotic tissue, HgA1c and wound duration; lower degrees of epithelialization and circumference/area ratio at randomization; and were found in patients with higher BMI. Other covariates were similar between single and multiple VLU subjects. It is noted, in this regard, that the overall unadjusted incidence of complete healing in the combined control groups in this study (i.e., the SOC-alone and Vehicle groups) was three times less for subjects with multiple ulcers (20.5%) than for subjects with only a single VLU (62.4%). This difference is statistically significant (p=0.001; chi-squared test), supporting the observation that an ulcer on a subject with multiple VLU is harder to heal than a solitary VLU.

TABLE 8
COMPARISON OF COVARIATE VALUES OF SINGLE VS.
MULTIPLE VLU

| | mVLU | | sVLU | | |
|---|---|---|---|---|---|
| Covariate | deviation | std | deviation | std | P-value |
| Baseline area | 6.13 | 4.19 | 5.04 | 3.77 | 0.024 |
| BMI | 32.7 | 6.87 | 30.7 | 6.80 | 0.015 |
| Circumference | 11.62 | 4.53 | 10.13 | 4.40 | 0.006 |
| Necrotic tissue | 2.03 | 6.83 | 0.71 | 2.81 | 0.071 |
| HbA1c | 6.13 | 0.99 | 5.97 | 0.80 | 0.185 |
| Epithelialization | 19.71 | 21.37 | 26.18 | 27.03 | 0.024 |
| Circumference/Area | 2.34 | 0.86 | 2.59 | 1.09 | 0.05 |
| Duration >1 yr | 35.42 | | 25.81 | | 0.10 |
| (Fisher Exact) | | | | | |

The following analyses were performed on the ITT population: (1) a logistic model to evaluate the incidence of complete wound closure; (2) a proportional hazards (Cox) model to evaluate time to complete healing; and, (3) a linear model to evaluate wound surface area reduction. Results for the healing endpoints, showing that the Polynucleotide Formulation improved both the incidence of complete wound closure and the time to complete healing, are summarized below and in Table 9.

The 3.0 mglmL Polynudeotide Formulation was 81.5% more effective than Vehicle in incidence of complete RVLU closure (46.1% vs. 25.4%; p=0.0533), with the odds of healing being 151.3% greater in the 3.0 mg/mL group than the Vehicle group (OR=2.5132).

The 3.0 mg/mL Polynucleotide Formulation was also 98.7% more effective to Vehicle in time to complete RVLUclosure, with the odds of healing first for unhealed patients throughout treatment being nearly twice as high in the 3.0 mg/mL group than the Vehicle group (HR=1.9875, p=0.0712).

TABLE 9
Key Endpoints for ITT Population in the Study

| | Incidence of | Time to |
|---|---|---|
| Dose | Complete RVLU | Complete RVLU |
| Concentration | Closure | Closure |
| (mg/mL) | (Probability) | (Hazard) |
| 3.0 | 46.1% | 9.06 |
| 1.0 | 29.2% | 4.67 |
| 0.0 (Vehicle) | 25.4% | 4.56 |
| SOC-alone | 32.3% | 2.23 |
| 3.0 vs. Vehicle | p = 0.053 | p = 0.071 |
| Vehicle vs. SOC | p = 0.592 | p = 0.348 |

### Multiple VLU Treatment—Polynucleotide Formulation Treatment and Other Indicators of Responder Subjects

Indicators important in the logistic regression model validated the expected effect of the indicator on overall healing, e.g., baseline wound circumference (p=0.0016), ulcer duration (p=0.0042), wound surface area change during run-in (p=0.0150), etc. An important discovery from the statistical analyses is that Polynucleotide Formulation treatment interacted with three prognostic indicators of healing: multiplicity of VLU (mVLU), patient age, and BMI. For BMI, the comparison between the 3.0 mg/mL dose concentration vs. Vehicle, demonstrated improved odds in favor of 3.0 mg/mL in subjects with BMI less than 42, including a BMI of less than 40. The interactions of multiplicity of VLU and age with treatment indicate that subjects with more severe venous disease and predisposition to poor healing are optimal for demonstrating the therapeutic effect of the Polynucleotide Formulation over Vehicle. Also of note is that the raw data analysis for multiple VLU subjects showed a 24.9% difference between the 3.0 mg/mL dose concentration and Vehicle for incidence of complete healing (42.3% vs. 17.4%, respectively; p=0.0658), which is a surprisingly large difference for wound healing treatments. The logistic model-adjusted incidence of complete healing data showed a larger, 33.7% difference between the 3.0 mg/mL dose concentration and Vehicle (41.3% vs. 7.6%, respectively; p=0.0127).

The study demonstrated that the Polynucleotide Formulation was safe and well tolerated, and showed clinically meaningful efficacy with the 3.0 mg/mL Polynucleotide Formulation. It was surprisingly found that treatment with the Polynucleotide Formulation was particularly efficacious in more severely diseased patients with multiple VLUs. In summary, the study demonstrated that the 3.0 mg/mL dose concentration was safe and resulted in marked clinical activity and clinically meaningful efficacy with Polynucleotide Formulation compared to Vehicle or SOC, for example using the 3.0 mg/mL in more severely diseased patients with multiple VLU.

### Example 4

### Treating Responder mVLU Subjects

Clinical trials are conducted to confirm and demonstrate the safety, tolerability and efficacy of a formulation comprising 3.0 mg/mL or 10.0 mg/mL Polynucleotide in the treatment of mVLU subjects susceptible to treating with an anti-connexin modulating agent.

The harder-to-heal multiple venous ulcer population will be the focus of this study. Human test subjects are treated with suitable doses of a suitable anti-connexin 43 polynucleotide formulation applied to all VLU sites under occlusive compression bandages.

In order to treat responder subjects likely to respond to treatment with the anti-connexin 43 modulating agent of this invention, subjects who have multiple (unilateral or bilateral) VLU are included in the study. These subjects were demonstrated to be the most difficult to heal. As discussed above, it was demonstrated by Applicants that the overall unadjusted incidence of complete healing in the combined control groups (i.e., the SOC-alone and Vehicle groups) was three times less for subjects with multiple ulcers (20.5%) than for subjects with only a single VLU (62.4%).

The formulation may be any formulation of this invention. In one aspect the formulation is the 3.0 mg/mL Polynucleotide Formulation of Example 4. Plasma will be obtained for PK measurements pre-dosing and 5, 15, 30, 60, 120 and 240 minutes post-dosing.

Additional inclusion and exclusion criteria refinements will be based on age over 50 and BMI of less than, for example, 42.

The primary objective of this study is to confirm that the 3.0 mg/mL Polynucleotide Formulation plus compression bandaging as SOC can improve the incidence of complete wound closure for mVLU subjects compared to Vehicle plus SOC. The secondary objectives are to determine whether the 3.0 mg/mL Polynucleotide Formulation is safe and tolerable and if the 3.0 mg/mL Polynucleotide Formulation improves time to complete wound closure

Standard-of-care in this study will comprise clinical wound evaluation by the Investigator, irrigation of the lesion with warm tap water or normal saline. Cytotoxic solutions such as Betadine are prohibited, but brief washing with a mild antiseptic agent that is subsequently washed completely off the wound surface is permitted. Mechanical wound surface cleaning or debridement will also be used in SOC treatment as indicated.

The Protocol will require a clean, healthy-appearing wound bed prior to each application of Polynucleotide Formulation. The surface of the lesion should be freed of slough, exudate and devitalized tissue; however the skin edges should not be excised and therefore, the wound should not be enlarged by the procedure. The SOC treatment will also include application of a primary dressing to the wound surface (e.g., Allevyn™ Non-Adhesive Dressing; Smith & Nephew) and wrapping of the mid-foot to the upper calf with a multi-layer compression secondary dressing (e.g., Coban™ 2; 3M). The peri-wound skin may be treated with moisturizing, anti-fungal or corticosteroid.

Consented subjects with multiple VLU will enter a two-week screening period where baseline assessments and eligibility assessments will be performed, including: venous duplex ultrasound to exclude subjects without underlying venous insufficiency, histopathology to exclude subjects with carcinoma in the VLU, and wound measurements to exclude subjects whose VLU is having large changes in size. Centralized review of the VLU photographs will be performed by the Medical Monitor to supplement the Investigator's judgment of eligibility for randomization, i.e., both the Medical Monitor and the Investigator must find that the subject is eligible in order for him/her to be randomized.

At the first visit, the Investigator will select a VLU that meets the eligibility criteria of the protocol to be the reference venous leg ulcer (RVLU). Each subject will have only one VLU selected as the RVLU. All other venous ulcers will be photographed and identified.

Subjects who continue to meet all of the inclusion criteria and have none of the exclusion criteria after completing the Screening Period will be randomized in a blinded fashion in a 1:1 ratio into either the Polynucleotide Formulation or Vehicle group. Either the Polynucleotide Formulation or Vehicle, will be applied weekly to the RVLU during the Treatment Period. Other VLU will not receive IP but will receive SOC treatment as prescribed by the protocol, including wrapping the entire lower study leg and proximal foot compression dressing provided for the study. Polynucleotide Formulation is applied topically around the inside edge of the ulcer to be treated and then applied to the remainder of the wound bed. This provides approximately 0.3 mg of product exposure per cm² of wound surface area. Due to the safety profile of the Polynucleotide Formulation, and the lack of any substantive safety issues as revealed by any nonclinical or clinical study, there are no special precautions or recommendations.

Treating only one reference ulcer with randomized investigational product accomplishes important objectives:

It enables use of well-defined endpoints comparable to those employed in other registration studies (incidence and time to complete wound closure of the reference ulcer).

Use of a single reference ulcer provides an objective endpoint that avoids the complication of grading multiple wounds. By contrast, including the criterion that all wounds heal may result in a patient being categorized as a failure even though, for an example, 3 of 4 wounds completely close at the end of the 12-week study period.

The presence of SOC-treated non-reference wounds could enable the in-patient comparison of vehicle vs. SOC treatment in patients who are randomized to receive vehicle. The power of this observation will be high since same-patient analysis removes any confounding effects of covariates such as, e.g., diabetes, age, concomitant medications and patient compliance.

For each subject, the Treatment Period will end:

At the first instance where 100% re-epithelialization of the RVLU is noted. In this instance the subject will immediately move to the Post-Treatment Period, or,

If the RVLU has not achieved 100% re-epithelialization after the completion of the T10 visit. These subjects will be contacted in 30 days to assess for any serious adverse events.

The Post-Treatment Period is designed to confirm RVLU complete closure, determine durability of closure and to continue to monitor for any serious adverse events. Complete wound closure is defined as 100% re-epithelialization without drainage confirmed at two visits, 14 (1) days apart. If the RVLU opens in the Post-Treatment Period the subject will exit the study.

Following completion of the study, the study is unblinded and the results analyzed. The results confirm that treatment of mVLU patients with 3.0 mg/mL Polynucleotide Formulation results in surprisingly high levels of complete closure of mVLU in this difficult to heal VLU population.

### Example 5

### Increased Expression of Connexin 26 and 30 in Chronic Wounds

The expression of connexins 26 and 30, in addition to connexin 43, was examined in patients with a variety of chronic wounds, including venous leg, diabetic foot or pressure ulcers. Wound edge punch biopsies were taken from a cohort of patients with venous leg, diabetic foot or pressure ulcers. Wound connexin expression in each patient was compared to that in a matched, non-wounded arm punch. Tissue was sectioned, stained and imaged by confocal microscopy using identical parameters per patient to permit quantification. Epidermal Cx43, 26 and 30 and dermal Cx43 were discovered to be strikingly up-regulated in every ulcer from all three wound types, indicating that connexin up-regulation is a common feature between different types of chronic wounds. This result supports the therapeutic targeting of Cx26 and Cx30, alone or in combination with Cx43, to promote cell migration and wound healing in chronic ulcers.

Connexins show dynamic changes in expression following acute wounding. In animal studies, Cx43 was shown to be naturally down-regulated in wound edge (WE) keratinocytes and fibroblasts as they become migratory, whilst Cx26 and Cx30 were up-regulated in the epidermal leading edge. (Goliger & Paul (1995), Wounding alters epidermal connexin expression and gap junction-mediated intercellular communication, Mol Biol Cell 6: 1491-501; Coutinho, et al (2003), Dynamic changes in connexin expression correlate with key events in the wound healing process, Cell Biol Int 27: 525-41; Mendoza-Naranjo et al. (2012a), Targeting Cx43 and N-cadherin, which are abnormally upregulated in venous leg ulcers, influences migration, adhesion and activation of Rho GTPases. PloS One 7: e37374; Mendoza-Naranjo, et al. (2012b), Overexpression of the gap junction protein Cx43 as found in diabetic foot ulcers can retard fibroblast migration, Cell Biol Int 36: 661-7. In biopsies from patients with mixed ulcers and DFUs, Cx43, 26 and 30 were detected at epidermal wound margins as well as in cells at some distance from the epidermal wound edge (WE) (Brandner, et al (2004), Connexins 26, 30, and 43: differences among spontaneous, chronic, and accelerated human wound healing, J Invest Dermatol 122: 1310-20), but the involvement of Cx regulation within the epidermis in chronic wound persistence has not been thoroughly investigated. The Cx status of the cells of the dermis may also be very important. Recently it has been reported that Cx43 expression in fibroblasts changes their cell-to-cell adhesion and cytoskeletal response during wound healing, with Cx43 up-regulation retarding their rate of migration (Mendoza-Naranjo et al., 2012b). Determining the levels of Cx expression in a variety of chronic wounds is an important step in our understanding of the link between Cx expression and impaired healing.

### Biopsy Acquisition, Preservation and Cryosectioning

Patients were eligible for study inclusion if they were over 18 yrs years and had an uninfected chronic wound present for at least 4 weeks, irrespective of current or previous treatments. Wound etiology was taken from the clinician's notes. Table 10 shows the clinical characteristics of the patients in this study.

TABLE 10
Clinical Characteristics

| | VLU (n = 19)¹ | DFU (n = 11) | PRU (n = 6)² |
|---|---|---|---|
| Age | 59 | 59 | 62 |
| | [31-79] | [48-82] | [34-88] |
| Male (%) | 63% | 64% | 83% |
| Wound Location | | | |
| Gaiter/lower leg (%) | 79% | NA | NA |
| Ankle (%) | 16% | NA | NA |
| Foot (%) | 5% | NA | NA |
| Dorsal foot (%) | NA | 46% | NA |
| Plantar foot (%) | NA | 36% | NA |
| Toe (%) | NA | 9% | NA |
| Ankle (%) | NA | 9% | NA |
| Sacral (%) | NA | NA | 67% |
| Malleolus (%) | NA | NA | 17% |
| Heel (%) | NA | NA | 17% |
| Median Wound Age | 6 (17)³ | 4 | 21 |
| (Months) | [1.5-108] | [1-26] | [4-48] |
| ≦3 months (%) | 35% | 46% | 0% |
| >3-≦6 months (%) | 24% | 27% | 17% |
| >6-≦12 months (%) | 12% | 18% | 17% |
| >12 months (%) | 29% | 9% | 66% |
| Median Wound Size | 9.9 | 6.6 | 7.0 |
| (cm²) | [2-113] | [0.56-22.2] | [3.4-40.5] |
| Diabetes Present (%) | 58% (17) | 100%⁴ | 60% (5) |
| IDD | 11% | 55% | 33% |
| HbA1c (%) | 10.4% (4) | 7.3% (5) | Unknown |
| | [6.5-13.5] | [6.4-9.9] | |
| BMI | 46 (12) | 34 | 27 |
| | [28-70] | [24-44] | [25-29] |

All values in Table 10: Median [range]; (n)=number of subjects with data available when lower than the full cohort. Abbreviations used in Table 10 include: VLU, venous leg ulcer; DFU, diabetic foot ulcer; PRU, pressure ulcer; IDD, insulin dependent diabetes; HbA1c, hemoglobin A1c; BMI, body mass index; NA, not applicable.

Wound edge biopsies of chronic wound tissue (VLU: n=19 patients; DFU: n=11; PRU: n=6) were obtained during the Outpatients Clinic visit by a single operator (TES) via a 4 mm full thickness punch biopsy taken from the visible WE. The biopsy side away from the open wound was marked with ink to keep the sample orientated throughout processing. Each patient also supplied a matched 4 mm punch biopsy of arm skin, providing unwounded baseline Cx expression levels.

All biopsies were immediately immersed in 4% paraformaldehyde for 24 hours and then transferred into 20% sucrose in phosphate buffered saline (PBS). Tissue blocks were embedded in optimal cutting temperature medium (OCT) (BDH—Poole, UK) and stored at −80° C. Frozen sections, 14 μm thick, were obtained using a Leica CM1900 UV cryostat and positioned on gelatine-coated slides.

### Immunohistochemistry

Frozen sections were defrosted and immersed in PBS to dissolve excess OCT. The tissue was permeabilized for 5 minutes in acetone and non-specific binding was blocked using PBS-lysine (0.1 M) over a 30 minute period. Primary antibodies were prepared in PBS-lysine (Cx43 1:4000 (Sigma—Poole, UK—C6219), Gap28H (Cx26) 1:200 (Diez et al. (1999), Assembly of heteromeric connexons in guinea-pig liver en route to the Golgi apparatus, plasma membrane and gap junctions, Eur J Biochem 262: 142-8), Cx30 1:200 (Invitrogen—Paisley, UK—71-2200), Smooth Muscle Actin 1:200 (Sigma—Poole, UK—A2547)). The tissue was incubated in a humid staining chamber with the primary antibody for 1 hour at room temperature. The tissue was washed with PBS-lysine for 3×5 minutes followed by application of the secondary antibody (Invitrogen—Paisley, UK—Alexa Fluor® 488 goat anti-rabbit 1:400 or Alexa Fluor® 568 goat anti-mouse 1:400) in conditions identical to those used with the primary antibody for 1 hour. Nuclei were stained using HOECHST (Sigma—Poole, UK—B-2883 and B-2261 1:50,000 in PBS) for a 5 minute period followed by 2×10 minute PBS washes. Coverslips were mounted using Citiflour® (Glycerol/PBS solution, Citiflour Ltd, London, UK).

### Confocal Microscopy

For the 4 mm biopsy samples an Olympus FV-1000 inverted confocal microscope was used to obtain 10× and 20× qualitative montage images of whole tissue sections and 40× quantitative images (epidermis and dermis) of the arm and wound. The 4 mm biopsies were examined (epidermis and dermis) across their diameter at three locations: at the WE, 1 mm from the WE, and at the far edge (FE). Hoescht was excited by a 405 nm, Alexa Fluor® 488 by a 488 nm and Alexa Fluor® 568 by a 565 nm wavelength laser.

### Image Quantification and Statistical Analysis

Cx quantification was carried out using ImageJ. Epidermal and dermal thresholds were kept constant between all images being set at 80 and 100-255 respectively with a recognized pixel threshold size of 2-infinity utilized for all images (Wang et al. (2007), Abnormal connexin expression underlies delayed wound healing in diabetic skin, Diabetes 56: 2809-17). In the epidermis Cx expression was related to the cell number as pixels/cell and in the dermis as pixels/μm².

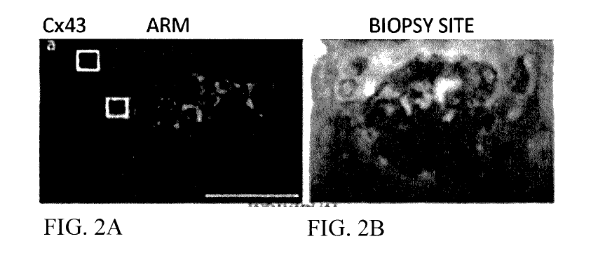

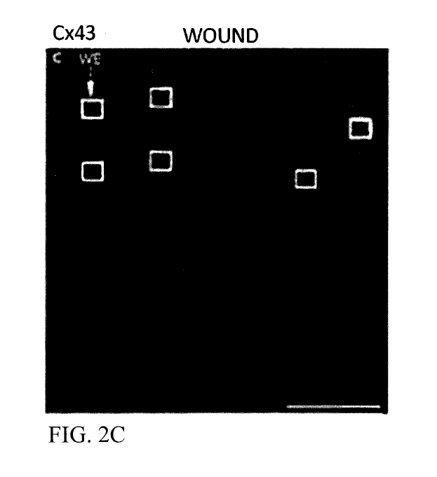

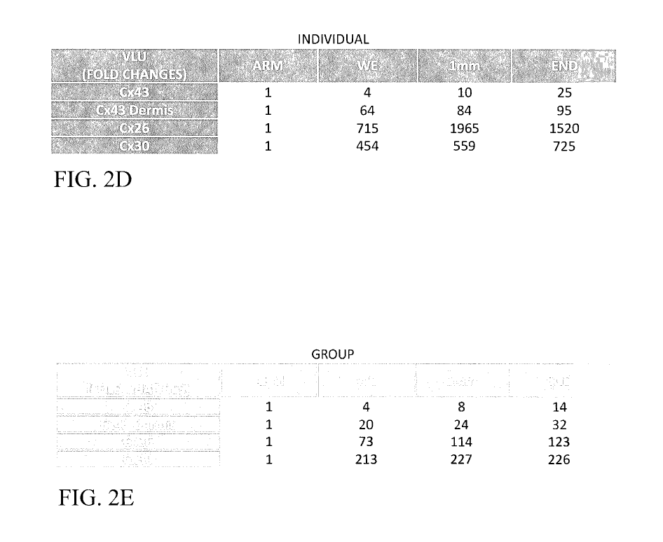

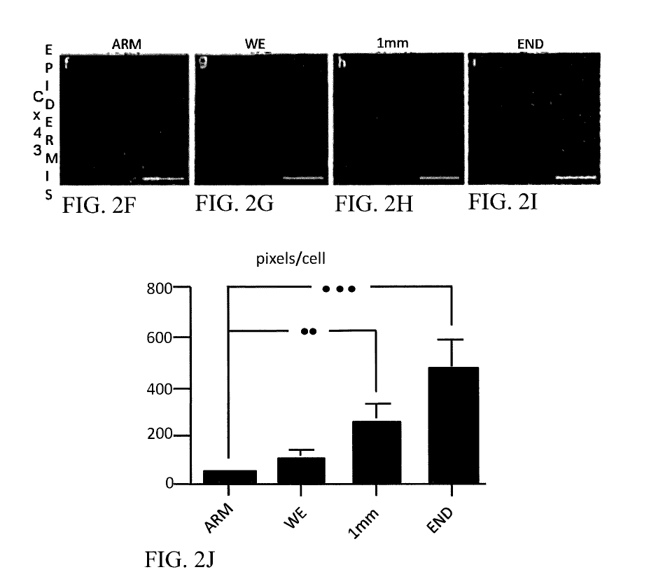

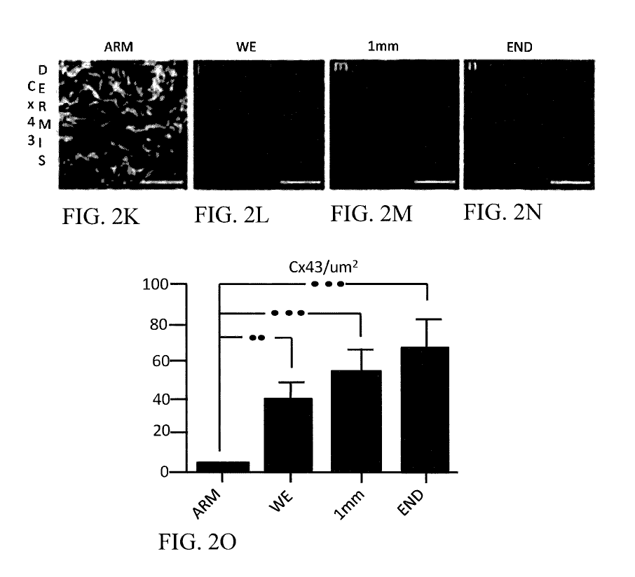

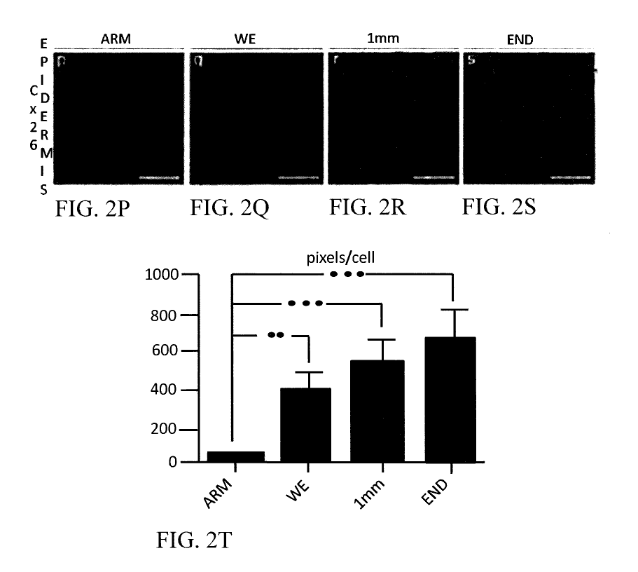

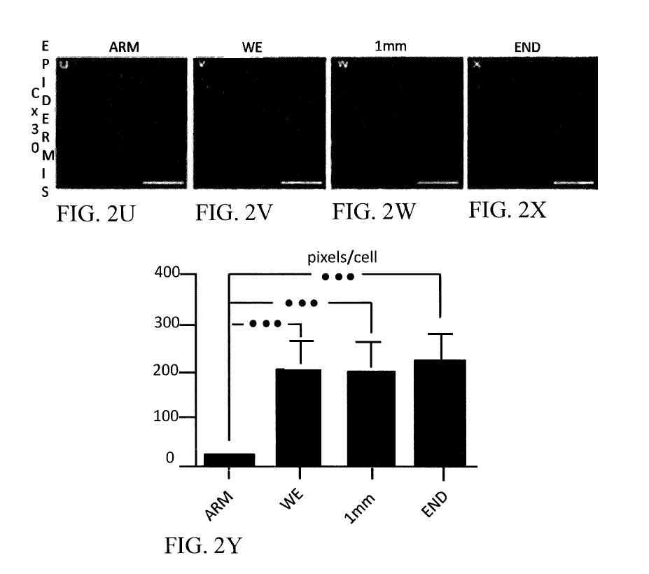

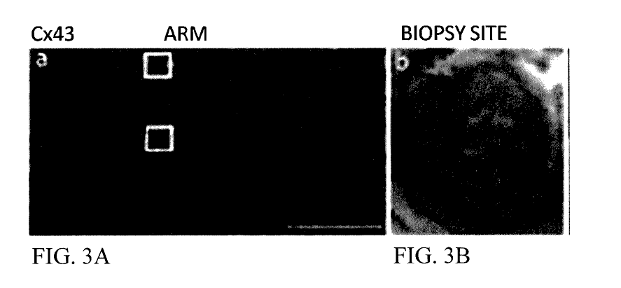

The data from the connexin measurements is presented in the results section as the “absolute Cx expression level” which was used for the statistical analysis (below) and is presented in the graphs (FIG. 2-4). The corresponding fold change data is in the tables as i) “fold difference of the group means”, this being the fold difference between the forearm biopsy group mean and the various wound location group means (WE, 1 mm, FE)); and ii) the “mean of the individual fold changes.” This was based on calculating each individual's unique fold difference by first normalizing their wound biopsy Cx expression to their matched forearm Cx level. Then a mean individual fold difference was calculated for each study group (i.e., this was the mean of the individually normalized Cx fold changes). This dataset gives an indication of how much the individual Cx fold differences could vary between patients.

### Statistical Analysis

The Cx expression data were analyzed using a two-way ANOVA, the two factors/variables being location (i.e., arm, WE, 1 mm from the WE, and FE) and patient.

The residuals were tested for normality using the Kolmogorov-Smimoff test; with a parametric distribution being assumed in all cases where the p-value=0.05. Normality was not reached in three groups: VLU Cx30, DFU Cx30 and DFU Cx43 epidermal values. These specific data sets were independently transformed using the natural log before analysis. A Dunnett's post-hoc test compared all three wound measurements back to the reference group, i.e., arm values. Significance was taken at values p=0.05.

### Features of Chronic Wound Biopsies

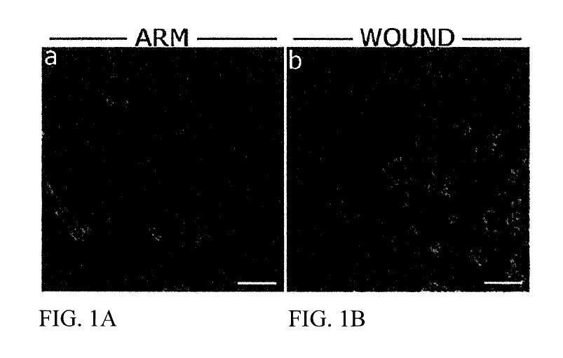

The histology of chronic wound biopsies varied but consistent features were identified that distinguished them from healthy tissue as seen in FIG. 1, a representative VLU. These include increased depth to the epidermal rete pegs, a greater number of blood vessels, and a large abundance of neutrophils both within dead tissue at the WE and throughout the dermis.

In acute wounds the early hallmark of active healing is the formation of a thin keratinocyte tongue at the WE, indicating the start of re-epithelialization. These cells have a migratory phenotype and crawl forward across the wound bed. None of the DFU biopsies presented with a thinning of the epidermal WE. However, a thinning tongue of WE keratinocytes was identified in some VLUs ( 6/19 biopsies), which may represent the beginning of healing or attempts to heal in some wounds. In the pressure ulcer (PRU) cohort, two out of the six wounds examined had this feature.

Biopsies from Venous Leg Ulcers

Biopsies from VLUs revealed several consistent features (FIG. 2). The epidermis of the 4 mm biopsies were typically hyper-thickened, increasing in depth with distance from the WE. However, in some samples, the epidermis consistently thinned towards the WE and had the appearance consistent with a migratory phenotype, as noted above. The epidermal expression of Cx43 and Cx30 were increasingly elevated along the length of the biopsy as the epidermis became increasingly thickened upon moving away from the WE, whereas Cx26 was uniformly elevated in the epidermis along the biopsy. The levels of Cx43 at the epidermal WE of these biopsies, whilst having a 4-fold higher absolute group mean than that seen in the normal unwounded arm tissue were not significantly different. However, 1 mm from the WE the absolute group mean elevation in Cx43 was 8-fold higher than the reference arm tissue and highly statistically significant (p<0.01), whilst on the far edge (FE) of the biopsy the increase was on average 14-fold and very highly statistically significant (p<0.001). Cx26 and Cx30 are normally expressed at relatively low levels in the intact skin in comparison to Cx43 but were reported to be increased in hyper-proliferative human keratinocytes (Rivas et al. (1997), Identification of aberrantly regulated genes in diseased skin using the cDNA differential display technique, J Invest Dermatol 108: 188-94; Labarthe et al. (1998), Upregulation of connexin 26 between keratinocytes of psoriatic lesions, J Invest Dermatol 111: 72-6). These two proteins had a many-fold greater elevation than that observed for Cx43 in the chronic wound tissues examined. For example, epidermal WE Cx30 was significantly elevated by an average of 213-fold rising to 226-fold at the thicker, FE location (p<0.01 and p<0.001). Cx26 was also significantly elevated, 73-fold at the WE epidermis, rising to 123-fold at the FE of the biopsy when compared to the matched, intact reference tissue (p<0.001).

A common feature within the dermis of VLUs was an increased number of blood vessels (FIG. 1) along with a loss of the auto-fluorescent extracellular matrix in the upper third of the dermis (FIG. 2c). Dermal fibroblasts do not express Cx26 or Cx30 but do express Cx43 and this was significantly elevated across the dermis, increasing by 20-fold at the WE and 32-fold at the FE when compared to matched, unwounded tissue (p<0.01 and p<0.001). The corresponding mean of the individual normalized fold changes for each Cx are found in FIG. 2d.

Biopsies from Diabetic Foot Ulcers

Biopsies from DFUs also had common features (FIG. 3). The epidermis was hyper-thickened but, unlike VLUs, this was more uniform in DFU samples. None of the biopsies showed any signs of thinning towards the WE and had no appearance of healing (FIG. 3c). Like the VLU, the DFU also had elevated levels of Cx expression but this was fairly consistent across the length of the biopsy. The Cx43 absolute group mean was elevated by 9-fold at the WE and 7-fold at the FE when compared to the unwounded forearm (both p<0.001). Cx26 and Cx30 were also significantly increased, by 62-fold (p<0.05) and 201-fold (p<0.001) respectively, at the WE and 64-fold (p<0.05) and 115-fold (p<0.001) at the FE side of the biopsy.

The dermis of the DFUs was distinctly different from that of the VLUs, as in many cases it lacked any signs of auto-fluorescent signal from the fibers of the dermal extracellular matrix or, if auto-fluorescence remained, the organizational pattern was absent. This suggested that a large proportion of the native collagen and elastin had either been degraded or was no longer being arranged into mature fibrils. The DFU dermis featured significantly increased levels of Cx43, by an average of 20-fold at the WE and 18-fold on the FE of the biopsy (p<0.05 and p<0.01). These data and the means of the individual normalized fold changes for each Cx are found in FIG. 3d.

Biopsies from Pressure Ulcers (PRUs)

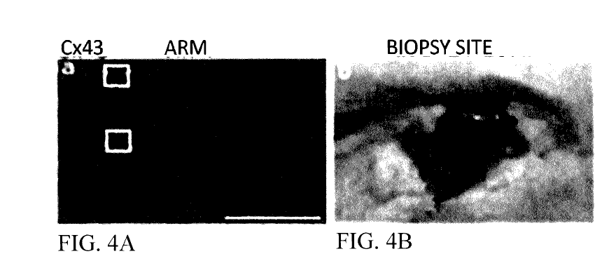

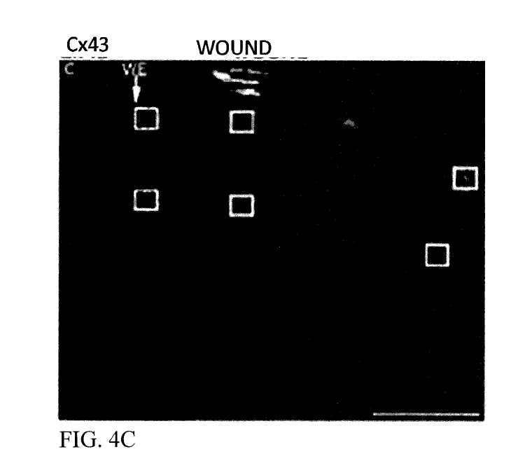

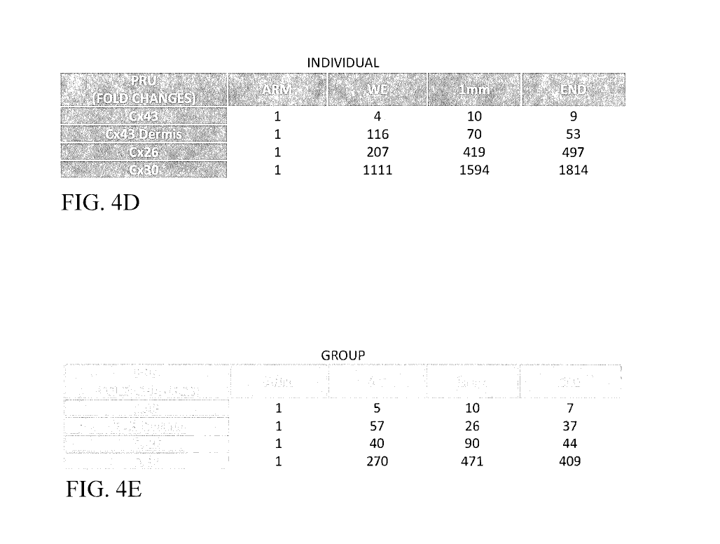

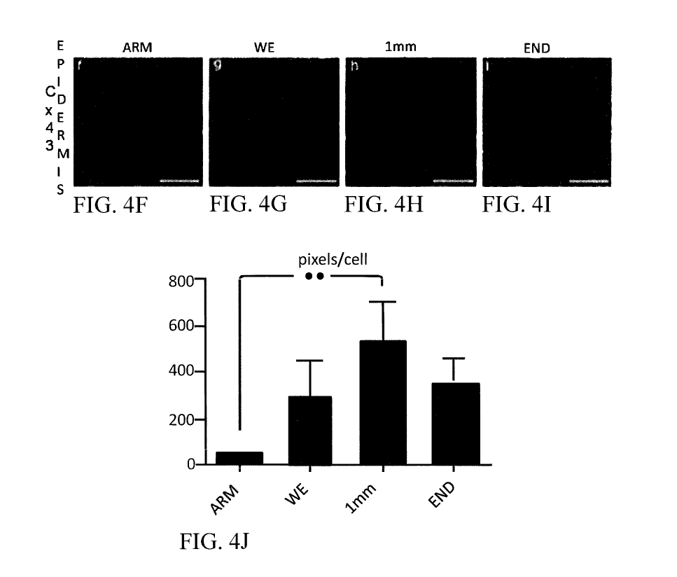

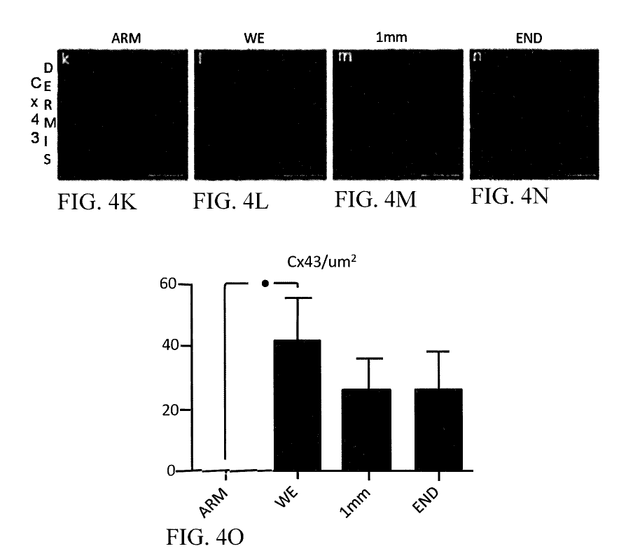

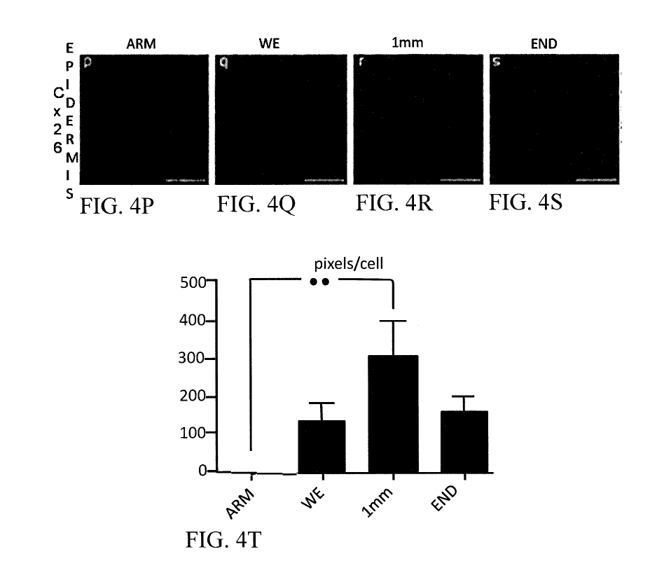

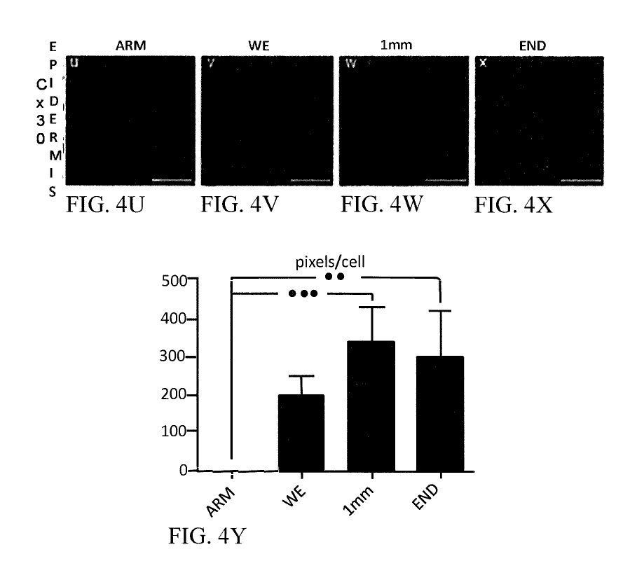

Biopsies from PRUs were variable in their appearance (FIG. 4). The epidermis was typically thickened along the length of the biopsy with the formation of deep rete pegs. The degree of healing was variable, manifesting in some instances as a thinning tongue of WE epidermis and reminiscent of the VLUs. Again, Cx expression was elevated in the epidermis but unevenly so at the WE. The Cx43 absolute group mean was significantly increased at 1 mm by 10-fold (p<0.01), whilst Cx26 was elevated 90-fold and Cx30 by 471-fold when compared to the low baseline levels found in intact arm skin (p<0.01 and p<0.001).

The dermis of the PRUs could be distinguished from the VLU and DFU by the consistent presence of an auto-fluorescent signal from the extracellular matrix. Cx43 expression was significantly increased on average 58-fold at the WE and 37-fold on the FE side of the wound (p<0.05). These data and the means of the individual normalized fold changes for each Cx are found in FIG. 4d.

### Distribution of Epidermal Connexin (Cx) Overexpression

The distribution of connexins within the epidermis varied along the length of the biopsies and with depth corresponding to the varying layers of the epidermis. The deep rete pegs were characterized by a dominance of Cx26 and 30. In some regions, a large proportion of the cell membrane appeared to be taken up by connexins, giving the staining a “fish scale” appearance.

As discussed above, it has therefore been demonstrated that a statistically significant, substantial up-regulation of three connexin gap junction proteins in VLUs, DFUs and PRUs, i.e., epidermal connexin 26, connexin 30 and connexin 43 and dermal connexin 43. Precise spatial and temporal control of connexin proteins has been shown to be integral to the regular wound reparatory process, where down-regulation of the Cx43 at the wound edge is correlated to keratinocyte and fibroblast migration. The Cx misregulation we have identified here may serve to slow healing and/or prolong ulceration (Wang et al. 2007).

### Epidermal Over-Expression of Connexin26 (Cx26) and Connexin30 (Cx30)

To date most research on Cx dynamics throughout wound repair has focused on elucidating the role of Cx43. Cx26 and Cx30 are usually only detected at very low levels within the intact interfollicular epidermis but are significantly up-regulated post-wounding within the migratory epidermal leading edge (Coutinho et al., 2003). Examination of these proteins within chronic wound tissue found them both to be significantly over-expressed across the entirety of the epidermis, which correlates with a variety of skin proliferative conditions. For example, up-regulation of Cx26 and/or Cx30 has previously been reported in psoriasis (Lemaitre, et al. (2006), Connexin 30, a new marker of hyperproliferative epidermis, Br J Dermatol 155: 844-6; Lucke et al. (1999), Upregulation of connexin 26 is a feature of keratinocyte differentiation in hyperproliferative epidermis, vaginal epithelium, and buccal epithelium, J Invest Dermatol 112: 354-61), warts (Lucke et al., 1999) and a variety of genetically inherited conditions that lead to skin abnormalities, such as Porokeratosis of Mibelli (Hivnor et al. (2004), Gene expression profiling of porokeratosis demonstrates similarities with psoriasis, J Cutan Pathol 31: 657-64), and Clouston syndrome (Lemaitre et al, 2006). Based on the data from the experiments as descried herein, it has been recognized that the common phenotypic factor between these syndromes and chronic wounds is keratinocyte hyper-proliferation.

Keratinocyte proliferation and differentiation is misregulated in DFUs and VLUs (Stojadinovic et al, 2008; Usui et al, 2008). In VLUs there is a loss of cell cycle control, along with the mis-expression of activation and differentiation pathways (Stojadinovic et al. (2008), Deregulation of keratinocyte differentiation and activation: a hallmark of venous ulcers, J Cell Mol Med. 12: 2675-90). In DFUs, keratinocytes at the WE are hyper-proliferative, independent of ulcer edge thickness. Interestingly this extends into the non-ulcerated region, with tissue of a histologically “normal” phenotype staining strongly for the cell proliferation marker, Ki67 (Usui et al. (2008), Keratinocyte migration, proliferation, and differentiation in chronic ulcers from patients with diabetes and normal wounds, J Histochem Cytochem 56: 687-96). The over-expression of Cx26 and Cx30, as detected along the entire length of the 4 mm punch biopsy independent of ulcer type, could reflect their direct involvement in the epidermal thickening.

Studies of acute incisional wound healing in transgenic mice, where Cx26 is ectopically expressed within keratinocytes, showed delayed wound healing and a hyper-proliferative epidermal state (Djalilian et al. (2006, Connexin 26 regulates epidermal barrier and wound remodeling and promotes psoriasiform response, J Clin Invest 116: 1243-53. After 21 days post-wounding only 42% of the heterozygous mice had healed whilst full epidermal barrier restoration was seen in all wild-type animals by day 14 (Djalilian et al, 2006). Alternatively, Cx26 and 30 may be markers of hyper-proliferation with their expression predominantly influencing cellular differentiation.

### Epidermal and Dermal Connexin43 (Cx43) Over-Expression

In this Example, human chronic wounds of the three major etiologies were found to have abnormally high connexin expression at the WE. By way of example, Cx43 WE expression was 9 times greater in the DFU cohort than basal, unwounded skin levels. When this is considered in the context of the substantial preclinical data that links delayed healing with elevated Cx43 expression, it strongly indicates that the up-regulation of this protein is likely a common feature of chronic wound pathology.

In summary, it has been demonstrated that the over-expression of Cx26, Cx30, and Cx43 in the epidermis and that of Cx43 in the dermis of ulcer biopsies is a signature feature of chronic wounds, identified in all patients irrespective of ulcer type, i.e., VLU, DFU or PRU.

### FIGURE LEGENDS

FIG. 1. Blood vessels in VLUs. Blood vessel staining (green) in a representative (a) intact arm skin biopsy and (b) VLU. Chronic wound tissue is characterized by an enhanced number of dermal blood vessels. Scale bars—100 and 500 μm respectively. Cx43=Red; Blood vessels/alpha smooth muscle actin=Green; Nuclei and autofluorescent extracellular matrix=Blue. VLU, venous leg ulcer; Cx, connexin.

FIG. 2. Cx43, Cx26 and Cx30 expression in VLUs. (a&c) Location of quantification sites. (b) Representative VLU. (d-e) Mean of the individual and group Cx fold changes compared directly to the reference arm values. (f-y) Cx43, 26 and 30 expression with associated summary graphs for the WE, 1 mm from the WE and at the FE. Scale bar—10× Montages 1000 pun; 40× Images 100 mun. Cx43, 26 and 30=Green; Nuclei=Blue. ** p<0.01; *** p<0.001. Error bars—Mean+/−SEM (Epidermis—n=19 except FE=14; Dermis—n=17 except WE=15; FE=13) VLU, venous leg ulcer; Cx, connexin; WE, wound edge; FE, far edge.

FIG. 3. Cx43, Cx26 and Cx30 expression in DFUs. (a&c) Location of Cx43 quantification sites. (b) Representative DFU. (d-e) Mean of the individual and group Cx fold changes compared directly to the reference arm values. (f-y) Cx43, 26 and 30 expression and associated summary graphs for the WE, 1 mm from the WE and at the FE. Scale bar—10× Montages 1000 μm; 40× Images 100 μm. Cx43, 26 and 30=Green; Nuclei=Blue. * p<0.05; ** p<0.01; *** p<0.001. Error bars—Mean+/−SEM (Epidermis—n=11 except FE=8; Dermis—n=6). DFU, diabetic foot ulcer; Cx, connexin; WE, wound edge; FE, far edge.

FIG. 4. Cx43, Cx26 and Cx30 expression in PRUs. (a&c) Location of Cx43 quantification sites. (b) Representative PRU. (d-e) Mean of the individual and group Cx fold changes compared to directly to the reference arm values. (f-y) Cx43, 26 and 30 expression and associated summary graphs for the WE, 1 mm from the WE and at the FE. Scale bar—10× Montages 1000 μm; 40× Images 100 μm. Cx43, 26 and 30=Green; Nuclei=Blue. * p<0.05; ** p<0.01; *** p<0.001. Error bars—Mean+/−SEM (Epidermis and dermis—n=6 except FE=5). PRU, pressure ulcer; Cx, connexin; WE, wound edge; FE, far edge.

### Example 6

### Efficacy of Connexin 43 Modulating Agent in Treating Wounds on mDFU Subjects

A Phase 2, 12-week, randomized, four-arm, double-blind, vehicle-controlled, dose-ranging, multi-center study was conducted to assess the efficacy and safety of three dose concentrations of the Polynucleotide Formulation (3.0 mg/mL, 10.0 mg/mL, and 30 mg/mL) compared to Vehicle when applied to diabetic foot ulcers. Throughout the entire duration of the study, from the start of Screening until the end of each subject's participation, each subject received best practice standard of care (SOC) for diabetic foot ulcers. The SOC consisted of debridement/cleaning and dressing the wound, then off-loading with a removable cast walker (RCW). During the Treatment Period of the study, the assigned treatment was applied to the reference diabetic foot ulcer (RDFU) twice weekly in addition to the SOC.

The primary objective of the study was to evaluate use of the Polynucleotide Formulation (3.0 mg/mL, 10.0 mg/mL, and 30 mg/mL) to improve healing of DFU. Endpoints included the Incidence of Complete RDFU Closure (Primary Endpoint) and Time to Complete RDFU Closure within the 12-week treatment period, both acceptable regulatory endpoints for registration studies. A key secondary endpoint was Percent Surface Area Change of the RDFU within the 12 weeks.

A two-week Screening Period was designed to determine whether subjects were eligible to proceed to the treatment period of the study. For eligible patients, the Investigator selected one RDFU at the start of the Screening Period (in patients with multiple DFU, this was the largest DFU that met the eligibility criteria for the study). The Screening Period was designed to exclude diabetic foot ulcers that had large changes in size and to exclude subjects who were non-compliant with the standard-of-care regime. Centralized review of RDFU photographs was performed during this period to assess the wound appearance and confirm eligibility.

In the parallel group phase of the trial, eligible subjects proceeded to the Treatment Period and all were assigned to double-blind treatment in one of four treatment arms [(1) 3.0 mg/mL Polynucleotide Formulation comprising 3.0 mg/mL Polynucleotide (“3.0 mg/mL Polynucleotide Formulation” or “3.0 mg/mL”), (2) 10.0 mg/mL Polynucleotide Formulation, (3) 30.0 mg/mL Polynucleotide Formulation, or (4) Vehicle]. In an earlier dose escalation phase eligible subjects (n=43) were assigned to one of two treatment arms until the study opened up to the Parallel Group Phase. The dose-rising safety assessment phase began with the 3.0 mg/mL Polynucleotide Formulation, proceeded to the 10.0 mg/mL Polynucleotide Formulation, and concluded with the 30.0 mg/mL Polynucleotide Formulation, during which no safety issues or concerns were observed.

A total of 168 subjects met the eligibility criteria and were randomized to the four treatment groups with 42, 43, 41 and 42 subjects assigned to the 3.0 mg/mL, 10.0 mg/mL, 30 mg/mL, and Vehicle, respectively. The assigned treatment or Vehicle was applied twice a week by clinical staff up to and including Day 84 of the 12-week Treatment Period. If an initial assessment of 100% re-epithelialization was made at a study visit prior to Day 84, the subject entered the Post-Treatment Period and was followed up for a further 14 days to confirm complete closure of the RDFU.

Only subjects whose RDFU was 100% re-epithelialized during the Treatment Period progressed to the Post-treatment Period. The duration of this period for each subject depended on the status of the RDFU at each assessment visit. For those subjects whose RDFU remain healed beyond the first 14 days of the Post-treatment Period, this follow-up extended up to a maximum of 12 weeks, to determine durability of RDFU complete closure and continued assessment of safety.

The study included both single DFU (sDFU) and multiple DFU (mDFU) subjects, enrolled at random. In the more severe mDFU population (defined by subjects with more than one DFU), both raw results and data from a simple statistical model comparing results for the 30 mg/mL dose compared to other groups combined (Vehicle with the lower 3.0 and 10 mg/mL doses) showed a positive treatment response for complete wound healing, and clinically significant increases in healing using the 30.0 mg/mL dose concentration of the Polynucleotide Formulation compared with other groups.

Multiple DFU subjects treated with the 30.0 mg/mL Polynucleotide Formulation showed a complete closure rate of 58.3%, while mDFU subjects treated with Vehicle had a 29% healing rate, i.e., a 100% improvement in healing was observed in the 30 mg/mL group. Expected 12-week DFU complete closure rates with standard-of-care treatment in clinical practice are understood to average about 30-35%. The raw values contrast between mDFU subjects treated with 30.0 mg/mL Polynucleotide Formulation and mDFU subjects in the other groups combined showed a 145% improvement in healing over combined (Vehicle+lower doses); with 30.0 mg/mL (showing 58% RDFU complete closure compared to 23.8% RDFU complete closure, p=0.0473).

In addition, further analysis using three simple statistical models taking into account only mDFU status, treatment, and mDFU status×treatment interaction showed that subjects treated with the 30.0 mg/mL Polynucleotide Formulation in comparison to mDFU subjects in the other groups combined had a clinically meaningful increase in complete wound closure (linear regression model; p=0.0554), a clinically meaningful faster time to complete wound healing (proportional hazards model; p=0.0459), and a clinically meaningful percent reduction in wound surface area (generalized linear model; p=0.036).

Additionally, the trial data show that wounds on mDFU patients are harder to heal than sDFU wounds (Odds Ratio=2.0638 for the complete healing in favor of sDFU; p=0.0133 for the differences in surface area reduction, also in favor of sDFU wounds). The study demonstrated that the Polynucleotide Formulation was safe and well tolerated, and showed clinically meaningful efficacy with the 30.0 mg/mL Polynucleotide Formulation and that treatment with the Polynucleotide Formulation was particularly efficacious in resistant wounds on patients with multiple DFUs.

The present invention is not limited by the aforementioned embodiments. It will occur to those ordinarily skilled in the art that various modifications may be made to the disclosed embodiments with-out diverting from the concept of the invention. All such modifications arc intended to be within the scope of the present invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of a Patent Office unless such statement is specifically and without qualification or reservation expressly adopted in a responsive writing by Applicants.

1. A method of treating a resistant lesion on a subject, the method comprising administering to the subject a composition comprising a therapeutically effective amount of a connexin protein modulating agent.

2. A method according to claim 1, wherein said connexin protein modulating agent is selected from a connexin 43 polynucleotide, a connexin 30 polynucleotide or a connexin 26 polynucleotide.

3. A method according to claim 2, wherein said polynucleotide is an antisense polynucleotide.

4. A method according to claim 3, wherein said antisense polynucleotide comprises a sequence selected from SEQ. ID. NOS: 1-3, SEQ. ID. NO.21 or SEQ. ID. NO.22-23 or from about 12 to 40 nucleotides complementary to connexin 43 mRNA, connexin 26 mRNA or connexin 30 mRNA.

5. A method according to claim 3, wherein said antisense polynucleotide is selected from:
(SEQ ID NO: 1)
GTA ATT GCG GCA AGA AGA ATT GTT TCT GTC;
(SEQ ID NO: 2)
GTA ATT GCG GCA GGA GGA ATT GTT TCT GTC;
and,
(SEQ ID NO: 3)
GGC AAG AGA CAC CAA AGA CAC TAC CAG CAT.

6. A method according to claim 3, wherein the antisense polynucleotide has from about 12 to about 35 nucleotides and has at least about 90 percent homology to a connexin 43 mRNA.

7. A method according to claim 1, wherein the composition comprises at least about 1.0 mg/mL of said anti-connexin agent and the anti-connexin 43 agent is an antisense polynucleotide.

8. The method of claim 1 wherein the connexin 43 modulating agent is a connexin mimetic peptide comprising a portion of an extracellular loop of connexin 43.

9. A method of claim 1, wherein the connexin 43 modulating agent comprises a connexin 43 peptide comprising SEQ. ID. NO: 10.

10. A method according to claim 1, wherein the composition comprises about 0.01 to about 100 mg/ml of an anti-connexin 43 peptide or anti-connexin 43 peptidomimetic.

11. The method of claim 9 wherein the connexin 43 modulating agent is a connexin mimetic peptide comprising any one of SEQ ID NO:8 or SEQ ID NO:9.

12. A method according to claim 1, wherein the subject is a mammal.

13. A method according to claim 10, wherein the mammal is a human.

14. A use according to claim 3, wherein said composition is formulated to provide sustained release of the antisense polynucleotide.

15. A method according to claim 1, wherein the composition further comprises a pharmaceutically acceptable vehicle comprising a gel.

16. A use according to claim 13 in which the gel is a nonionic polyoxyethylene-polyoxypropylene copolymer gel.

17. A use according to claim 14, wherein the gel is a pluronic gel.

18. A use according to claim 15, wherein the pluronic gel is poloxamer 407.

19. The method according to claim 13 wherein the gel is a thermoreversible gel.

20. The method of claim 1 wherein the connexin 43 modulating agent is administered more than once.

21. The method of claim 1, wherein the connexin 43 modulating agent is administered every 12 hours, from once every 1 to 2 days to once every 7 days, once biweekly, and once per month.

22. The method according to claim 1, wherein the connexin 43 modulating agent is administered about once per week.

23. The method according to claim 1, wherein the connexin 43 modulating agent is administered more than once a week.

24. The method of claim 1, wherein the connexin 43 modulating agent is administered bi-weekly.

25. The method according to claim 20, wherein the connexin 43 modulating agent is administered for up to four, six, eight, ten, twelve, fourteen, sixteen, eighteen, twenty, twenty-two, twenty-four or twenty-six weeks.

26. The method of claim 1 wherein a repeat application of the connexin 43 modulating agent is administered in the event that healing of the lesion slows or is stalled.

27. The method of claim 1 wherein the resistant lesion is a venous leg ulcer.

28. The method of claim 1 wherein the resistant lesion is a diabetic foot ulcer or a pressure ulcer.

29. The method of claim 1 wherein the resistant lesion is an mVLU or mDFU characterized by surface area reduction of less than about 25-30% in a two week pretreament period with a standard of care treatment.

30. The method of claim 29, wherein the lesion heals by less than about 25% as measured by surface area reduction.

31. The method of claim 30, wherein the lesion heals by less than about 20% as measured by surface area reduction.

32. The method according to claim 1, wherein the resistant skin lesion is an mVLU or mDFU characterized at least in part by healing not more than about 10-20% during a pretreatment or run-in period with a standard-of-care treatment.

33. The method according to claim 1, wherein the resistant skin lesion is an mVLU or mDFU characterized at least in part by healing not more than about 10-15% during a pretreatment or run-in period with a standard-of-care treatment.

34. The method according to claim 1, wherein the resistant skin lesion is an mVLU or mDFU characterized at least in part by healing not more than about 17.5% during a pretreatment or run-in period with a standard-of-care treatment.

35. The method according to claim 1, wherein the resistant skin lesion is an mVLU or mDFU characterized at least in part by healing not more than about 20-30% during a pretreatment or run-in period with a standard-of-care treatment.

36. The method according to claim 1, wherein the resistant skin lesion is an mVLU or mDFU characterized at least in part by healing not more than about 20-25% during a pretreatment or run-in period with a standard-of-care treatment.

37. The method of claim 1 wherein the resistant lesion is an mVLU or mDFU characterized by a reduction in surface are of not more than about 30-50% in a four-week period of treatment with a standard of care.

38. The method of claim 37, wherein the reduction in surface are is not more than about 30%.

39. The method of claim 38, wherein the reduction in surface are is not more than about 25%.

40. The method of claim 1 wherein the resistant lesion is characterized by having less than about 10% epithelializing tissue or by being at least 8 cm² in size with a minimal degree of epitheliazation.

41. The method of claim 1 wherein the subject has more than one lesion on either or both legs and/or feet.

42. A method of treating a venous leg ulcer on a subject having multiple venous leg ulcers, the method comprising administering to the ulcer on the subject a composition comprising a connexin 43 modulating agent in amounts effective to promote healing of the ulcer.

43. A method of treating a venous leg ulcer on a subject having more than one venous leg ulcer, the method comprising administering to the subject a connexin 43 oligodeoxynucleotide present at a concentration of at least about 1 mg/mL.

44. A method of treating a diabetic foot ulcer on a subject having multiple diabetic foot ulcers, the method comprising administering to the ulcer on the subject a composition comprising a connexin 43 modulating agent in amounts effective to promote healing of the ulcer.

45. A method of treating a diabetic foot ulcer or a pressure ulcer on a subject having more than one diabetic foot ulcer or pressure ulcer, the method comprising administering to the subject a connexin 43 oligodeoxynucleotide present at a concentration of at least about 1 mg/mL.

46. The method of any one of claim 39 or 41, wherein the connexin 43 oligodeoxynucleotide is present at a concentration of at least about 1-3 mg/mL.

47. A method of determining whether to treat a subject having at least one venous leg ulcer with an connexin 43 modulating agent, the method comprising the steps of (a) determining one or more indicators selected from the group consisting of the subject's VLU status, age, and BMI measurement, and (b) treating the subject with a connexin 43 modulating agent based on the presence of one or more indicators selected from multiple VLUs, age over 50 or BMI measurement of less than 40.

48. A kit or an article of manufacture comprising package material containing a composition comprising a connexin 43 modulating agent in amounts effective for use in the method of claim 1 together with instructions for use in (a) treating a subject (a) having a resistant lesion; and/or (b) treating a subject having multiple venous leg ulcers; and/or (c) treating a subject having multiple diabetic foot ulcers; and/or (c) treating a subject having multiple pressure ulcers.

49. A method of promoting the surface area reduction of a resistant skin lesion, the method comprising administering the ulcer on the subject a composition comprising a connexin 43 modulating agent in amounts effective to promote epithelialization.

50. The method according to claim 1, wherein the subject is characterized at least in part by the presence of mVLU.

51. The method according to claim 1, wherein the subject is characterized at least in part by the presence of mDFU or multiple pressure ulcers (mPU).

52. The method according to claim 1, wherein the resistant skin lesion is characterized at least in part by showing a linear lesion advance of less than about 0.007 cm/day during a pretreatment or run-in period with standard-of-care treatment.

53. The method according to claim 1, wherein the resistant skin lesion is characterized at least in part by showing a linear lesion advance of less than about 0.05 cm/week during a pretreatment or run-in period with standard-of-care treatment.

54. The method according to claim 55, wherein the resistant skin lesion is characterized at least in part by showing a linear lesion advance of about 0.025 to 0.03 cm/week during a pretreatment or run-in period with standard-of-care treatment.

55. The method according to claim 1, wherein the resistant skin lesion is >5 cm² (size) and/or has persisted for >6 months (duration).

56. The method according to claim 1, wherein the resistant skin lesion exhibits less than 10% epithelization.

57. The method according to claim 1, wherein the resistant skin lesion is an mVLU or mDFU characterized by not showing a surface area reduction of at least about 30% over a 2- to 4-week pretreatment or run-in period during with the subject is treated with a hydrogel (for example).

58. The method according to claim 58, wherein the hydrogel is selected from a Curasol hydrogel, Gentell hydrogel, or a poloxamer gel plus standard-of-care treatment.

59. The method according to claim 1, wherein the resistant skin lesion is characterized by low levels of mitotic activity, high levels of inflammatory cytokines and/or proteases, low levels of growth factors, and/or nearly senescent fibroblasts, in comparison to healing or acute wounds.


SEQUENCE LISTING (31 sequences)

SEQ ID NO: 1 - DNA, 30 bp, Artificial Sequence
  Description of Artificial Sequence Synthetic oligonucleotide
gtaattgcggcaagaagaattgtttctgtc

SEQ ID NO: 2 - DNA, 30 bp, Artificial Sequence
  Description of Artificial Sequence Synthetic oligonucleotide
gtaattgcggcaggaggaattgtttctgtc

SEQ ID NO: 3 - DNA, 30 bp, Artificial Sequence
  Description of Artificial Sequence Synthetic oligonucleotide
ggcaagagacaccaaagacactaccagcat

SEQ ID NO: 4 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 5 - protein, 11 aa, Homo sapiens
FEVAFLLIQWI

SEQ ID NO: 6 - protein, 11 aa, Homo sapiens
LLIQWYIGFSL

SEQ ID NO: 7 - protein, 16 aa, Homo sapiens
SLSAVYTCKRDPCPHQ

SEQ ID NO: 8 - protein, 12 aa, Homo sapiens
VDCFLSRPTEKT

SEQ ID NO: 9 - protein, 11 aa, Homo sapiens
SRPTEKTIFII

SEQ ID NO: 10 - protein, 7 aa, Homo sapiens
SRPTEKT

SEQ ID NO: 11 - protein, 13 aa, Homo sapiens
LGTAVESAWGDEQ

SEQ ID NO: 12 - protein, 12 aa, Homo sapiens
QSAFRCNTQQPG

SEQ ID NO: 13 - protein, 12 aa, Homo sapiens
QQPGCENVCYDK

SEQ ID NO: 14 - protein, 13 aa, Homo sapiens
VCYDKSFPISHVR

SEQ ID NO: 15 - protein, 18 aa, Homo sapiens
KRDPCHQVDCFLSRPTEK

SEQ ID NO: 16 - protein, 35 aa, Homo sapiens
ESAWGDEQSAFRCNTQQPGCENVCYDKSFPISHVR

SEQ ID NO: 17 - protein, 38 aa, Homo sapiens
LLIQWYIYGFSLSAVYTCKRDPCPHQVDCFLSRPTEKT

SEQ ID NO: 18 - protein, 42 aa, Homo sapiens
LLIQWYIYGFSLSAVYTCKRDPCPHQVDCFLSRPTEKTIFII

SEQ ID NO: 19 - protein, 11 aa, Artificial Sequence
  Description of Artificial Sequence Synthetic peptide
SRGGEKNVFIV

SEQ ID NO: 20 - DNA, 3130 bp, Homo sapiens
gagtcagtggcttgaaacttttaaaagctctgtgctccaagttacaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacata

SEQ ID NO: 21 - DNA, 27 bp, Artificial Sequence
  Description of Artificial Sequence Synthetic oligonucleotide
tcctgagcaatacctaacgaacaaata

SEQ ID NO: 22 - DNA, 21 bp, Artificial Sequence
  Description of Artificial Sequence Synthetic oligonucleotide
ctcagatagtggccagaatgc

SEQ ID NO: 23 - DNA, 20 bp, Artificial Sequence
  Description of Artificial Sequence Synthetic oligonucleotide
ttgtccaggtgactccaagg

SEQ ID NO: 24 - protein, 35 aa, Homo sapiens
KEVWGDEQADFVCNTLQPGCKNVCYDHYFPISHIR

SEQ ID NO: 25 - protein, 35 aa, Homo sapiens
QEVWGDEQEDFVCNTLQPGCKNVCYDHFFPVSHIR

SEQ ID NO: 26 - protein, 39 aa, Homo sapiens
MYVFYVMYDGFSMQRLVKCNAWPCPNTVDCFVSRPTEKT

SEQ ID NO: 27 - protein, 39 aa, Homo sapiens
MYVFYFLYNGYHLPWVLKCGIDPCPNLVDCFISRPTEKT

SEQ ID NO: 28 - protein, 43 aa, Homo sapiens
MYVFYVMYDGFSMQRLVKCNAWPCPNTVDCFVSRPTEKTVFTV

SEQ ID NO: 29 - protein, 42 aa, Homo sapiens
YVFYFLYNGYHLPWVLKCGIDPCPNLVDCFISRPTEKTVFTI

SEQ ID NO: 30 - DNA, 681 bp, Homo sapiens
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaa

SEQ ID NO: 31 - DNA, 786 bp, Homo sapiens
atggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagctaa